SEQUENCE LISTING (8 sequences)

SEQ ID NO: 1 - DNA, 52 bp, Homo sapiens
  misc_feature (27)..(27): n= A or C or G or T
ccacaagtctttgtggagagacgcacntggagaaaaactaacactcaacacc

SEQ ID NO: 2 - DNA, 51 bp, Homo sapiens
  misc_feature (27)..(27): n = A or G
tgttttcctcagacaggaccttgtcancgctttcaaatatgtaggctgttt

SEQ ID NO: 3 - DNA, 52 bp, Homo sapiens
  misc_feature (27)..(27): n = C or T
agacaggagggtcagaggtaagaatgntaccgctgggacagagaggaaggta

SEQ ID NO: 4 - DNA, 52 bp, Homo sapiens
  misc_feature (27)..(27): n = A or G
tcaagctttttgtagaagagtaaatgntgttgtgcttgtaacccaggaagct

SEQ ID NO: 5 - DNA, 52 bp, Homo sapiens
  misc_feature (27)..(27): n = A or G
agtactactagggctgagggaaggctnatatcctattgttagacagtgccag

SEQ ID NO: 6 - DNA, 52 bp, Homo sapiens
  misc_feature (27)..(27): n = C or T
attcttagtaatatttctttgcattcnaaggtttatcgctctaatcccacac

SEQ ID NO: 7 - DNA, 52 bp, Homo sapiens
  misc_feature (27)..(27): n = A or C
acccacaacaattagttccctgagatnttttcacaaagtccaggacccagta

SEQ ID NO: 8 - DNA, 52 bp, Homo sapiens
  misc_feature (27)..(27): n = G or T
cggcaaggctcagacctcggcatgtcngttacaagaagaattggagaagctc

(19) United States
(12) Patent Application Publication
(10) Pub. No.: US 2015/0044239 A1
(43) Pub. Date: Feb. 12, 2015

(54) Compositions and Methods for Diagnosing, Preventing and Treating Intracranial Aneurysms

(71) Applicants: Richard Lifton, New Haven, CT (US); Murat Gunel, Branford, CT (US)

(72) Inventors: Richard Lifton, New Haven, CT (US); Murat Gunel, Branford, CT (US)

(73) Assignee: YALE UNIVERSITY, New Haven, CT (US)

(21) Appl. No.: 14/009,063

(22) PCT Filed: Mar. 30, 2012
(86) PCT No.: PCT/US12/31518

§ 371 Date: Jan. 13, 2014

Related U.S. Application Data
(60) Provisional application No. 61/516,236, filed on Mar. 31, 2011.
(60) Provisional application No. 61/561,461, filed on Nov. 18, 2011.

(51) Int. Cl. C12Q 1/68; C12N 15/113; A61K 31/407; A61K 31/422; A61K 31/505; A61K 31/4025; A61K 38/02; C07K 16/18

(52) U.S. Cl. CPC C12Q 1/6883; A61K 38/02; C12N 15/113; C07K 16/18; A61K 31/422; A61K 31/505; A61K 31/4025; A61K 31/407; C12Q 2600/156; C12Q 2600/106; C12Q 2600/136; C12N 2310/141; C12N 2310/14; C12N 2310/12
USPC 424/184.1; 435/6.11; 514/1.1; 530/350; 514/44 A; 514/44 R; 530/387.1; 536/24.5; 514/380; 514/274; 514/422; 514/413

(57) ABSTRACT

The present invention relates to compositions and methods for diagnosing, preventing and treating intracranial aneurysm.

### STATEMENT REGARDING FEDERALLY SPONSORED RESEARCH OR DEVELOPMENT

This invention was made with government support from the U.S. National Institutes of Health (grant number R01NS057756). The U.S. government has certain rights in the invention.

### BACKGROUND OF THE INVENTION

Stroke is the third leading cause of death worldwide. Ten percent of strokes are due to intracranial hemorrhage, and the vast majority of these result from rupture of intracranial aneurysms (IAs). Intracranial aneurysms are balloon-like dilations of cerebral arteries and affect 2-5% of the population (Rinkel et al., 1998, Stroke, 29:251-256). Although most of these lesions are clinically silent, their rupture and consequent subarachnoid hemorrhage usually occurs between ages 40 and 60 without prior warning, resulting in substantial morbidity and mortality (International Study of Unruptured Intracranial Aneurysms Investigators, 1998, N Engl J Med, 339:1725-1733; Bederson et al., 2000, Stroke, 31:2742-2750). Intracranial aneurysm rupture results in 500,000 hemorrhagic strokes worldwide annually with devastating consequences: 45% of patients die within a month and 50% of the survivors are left with severe neurological deficits requiring long term care.

Given the very poor prognosis after cerebral hemorrhage, diagnosis of aneurysm prior to rupture is paramount, as surgical or endovascular repair can prevent morbidity and mortality. Aside from the well-established risk factors, such as hypertension, smoking, female sex (Nahed et al., 2007, Neurosurgery, 60:213-226), and high shear stress imposed on the cerebrovasculature (Nixon et al., 2010, J Neurosurg, 112:1240-125), there is evidence for significant genetic contribution to intracranial aneurysm pathogenesis (Bilguvar et al., 2008, Nat Genet, 40:1472-1477). Siblings of intracranial aneurysm probands are at an estimated 4-fold increased risk of hemorrhagic stroke due to intracranial aneurysm compared to the general population, suggesting a significant genetic component to risk; the pattern of recurrence is generally consistent with multifactorial determination.

While hypertension, smoking and positive family history increase the likelihood of intracranial aneurysm formation and/or rupture, these are of limited clinical value in identifying at-risk individuals. Thus, there is need in the art for screening measures to identify subjects at risk for developing IA, as well as for methods of preventing and treating IA. The present invention fulfills these needs.

### SUMMARY OF THE INVENTION

The present invention relates to the discovery that particular single nucleotide polymorphisms (SNPs) are associated with intracranial aneurysm. The invention relates to compositions and methods useful for the diagnosis, assessment, characterization and treatment of intracranial aneurysm, based upon the presence or absence of an SNP that is associated with intracranial aneurysm. The methods of the invention relate to methods of assessing a subject's risk of having or developing an intracranial aneurysm, methods of identifying modulators of intracranial aneurysm, methods of preventing intracranial aneurysm and methods of treating intracranial aneurysm.

In one embodiment, the invention is method of identifying an single nucleotide polymorphism (SNP) associated with intracranial aneurysm in a biological sample of a subject in need thereof, including the steps of: obtaining a biological sample from the subject, determining the sequence of at least a portion of the subject's nucleic acid, comparing the subject's nucleic acid sequence to a wild-type nucleic acid sequence, and identifying at least one SNP in the subject's nucleic acid sequence as compared with the wild-type nucleic acid sequence, where the identified SNP is associated with intracranial aneurysm. In various embodiments, the nucleic acid is genomic DNA, mRNA, cDNA or a combination thereof. In various embodiments, the SNP is rs6841581, rs9298506, rs1333040, rs12413409, rs6538595, rs9315204, rs11661542, 1132274, or a combination thereof. In one embodiment, the subject is human. In some embodiments, the method employs PCR. In various embodiments, the biological sample is blood, plasma, serum, a body fluid, a cell, a tissue or a combination thereof.

In another embodiment, the invention is a method of diagnosing a subject as having, or as being at risk of developing, intracranial aneurysm, including the steps of: obtaining a biological sample from the subject, determining the sequence of at least a portion of the subject's nucleic acid, comparing the subject's nucleic acid sequence to a wild-type nucleic acid sequence, and identifying at least one SNP in the subject's nucleic acid sequence as compared with the wild-type nucleic acid sequence, where the identified SNP is associated with intracranial aneurysm. In various embodiments, the nucleic acid is genomic DNA, mRNA, cDNA or a combination thereof. In various embodiments, the SNP is rs6841581, rs9298506, rs1333040, rs12413409, rs6538595, rs9315204, rs11661542, 1132274, or a combination thereof. In one embodiment, the subject is human. In some embodiments, the method employs PCR. In various embodiments, the biological sample is blood, plasma, serum, a body fluid, a cell, a tissue or a combination thereof.

In a further embodiment, the invention is a method of treating intracranial aneurysm in a subject in need thereof, including the steps of: administering to the subject, a therapeutically effective amount of a modulator of a gene, or gene product, that is associated with an SNP that is associated with intracranial aneurysm, where the subject has been diagnosed as having an intracranial aneurysm, and where after the modulator is administered to the subject, the intracranial aneurysm is treated. In various embodiments, the modulator is a chemical compound, a polypeptide, a peptide, a peptidomemetic, an antibody, a ribozyme, a small molecule chemical compound, an antisense nucleic acid molecule, or a combination thereof. In one embodiment, the subject has at least one SNP associated with intracranial aneurysm. In various embodiments, the SNP associated with intracranial aneurysm is rs6841581, rs9298506, rs1333040, rs12413409, rs6538595, rs9315204, rs11661542, rs1132274 or a combination thereof. In some embodiments, the gene, or gene product, that is associated with an SNP associated with intracranial aneurysm is EDNRA, SOX17, CDKN2A, CDKN2B, CNNM2, NDUFA12, NR2C1, FGD6, VEZT, KL, STARD13, RBBP8, DSTN, RRBP1 or a combination thereof. In one embodiment, the subject is human.

In one embodiment, the invention is a method of preventing intracranial aneurysm in a subject in need thereof, including the steps of: administering to the subject, a therapeutically effective amount of a modulator of a gene, or gene product, that is associated with an SNP that is associated with intracranial aneurysm, where the subject has been diagnosed as having an intracranial aneurysm, and where after the modulator is administered to the subject, the intracranial aneurysm is treated. In various embodiments, the modulator is a chemical compound, a polypeptide, a peptide, a peptidomemetic, an antibody, a ribozyme, a small molecule chemical compound, an antisense nucleic acid molecule, or a combination thereof. In one embodiment, the subject has at least one SNP associated with intracranial aneurysm. In various embodiments, the SNP associated with intracranial aneurysm is rs6841581, rs9298506, rs1333040, rs12413409, rs6538595, rs9315204, rs11661542, rs1132274 or a combination thereof. In some embodiments, the gene, or gene product, that is associated with an SNP associated with intracranial aneurysm is EDNRA, SOX 17, CDKN2A, CDKN2B, CNNM2, NDUFA12, NR2C1, FGD6, VEZT, KL, STARD13, RBBP8, DSTN, RRBP1 or a combination thereof. In one embodiment, the subject is human.

In another embodiment, the invention is a method of identifying a test compound as a modulator of a gene, or gene product, that is associated with an SNP associated with intracranial aneurysm, including the steps of: measuring at least one parameter of a gene, or gene product, that is associated with an SNP associated with intracranial aneurysm in the absence of the test compound, measuring the at least one parameter of a gene, or gene product, that is associated with an SNP associated with intracranial aneurysm in the presence of the test compound; comparing the measurement of the at least one parameter of a gene, or gene product, that is associated with an SNP associated with intracranial aneurysm in the presence of the test compound with the measurement in the absence of the test compound; identifying the test compound as a modulator of the gene, or gene product, that is associated with an SNP associated with intracranial aneurysm when the measurement of the parameter in the presence of the test compound is different than the measurement of the parameter in the absence of the test compound. When the measurement of the parameter is higher in the presence of the test compound, the test compound is identified as an activator. When the measurement of the parameter is lower in the presence of the test compound, the test compound is identified as an inhibitor. In various embodiments, the test compound is a chemical compound, a polypeptide, a peptide, a peptidomemetic, an antibody, a nucleic acid, an antisense nucleic acid, an shRNA, a ribozyme, a small molecule chemical compound, or a combination thereof. In another embodiment, the invention is a modulator identified using the method of identifying a test compound as a modulator. In some embodiments, the modulator is an activator. In other embodiments, the modulator is an inhibitor. In a further embodiment, the invention is a composition comprising the modulator identified using the method of identifying a test compound as a modulator

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration depicting the distribution of the PPA along the genome. The PPAs for SNPs analyzed previously (Yasuno et al., 2010, Nat Genet, 42:420-425) are plotted along the genomic coordinates [National Center for Biotechnology Information (NCBI) build 36]. 767,877 SNPs with PPA<0.0001 were omitted from a total of 831,532 SNPs. Fifteen analysed regions (i.e., those regions containing a SNP with 0.1<PPA<0.5 but no SNPs with PPA>0.5) are shaded in gray.

FIG. 2 depicts regional plots for associated regions. For each chromosomal interval, −log₁₀ P values for association test are plotted against the genomic coordinates (NCBI build 36) (Upper); the recombination rates obtained from the HapMap database and the RefSeq genes (hg18) within the regions (Lower). The rs identifier of the SNP listed in Table 2 is shown for each chromosomal interval, and its position is indicated by the vertical line (Upper). Dark and light dots represent results of the genotyped and imputed SNPs for the discovery cohort, respectively. Squares represent the association result from the replication cohort using the JP1 plus JP2 (rs6541581 and rs6538595) or JP2-only (rs1132274); combined P values of the discovery and replication cohorts based on the fixed-effects model are shown by diamonds.

FIG. 3 depicts the results of example experiments which demonstrate the consistency of associations across cohorts. Forest plots are shown for meta-analysis of SNPs listed in Table 2. Squares and horizontal segments represent estimated per-allele ORs and 95% confidence intervals (CIs) for individual cohorts. Diamonds represent the summary OR estimates and 95% CIs for the meta-analyses of six cohorts (fixed- and random-effects models). A log₁₀ (BF)>0 supports association with IA, and a log₁₀(BF)<0 supports no association with IA.

FIG. 4, comprising FIG. 4A through FIG. 4C, depicts the results of the genome-wide association analysis in the discovery cohort. FIG. 4A depicts the PPAs for 831,532 quality control-passed SNPs that were analyzed specifying a prior probability of association of 1/10,000, plotted against genomic locations of SNPs. A gray horizontal line at PPA=0.5 indicates the cutoff value for follow-up genotyping. FIG. 4B depicts quantile-quantile plots of P values (−log₁₀ scale) for all the SNPs analyzed (black; n=831,532); for SNPs after excluding those within previously identified regions (red; n=830,907); and for SNPs after excluding all within the final associated intervals (blue; n=830,158). FIG. 4C depicts a scatter plot of −log₁₀ P versus log₁₀ Bayes factors with color for each point indicating the range of PPA values. There are very close relationships among the P values for association, the Bayes factor and the PPA value. Note that, given a uniform prior probability of association, the PPA increases as the Bayes factor increases. A vertical line indicates the minimum PPA threshold at 0.5 (Bayes factor=1.0×10⁴) for follow-up.

FIG. 5, depicts regional plots for associated regions. For each chromosomal interval, −log₁₀ P values for association are plotted against the genomic coordinates (NCBI build 36) in the panel above; the recombination rates obtained from the HapMap database and the RefSeq genes (hg18) within the regions are shown in the panel below. Above, rs identifiers of SNPs listed in Table 9 are shown and their positions are indicated by gray vertical lines. Gray dashed lines indicate locations of other SNPs genotyped in the replication cohorts. Dark blue and light blue dots represent results of genotyped and imputed SNPs for the discovery cohort, respectively; orange and light orange squares represent association results for the replication cohort using JP1 combined with JP2 and also JP2-only, respectively; combined results for SNPs genotyped both in the discovery and the replication cohort using JP1 plus JP2 and JP2-only are shown by red and light red diamonds, respectively.

FIG. 6 depicts the results of experiments which demonstrate the consistency of association across cohorts. Forest plots are shown for meta-analysis of the SNPs listed in Table 9. Squares and horizontal segments represent estimated per-allele ORs and 95% CIs for individual cohorts. Diamonds represent the summary OR estimates and 95% CIs for the meta-analyses of six cohorts (using fixed- and random-effects models). Log₁₀ (Bayes factor)>0 supports association with intracranial aneurysm (IA), whereas log₁₀ (Bayes factor)<0 supports no association with intracranial aneurysm. Analyzing the results here as six distinct cohorts rather than four cohorts (as in the primary analysis) resulted in only minor differences due to different weights given to sub-cohorts of the combined European cohort.

FIG. 7 depicts the results of conditional analysis in the discovery cohort. Conditional analysis for each of the associated regions was performed using only the genotyped SNPs in the discovery cohort. Location of each diamond represents the P-value after adjusting for the effect of the conditioned SNP, while the other end of the line connected to a particular diamond shows the P-value of a single-locus analysis, prior to conditioning. Test statistics were not corrected using genomic control. SNPs listed in Table 9 were used as the conditioned ones, which are indicated by yellow diamonds. The genotypic squared correlation between the conditioned SNP and a tested SNP is color-coded as indicated in the inset within the top left panel. An interval [x,y) for r2 indicates x≦r²<y. Very strongly correlated SNPs with r²≧0.8 were not analyze because of multiple colinearity. The intervals shown are the same as in FIG. 5.

FIG. 8 depicts the Receiver-Operating Characteristic curve from 5 associated loci. Prediction using 5 SNPs that are associated with intracranial aneurysm (Table 9) is shown in blue for 3 ethnically distinct cohorts. These curves were obtained by fitting conditional (Finnish and other combined European) or unconditional logistic regression models as described elsewhere herein and by calculating the proportion of cases and controls with risk scores exceeding each possible value. The black curves correspond to the theoretical curves for the polygenic model with the sibling recurrence risk (λₛ)=1.075 (Finnish), 1.058 (other combined European) and 1.051, respectively (Clayton, 2009, PLoS Genet, 5: e1000540). The dished curves correspond to the epidemiologically estimated value, a λ_{S}=4 (Cannon Albright et al., 2003, J Neurosurg, 99, 637-643; Schievink, 1997, Neurosurgery, 40: 651-663).

### DETAILED DESCRIPTION OF THE INVENTION

The present invention relates to the discovery that particular single nucleotide polymorphisms (SNPs) are associated with intracranial aneurysm. The invention relates to compositions and methods useful for the diagnosis, assessment, characterization and treatment of intracranial aneurysm, based upon the presence or absence of an SNP that is associated with intracranial aneurysm.

The methods of the invention relate to methods of assessing a subject's risk of having or developing an intracranial aneurysm, methods of identifying modulators of intracranial aneurysm, methods of preventing intracranial aneurysm and methods of treating intracranial aneurysm.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “allele” refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variations,” “variants,” “polymorphisms,” or “mutations.”

As used herein, to “alleviate” a disease or disorder means reducing the frequency or severity of at least one sign or symptom of a disease or disorder, such as intracranial aneurysm.

As used herein the terms “alteration,” “defect,” “variation,” “mutation,” or “polymorphism” refers to a variation in a nucleic acid sequence in a cell that affects the function, activity, expression (transcription or translation) or conformation of the polypeptide that it encodes. Variations encompassed by the present invention can be any variation of a nucleic acid in a cell that results in the enhancement or disruption of the function, activity, expression or conformation of the encoded RNA or encoded polypeptide, including the complete absence of expression of the encoded RNA or encoded protein and can include, for example, missense and nonsense mutations, insertions, deletions, frameshifts and premature terminations. Without being so limited, variations encompassed by the present invention may alter the regulation of the expression of the encoded RNA, the expression level of the encoded RNA, the splicing the mRNA (splice site mutation) or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci, USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “applicator,” as the term is used herein, is meant any device including, but not limited to, a hypodermic syringe, a pipette, an iontophoresis device, a patch, and the like, for administering the compositions of the invention to a subject.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the term “control nucleic acid” is meant to refer to a nucleic acid (e.g., RNA, DNA) that does not come from a subject known to have, or suspected to have, a mutation or polymorphism in the nucleic acid of interest (e.g., for a control subject). For example, the control can be a wild type nucleic acid sequence which does not contain a variation in its nucleic acid sequence. Also, as used herein, a control nucleic acid can be a fragment or portion of gene that does not include the variation that is the mutation or polymorphism of interest (that is, the variation to be detected in an assay).

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “diagnosis” refers to the determination of the nature of a case of disease or disorder. In some embodiments of the present invention, methods for making a diagnosis are provided which permit determination of a particular SNP associated with intracranial aneurysm.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

The term “gene” refers to a nucleic acid (e.g., DNA) sequence that includes coding sequences necessary for the production of a polypeptide, precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “genome” is all the genetic material of an organism. In some instances, the term genome may refer to the chromosomal DNA. Genome may be multichromosomal such that the DNA is cellularly distributed among a plurality of individual chromosomes. For example, in the human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “housekeeping gene” as used herein refers to genes that are generally always expressed and thought to be involved in routine cellular metabolism. Housekeeping genes are well known and include such genes as glyceraldehyde-3-phosphate dehydrogenase (G3PDH or GAPDH), albumin, actins, tubulins, cyclophilin, hypoxanthine phsophoribosyltransferase (HRPT), 28S, and 18S rRNAs and the like.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” A single DNA molecule with internal complementarity could assume a variety of secondary structures including loops, kinks or, for long stretches of base pairs, coils.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The terms “microarray” and “array” refers broadly to both “DNA microarrays” and “DNA chip(s),” and encompasses all art-recognized solid supports, and all art-recognized methods for affixing nucleic acid molecules thereto or for synthesis of nucleic acids thereon. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, SNP analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, Arrays and methods of genetic analysis using arrays is described in Cutler, et al., 2001, Genome Res. 11(11): 1913-1925 and Warrington, et al., 2002, Hum Mutat 19:402-409 and in U.S. Patent Pub No 20030124539, each of which is incorporated herein by reference in its entirety.

By the term “modulating,” as used herein, is meant mediating a detectable increase or decrease in the level or activity of a molecule, or in the response in a subject, compared with the level or activity of a molecule, or in the response in the subject, in the absence of a treatment or compound, and/or compared with the level or activity of an otherwise identical but untreated molecule or of a response in an otherwise identical but untreated subject. The term encompasses perturbing and/or affecting a native signal or response thereby mediating a beneficial therapeutic response in a subject, preferably, a human.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences.

The term “perfect match,” “match,” “perfect match probe” or “perfect match control” refers to a nucleic acid that has a sequence that is perfectly complementary to a particular target sequence. The nucleic acid is typically perfectly complementary to a portion (subsequence) of the target sequence. A perfect match (PM) probe can be a “test probe”, a “normalization control” probe, an expression level control probe and the like. A perfect match control or perfect match is, however, distinguished from a “mismatch” or “mismatch probe.” The term “mismatch,” “mismatch control” or “mismatch probe” refers to a nucleic acid whose sequence is not perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “reaction mixture” or “PCR reaction mixture” or “master mix” or “master mixture” refers to an aqueous solution of constituents in a PCR reaction that can be constant across different reactions. An exemplary PCR reaction mixture includes buffer, a mixture of deoxyribonucleoside triphosphates, primers, probes, and DNA polymerase. Generally, template RNA or DNA is the variable in a PCR.

“Sample” or “biological sample” as used herein means a biological material isolated from a subject. The biological sample may contain any biological material suitable for detecting an SNP, gene, or gene product associated with intracranial aneurysm, and may comprise fluid, cellular and/or non-cellular material obtained from the individual.

A “single-nucleotide polymorphism” (SNP) refers to a nucleic sequence variation of a single nucleotide in a nucleic acid sequence that differs between subjects of the same species or between paired chromosomes in a subject. SNPs may fall within coding sequences of genes, within the non-coding regions of genes, or within the intergenic regions between genes. SNPs that are not in protein-coding regions may still affect gene expression levels through, by way of examples, gene splicing, transcription factor binding, or messenger RNA degradation.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, “substantially purified” refers to being essentially free of other components. For example, a substantially purified cell is a cell which has been separated from other cell types with which it is normally associated in its naturally occurring state. In some instances, a population of substantially purified cells refers to a homogenous population of cells. In other instances, this term refers simply to a cell that have been separated from the cells with which they are naturally associated in their natural state.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Targets are sometimes referred to in the art as anti-probes. As the term target is used herein, no difference in meaning is intended.

As used herein, the terms “therapy” or “therapeutic regimen” refer to those medical steps taken to alleviate or alter a disorder or disease state, e.g., a course of treatment intended to reduce or eliminate the signs or symptoms of a disease or disorder using pharmacological, surgical, dietary and/or other techniques. A therapeutic regimen may include a prescribed dosage of one or more drugs or surgery. Therapies will most often be beneficial and reduce the disorder or disease state but in many instances the effect of a therapy will have non-desirable or side-effects. The effect of therapy will also be impacted by the physiological state of the host, e.g., age, gender, genetics, weight, other disease conditions, etc.

The term “therapeutically effective amount” refers to the amount of the subject modulator compound that will elicit the biological or medical response of a tissue, system, or subject that is being sought by the researcher, veterinarian, medical doctor or other clinician. The term “therapeutically effective amount” includes that amount of a modulator compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

As used herein, the term “wild-type” refers to a nucleic acid or nucleic product isolated from a naturally occurring source. A wild-type nucleic acid is that which is most frequently observed in a population and is thus arbitrarily designed the “normal” or “wild-type” form of the nucleic acid. In contrast, the term “modified” or “mutant” or “polymorphic” refers to a nucleic acid or nucleic acid product that displays modifications in sequence and/or functional properties (i.e., altered characteristics) when compared to the wild-type nucleic acid or nucleic acid product. It is noted that naturally occurring variations can be isolated; these are identified by the fact that they have altered characteristics (including altered nucleic acid sequences) when compared to the wild-type nucleic acid or nucleic acid product.

### DESCRIPTION

The present invention relates to the discovery that particular single nucleotide polymorphisms (SNPs) are associated with intracranial aneurysm. The invention relates to compositions and methods useful for the diagnosis, assessment, characterization and treatment of intracranial aneurysm, based upon the presence or absence of an SNP that is associated with intracranial aneurysm.

The methods of the invention relate to methods of assessing a subject's risk of having or developing an intracranial aneurysm, methods of identifying modulators of intracranial aneurysm, methods of preventing intracranial aneurysm and methods of treating intracranial aneurysm.

In various embodiments, the compositions and methods of the invention relate to an SNP that is associated with intracranial aneurysm, genes associated with an SNP associated with intracranial aneurysm, gene products associated with an SNP associated with intracranial aneurysm, and modulators of genes and gene products associated with an SNP associated with intracranial aneurysm.

In other various embodiments, the methods of the invention relate to methods of diagnosing intracranial aneurysm, methods of characterizing intracranial aneurysm, methods of identifying modulators of intracranial aneurysm, methods of preventing intracranial aneurysm, and methods of treating intracranial aneurysm.

In various embodiments of the compositions and methods of the invention described herein, the SNP associated with intracranial aneurysm is at least one of rs6841581, rs9298506, rs1333040, rs12413409, rs6538595, rs9315204, rs11661542, or rs1132274. In other various embodiments, the gene associated with an SNP associated with intracranial aneurysm is at least one of EDNRA, SOX17, CDKN2A, CDKN2B, CNNM2, NDUFA12, NR2C1, FGD6, VEZT, KL, STARD13, RBBP8, DSTN, or RRBP1.

### Assays

The present invention relates to the discovery that particular SNPs are associated with the development and progression of intracranial aneurysm. In various embodiments, the invention relates to a genetic screening assay of a subject to determine whether the subject has SNP associated with intracranial aneurysm. The present invention provides methods of assessing for the presence or absence of a SNP associated with intracranial aneurysm, as well as methods of diagnosing a subject as having, or as being at risk of developing, intracranial aneurysm based upon the presence of the SNP associated with intracranial aneurysm. In some embodiments, the diagnostic assays described herein are in vitro assays. In other embodiments, the diagnostic assays described herein are in vivo assays.

In one embodiment, the method of the invention is a diagnostic assay for diagnosing intracranial aneurysm in a subject in need thereof, by determining whether an SNP associated with intracranial aneurysm is present in a biological sample obtained from the subject. The results of the diagnostic assay can be used alone, or in combination with other information from the subject, or from the biological sample obtained from the subject. In some embodiments, the diagnostic assay of the invention is an in vitro assay. In other embodiments, the diagnostic assay of the invention is an in vivo assay. The SNP identified by the assay can be any SNP that is associated with an intracranial aneurysm. In some embodiments, the SNP is at least one of rs6841581, rs9298506, rs1333040, rs12413409, rs6538595, rs9315204, rs11661542, or rs1132274.

In the assay methods of the invention, a test biological sample from a subject is assessed for the presence of at least SNP associated with intracranial aneurysm. The test biological sample can be an in vitro sample or an in vivo sample. In various embodiments, the subject is a human subject, and may be of any race, sex and age. Representative subjects include those who are suspected of having intracranial aneurysm, those who have been diagnosed with intracranial aneurysm, those whose have intracranial aneurysm, those who have had an intracranial aneurysm, those who at risk of a recurrence of intracranial aneurysm, and those who are at risk of developing intracranial aneurysm.

In some embodiments, an intracranial aneurysm associated SNP-binding molecule is used in vivo for the diagnosis of intracranial aneurysm. In some embodiments, the intracranial aneurysm associated SNP-binding molecule is nucleic acid that hybridizes with an intracranial aneurysm associated SNP.

In one embodiment, the test sample is a sample containing at least a fragment of a nucleic acid comprising an SNP associated with intracranial aneurysm. The term, “fragment,” as used herein, indicates that the portion of a nucleic acid (e.g., DNA, mRNA or cDNA) that is sufficient to identify it as comprising an SNP associated with intracranial aneurysm. In representative embodiments, a fragment comprises one or more exons, one or more introns, and/or one or more intragenic regions. In other representative embodiments, a fragment comprises part of at least one exon, part of at least one intron, and/or part of at least one intragenic region.

In some embodiments, the test sample is prepared from a biological sample obtained from the subject. The biological sample can be a sample from any source which contains a nucleic acid comprising an intracranial aneurysm associated SNP (e.g., DNA, chromosomal nucleic acid, or RNA), such as a body fluid (e.g., blood, plasma, serum, etc.), or a tissue, or a cell, or a combination thereof. A biological sample can be obtained by appropriate methods, such as, by way of examples, biopsy or fluid draw. In certain embodiments, a biological sample containing genomic DNA is used. The biological sample can be used as the test sample; alternatively, the biological sample can be processed to enhance access to polypeptides, nucleic acids, or copies of nucleic acids (e.g., copies of nucleic acids comprising an SNP associated with intracranial aneurysm), and the processed biological sample can then be used as the test sample. For example, in various embodiments, nucleic acid (e.g., genomic DNA or cDNA prepared from RNA) is prepared from a biological sample, for use in the methods. Alternatively or in addition, if desired, an amplification method can be used to amplify nucleic acids comprising all or a fragment of an RNA or genomic DNA in a biological sample, for use as the test sample in the assessment for the presence or absence of an SNP associated with intracranial aneurysm.

The test sample is assessed to determine whether one or more SNPs associated with intracranial aneurysm are present in the nucleic acid of the subject. In general, detecting an SNP may be carried out by determining the presence or absence of a nucleic acid containing an SNP of interest in the test sample.

In some embodiments, hybridization methods, such as Southern analysis, Northern analysis, or in situ hybridizations, can be used (see Current Protocols in Molecular Biology, Ausubel, F. et al., eds., John Wiley & Sons, including all supplements). For example, the presence of an SNP associated with intracranial aneurysm can be indicated by hybridization of nucleic acid in the genomic DNA, RNA, or cDNA to a nucleic acid probe. A “nucleic acid probe”, as used herein, can be a DNA probe or an RNA probe; the nucleic acid probe can contain at least one polymorphism of interest, as described herein. The probe can be, for example, the gene, a gene fragment (e.g., one or more exons), a vector comprising the gene, a probe or primer, etc. For representative examples of use of nucleic acid probes, see, for example, U.S. Pat. Nos. 5,288,611 and 4,851,330.

To detect one or more SNPs of interest, a hybridization sample is formed by contacting the test sample with at least one nucleic acid probe. A preferred probe for detecting RNA, cDNA or genomic DNA is a labeled nucleic acid probe capable of hybridizing to RNA, cDNA or genomic DNA. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target RNA, cDNA or genomic DNA. The hybridization sample is maintained under conditions which are sufficient to allow specific hybridization of the nucleic acid probe to RNA, cDNA or genomic DNA. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a polymorphic sequence and a gene, RNA or cDNA in the test sample, the polymorphism that is present in the nucleic acid probe is also present in the nucleic acid sequence of the subject. More than one nucleic acid probe can also be used concurrently in this method. Specific hybridization of any one of the nucleic acid probes is indicative of the presence of the SNP of interest, as described herein.

In Northern analysis (see Current Protocols in Molecular Biology, Ausubel, F. et al., eds., John Wiley & Sons, supra), the hybridization methods described above are used to identify the presence of an SNP of interest in an RNA, such as unprocessed, partially processed or fully processed mRNA. For Northern analysis, a test sample comprising RNA is prepared from a biological sample from the subject by appropriate means. Specific hybridization of a nucleic acid probe, as described above, to RNA from the subject is indicative of the presence of an SNP of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising one or more SNPs of interest. Hybridization of the PNA probe to a nucleic acid sequence is indicative of the presence of an SNP of interest.

In another embodiment of the methods of the invention, analysis by restriction digestion can be used to detect an SNP of interest in a nucleic acid, if the SNP results in the creation or elimination of a restriction site. A sample containing nucleic acid from the subject is used. Polymerase chain reaction (PCR) can be used to amplify all or a fragment of a nucleic acid (including, when necessary, the flanking sequences) in the sample. RFLP analysis is conducted as described (see Current Protocols in Molecular Biology, supra). The digestion pattern of the relevant fragments indicates the presence or absence of an SNP of interest.

Direct sequence analysis can also be used to detect SNPs of interest. A sample comprising DNA or RNA can be used, and PCR or other appropriate methods can be used to amplify all or a fragment of the nucleic acid, and/or its flanking sequences, if desired. The nucleic acid, or a fragment thereof (e.g., one or more exons, one or more introns, one or more intragenic regions, etc.), or cDNA, or fragment of the cDNA, or RNA, or fragment of the RNA, is determined, using standard methods. The sequence of the genomic DNA, genomic DNA fragment, gene, gene fragment, cDNA, cDNA fragment, RNA, or RNA fragment is compared with a comparator nucleic acid sequence, such as a wild-type nucleic acid, as appropriate. The presence or absence of an SNP is then identified.

Allele-specific oligonucleotides can also be used to detect the presence of an SNP of interest through, for example, the use of dot-blot hybridization of amplified oligonucleotides with allele-specific oligonucleotide (ASO) probes (see, for example, 1986, Saiki et al., Nature 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to the SNP sequence. An allele-specific oligonucleotide probe that is specific for a particular SNP can be prepared, using standard methods (see Current Protocols in Molecular Biology, supra). To identify a an SNP of interest, a sample comprising nucleic acid is used. PCR can be used to amplify all or a fragment of the test nucleic acid sequence. The nucleic acid containing the amplified sequence (or fragment thereof) is dot-blotted, using standard methods (see Current Protocols in Molecular Biology, supra), and the blot is contacted with the oligonucleotide probe. The presence of specific hybridization of the probe to the amplified nucleic acid is then detected. Specific hybridization of an allele-specific oligonucleotide probe containing the SNP of interest, to test nucleic acid from the subject is indicative of the presence of the SNP of interest.

In another embodiment of the invention, fluorescence resonance energy transfer (FRET) can be used to detect the presence of a SNP. FRET is the process of a distance-dependent excited state interaction in which the emission of one fluorescent molecule is coupled to the excitation of another. A typical acceptor and donor pair for resonance energy transfer consists of 4-[[4-(dimethylamino) phenyl]azo]benzoic acid (DABCYL) and 5-[(2-aminoethylamino]naphthalene sulfonic acid (EDANS). EDANS is excited by illumination with 336 nm light, and emits a photon with wavelength 490 n×n. If a DABCYL moiety is located within 20 angstroms of the EDANS, this photon will be efficiently absorbed. DABCYL and MANS will be attached to two different oligonucleotide probes designed to hybridize head-to-tail to nucleic acid adjacent to and/or overlapping the site of one of the SNPs of interest. Melting curve analysis is then applied: cycles of denaturation, cooling, and re-heating are applied to a test sample mixed with the oligonucleotide probes, and the fluorescence is continuously monitored to detect a decrease in DABCYL fluorescence or an increase in EDANS fluorescence (loss of quenching). While the two probes remain hybridized adjacent to one another, FRET will be very efficient. Physical separation of the oligonucleotide probes results in inefficient FRET, as the two dyes are no longer in close proximity. The presence or absence of an SNP of interest can be assessed by comparing the fluorescence intensity profile obtained from the test sample, to fluorescence intensity profiles of control samples comprising known SNPs of interest.

In another embodiment, arrays of oligonucleotide probes that are complementary to target nucleic acid sequences from a subject can be used to detect and identify SNPs associated with intracranial aneurysm. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and scanned for SNPs. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target nucleic acid sequence which includes one or more previously identified SNPs or markers is amplified by well-known amplification techniques, e.g., PCR. Typically, this involves the use of primer sequences that are complementary to the two strands of the target sequence both upstream and downstream of the SNP. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although often described in terms of a single detection block (e.g., for detection of a single variations), arrays can include multiple detection blocks, and thus be capable of analyzing multiple, specific variations. In alternate arrangements, it will generally be understood that detection blocks may be grouped within a single array or in multiple, separate arrays so that varying, optimal conditions may be used during the hybridization of the target to the array. This allows for the separate optimization of hybridization conditions for each situation. Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect SNPs of interest. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (1981, Sheffield et al., Proc. Natl. Acad. Sci, USA 86:232-236), mobility shift analysis (1989, Orita et al., Proc. Natl. Acad. Sci. USA 86:2766-2770; 1987, Rosenbaum and Reissner, Biophys. Chem. 265:1275; 1991, Keen et al., Trends Genet. 7:5); restriction enzyme analysis (1978, Flavell et al., Cell 15:25; 1981, Geever, et al., Proc. Natl. Acad. Sci. USA 78:5081); heteroduplex analysis; chemical mismatch cleavage (CMC) (1985, Cotton et al., Proc, Natl. Acad. Sci. USA 85:4397-4401); RNase protection assays (1985, Myers, et al., Science 230:1242); use of polypeptides which recognize nucleotide mismatches, such as E. coli mutS protein (see, for example, U.S. Pat. No. 5,459,039); Luminex xMAP™ technology; high-throughput sequencing (HTS) (2011, Gundry and Vijg, Mutat Res, doi:10.1016/j.mrfmmm.2011.10.001); next-generation sequencing (NGS) (2009, Voelkerding et al., Clinical Chemistry 55:641-658; 2011, Su et al., Expert Rev Mol. Diagn. 11:333-343; 2011, Ji and Myllykangas, Biotechnol Genet Eng Rev 27:135-158); ion semiconductor sequencing (2011, Rusk, Nature Methods doi:10.1038/nmreth.f.330; 2011, Rothberg et al., Nature 475:348-352) and/or allele-specific PCR, for example. These and other methods can be used to identify the presence of one or more SNPs of interest, in a biological sample obtained from a subject. In one embodiment of the invention, the methods of assessing a biological sample for the presence or absence of an SNP, as described herein, are used to diagnose intracranial aneurysm in a subject in need thereof.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the cells using known techniques. Nucleic acid herein refers to RNA, including mRNA, and DNA, including genomic DNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a fresh or fixed biological sample.

Routine methods also can be used to extract genomic DNA from a tissue sample, including, for example, phenol extraction. Alternatively, genomic DNA can be extracted with kits such as the QIAamp™. Tissue Kit (Qiagen, Chatsworth, Calif.), the Wizard™ Genomic DNA purification kit (Promega, Madison, Wis.), the Puregene DNA Isolation System (Gentra Systems, Inc., Minneapolis, Minn.), and the A.S.A.P.™ Genomic DNA isolation kit (Boehringer Mannheim, Indianapolis, Ind.).

There are many methods known in the art for the detection of specific nucleic acid sequences and new methods are continually reported. A great majority of the known specific nucleic acid detection methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. No. 6,159,693 and No. 6,270,974, and related patents. To briefly summarize one of those methods, a nucleic acid probe of at least 10 nucleotides, preferably at least 15 nucleotides, more preferably at least 25 nucleotides, having a sequence complementary to a desired region of the target nucleic acid of interest is hybridized in a sample, subjected to depolymerizing conditions, and the sample is treated with an ATP/luciferase system, which will luminesce if the nucleic sequence is present. In quantitative Southern blotting, levels of the polymorphic nucleic acid can be compared to wild-type levels of the nucleic acid.

A further process for the detection of hybridized nucleic acid takes advantage of the polymerase chain reaction (PCR). The PCR process is well known in the art (U.S. Pat. No. 4,683,195, U.S. Pat. No. 4,683,202, and U.S. Pat. No. 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

DNA amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable DNA polymerase. The extension product is then denatured from the target sequence by heating, and the process is repeated. Successive cycling of this procedure on both DNA strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

The expression specifically hybridizing in stringent conditions refers to a hybridizing step in the process of the invention where the oligonucleotide sequences selected as probes or primers are of adequate length and sufficiently unambiguous so as to minimize the amount of non-specific binding that may occur during the amplification. The oligonuecleotide probes or primers herein described may be prepared by any suitable methods such as chemical synthesis methods.

Hybridization is typically accomplished by annealing the oligonucleotide probe or primer to the DNA under conditions of stringency that prevent non-specific binding but permit binding of this DNA which has a significant level of homology with the probe or primer.

Among the conditions of stringency is the melting temperature (Tin) for the amplification step using the set of primers, which is in the range of about 55° C. to about 70° C. Preferably, the Tm for the amplification step is in the range of about 59° C. to about 72° C. Most preferably, the Tin for the amplification step is about 60° C.

Typical hybridization and washing stringency conditions depend in part on the size (i.e., number of nucleotides in length) of the DNA or the oligonucleotide probe, the base composition and monovalent and divalent cation concentrations (Ausubel et al., 1994, eds Current Protocols in Molecular Biology).

In a preferred embodiment, the process for determining the quantitative and qualitative profile according to the present invention is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In another preferred embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a nucleic acid sequence flanking the SNP of interest, and more particularly the primer includes 12 or more contiguous nucleotides substantially complementary to the sequence flanking the SNP of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

The present invention also pertains to kits useful in the methods of the invention. Such kits comprise components useful in any of the methods described herein, including for example, hybridization probes or primers (e.g., labeled probes or primers), reagents for detection of labeled molecules, restriction enzymes (e.g., for RFLP analysis), antibodies, allele-specific oligonucleotides, means for amplification of a subject's nucleic acids, means for analyzing a subject's nucleic acid sequence, and instructional materials. For example, in one embodiment, the kit comprises components useful for analysis of an SNP associated with intracranial aneurysm. In a preferred embodiment of the invention, the kit comprises components for detecting one or more of the SNPs associated with intracranial aneurysm elsewhere described herein.

### Methods of Identifying a Modulator of Intracranial Aneurysm

The current invention also relates to methods of identifying compounds that modulate intracranial aneurysm. In various embodiments, the method of identifying of the invention identifies a modulator compound that modulates the level or activity of a gene, or gene product, that is associated with an SNP that is associated with intracranial aneurysm, as compared with the same gene, or gene product, in a genome that does not comprise the SNP. In one embodiment, the method of identifying of the invention identifies a modulator compound that diminishes the level or activity of a gene, or gene product, that is associated with an SNP that is associated with intracranial aneurysm, as compared with the same gene, or gene product, in a genome that does not comprise the SNP. In another embodiment, the method of identifying of the invention identifies a modulator compound that increases the level or activity of a gene, or gene product, that is associated with an SNP that is associated with intracranial aneurysm, as compared with the same gene, or gene product, in a genome that does not comprise the SNP. The invention further comprises the modulator of intracranial aneurysm, as well as compositions comprising the modulator of intracranial aneurysm, identified by the methods described herein.

In one embodiment, the invention comprises a method of identifying a test compound as a modulator of intracranial aneurysm. Generally, the method of identifying a test compound as a modulator of intracranial aneurysm includes comparing a parameter of intracranial aneurysm in the presence of a test compound with a parameter of intracranial aneurysm in the absence of the test compound. Thus, in some embodiments, the method includes the steps of: measuring at least one parameter of intracranial aneurysm in the absence of the test compound; measuring the at least one parameter of intracranial aneurysm in the presence of the test compound; and comparing the level of the at least one parameter of intracranial aneurysm in the presence of the test compound with the level of the at least one parameter of intracranial aneurysm in the absence of the test compound; and identifying the test compound as a modulator of intracranial aneurysm when the level of the at least one parameter of intracranial aneurysm in the presence of the test compound is different than level of the at least one parameter of intracranial aneurysm in the absence of the test compound. In one embodiment, when the level of the parameter of intracranial aneurysm is higher in the presence of the test compound, the test compound is identified as an activator. In another embodiment, when the level of the parameter of intracranial aneurysm is lower in the presence of the test compound, the test compound is identified as an inhibitor.

In another embodiment, the invention comprises a method of identifying a test compound as a modulator of a gene, or gene product, that is associated with an SNP that is associated with intracranial aneurysm. Generally, the method of identifying a test compound as a modulator of a gene, or gene product, that is associated with an SNP that is associated with intracranial aneurysm, includes comparing a parameter of a gene, or gene product, in the presence of a test compound with a parameter of the gene, or gene product, in the absence of the test compound. Thus, in some embodiments, the method includes the steps of: measuring at least one parameter of the gene, or gene product, in the absence of the test compound; measuring the at least one parameter of the gene, or gene product, in the presence of the test compound; and comparing the level of the at least one parameter of the gene, or gene product, in the presence of the test compound with the level of the at least one parameter of the gene, or gene product, in the absence of the test compound; and identifying the test compound as a modulator of the gene, or gene product, when the level of the at least one parameter of the gene, or gene product, in the presence of the test compound is different than level of the at least one parameter of the gene, or gene product, in the absence of the test compound. In one embodiment, when the level of the parameter of the gene, or gene product, is higher in the presence of the test compound, the test compound is identified as an activator. In another embodiment, when the level of the parameter of the gene, or gene product, is lower in the presence of the test compound, the test compound is identified as an inhibitor. In various embodiments of the method, the measured parameter of the gene, or gene product, is at least one of: the level of expression, the level of transcription, the level of splicing, the pattern of splicing, the level of translation, or the level of activity (e.g., enzymatic, binding, etc.). In various embodiments, the gene, or gene product, that is associated with an SNP that is associated with intracranial aneurysm, is at least one of: EDNRA, SOX17, CDKN2A, CDKN2B, CNNM2, NDUFA12, NR2C1, FGD6, VEZT, KL, STARD13, RBBP8, DSTN, or RRBP1.

Suitable test compounds include, but are not limited to, a chemical compound, a polypeptide, a peptide, a peptidomemetic, an antibody, a nucleic acid, an antisense nucleic acid, an shRNA, a ribozyme, and a small molecule chemical compound. Other methods, as well as variations of the methods disclosed herein, will be apparent from the description of this invention. In various embodiments, the test compound concentration in the screening assay can be fixed or varied. A single test compound, or a plurality of test compounds, can be tested at one time.

The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem., 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical that new chemical entities with useful properties are generated by identifying a chemical compound (called a “lead compound”) with some desirable property or activity, creating variants of the lead compound, and evaluating the property and activity of those variant compounds.

In one embodiment, high throughput screening methods involve providing a library containing a large number of test compounds potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Therapeutic Modulator Compositions and Methods of Use

In various embodiments, the present invention includes modulator compositions and methods of preventing and treating intracranial aneurysm. In various embodiments, the modulator compositions and methods of treatment of the invention modulate the level or activity of a gene, or gene product, associated with an SNP that is associated with intracranial aneurysm.

It will be understood by one skilled in the art, based upon the disclosure provided herein, that modulating a gene, or gene product, encompasses modulating the level or activity of a gene, or gene product, associated with an SNP that is associated with intracranial aneurysm, including, but not limited to, transcription, translation, splicing, enzymatic activity, binding activity, or combinations thereof, Thus, modulating the level or activity of a gene, or gene product, associated with an SNP that is associated with intracranial aneurysm includes, but is not limited to, modulating transcription, translation, splicing, or combinations thereof, of a nucleic acid; and it also includes modulating any activity of polypeptide gene product as well.

In various embodiments, the modulated gene, or gene product, that is associated with an SNP that is associated with intracranial aneurysm, is at least one of: EDNRA, SOX17, CDKN2A, CDKN2B, CNNM2, NDUFA12, NR2C1, FGD6, VEZT, KL, STARD13, RBBP8, DSTN, or RRBP1.

Modulation of a gene, or gene product, can be assessed using a wide variety of methods, including those disclosed herein, as well as methods known in the art or to be developed in the future. That is, the routineer would appreciate, based upon the disclosure provided herein, that modulating the level or activity of a gene, or gene product, can be readily assessed using methods that assess the level of a nucleic acid encoding a gene product (e.g., mRNA), the level of polypeptide gene product present in a biological sample, the activity of polypeptide gene product present in a biological sample, or combinations thereof.

One skilled in the art, based upon the disclosure provided herein, would understand that the invention is useful in treating intracranial aneurysm, or a pathology associated with intracranial aneurysm, in a subject in need thereof, whether or not the subject also being treated with other medication or therapy. Further, the skilled artisan would further appreciate, based upon the teachings provided herein, that the pathologies associated with intracranial aneurysm treatable by the compositions and methods described herein encompass any pathology associated with intracranial aneurysm.

The modulator compositions and methods of the invention that modulate the level or activity of a gene, or gene product, associated with an SNP that is associated with intracranial aneurysm, include, but should not be construed as being limited to, a chemical compound, a protein, a peptide, a peptidomemetic, an antibody, a ribozyme, a small molecule chemical compound, an antisense nucleic acid molecule (e.g., siRNA, miRNA, etc.), or combinations thereof. One of skill in the art would readily appreciate, based on the disclosure provided herein, that a modulator composition encompasses a chemical compound that modulates the level or activity of a gene, or gene product, associated with intracranial aneurysm. Additionally, a modulator composition encompasses a chemically modified compound, and derivatives, as is well known to one of skill in the chemical arts.

The modulator compositions and methods of the invention include antibodies. The antibodies of the invention include a variety of forms of antibodies including, for example, polyclonal antibodies, monoclonal antibodies, intracellular antibodies (“intrabodies”), Fv, Fab and F(ab)₂, single chain antibodies (scFv), heavy chain antibodies (such as camelid antibodies), synthetic antibodies, chimeric antibodies, and humanized antibodies. In one embodiment, the antibody of the invention is an antibody that specifically binds to a polypeptide gene product of a gene associated with an SNP that is associated with intracranial aneurysm. In another embodiment, the antibody of the invention is an antibody that specifically binds to molecule that interacts with a polypeptide gene product of a gene associated with intracranial aneurysm.

Modulators known in art useful in the compositions and methods of the invention described herein include modulators such as, sitaxentan, ambrisentan, atrasentan, BQ-123, bosentan, tezosentan, and derivatives thereof.

Further, one of skill in the art would, when equipped with this disclosure and the methods exemplified herein, appreciate that modulators include such modulators as discovered in the future, as can be identified by well-known criteria in the art of pharmacology, such as the physiological results of modulation of the genes, and gene products, as described in detail herein and/or as known in the art. Therefore, the present invention is not limited in any way to any particular modulator composition as exemplified or disclosed herein; rather, the invention encompasses those modulator compositions that would be understood by the routineer to be useful as are known in the art and as are discovered in the future.

Further methods of identifying and producing modulator compositions are well known to those of ordinary skill in the art, including, but not limited, obtaining a modulator from a naturally occurring source (i.e., Streptomyces sp., Pseudomonas sp., Stylotella aurantium). Alternatively, a modulator can be synthesized chemically. Further, the routineer would appreciate, based upon the teachings provided herein, that a modulator composition can be obtained from a recombinant organism. Compositions and methods for chemically synthesizing modulators and for obtaining them from natural sources are well known in the art and are described in the art.

One of skill in the art will appreciate that a modulator can be administered as a small molecule chemical, a polypeptide, a peptide, an antibody, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is modulator of a gene, or gene product, associated with an SNP that is associated with intracranial aneurysm. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

Antisense oligonucleotides are DNA or RNA molecules that are complementary to some portion of an RNA molecule. When present in a cell, antisense oligonucleotides hybridize to an existing RNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to modulate the amount of a gene, or gene product, associated with intracranial aneurysm, thereby modulating the amount or activity of the gene product.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expression can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn, 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that the modulators of the invention can be administered singly or in any combination. Further, the modulators of the invention can be administered singly or in any combination in a temporal sense, in that they may be administered concurrently, or before, and/or after each other. One of ordinary skill in the art will appreciate, based on the disclosure provided herein, that the modulator compositions of the invention can be used to prevent or to treat intracranial aneurysm, and that a modulator composition can be used alone or in any combination with another modulator to effect a therapeutic result. In various embodiments, any of the modulators of the invention described herein can be administered alone or in combination with other modulators of other molecules associated with intracranial aneurysm.

It will be appreciated by one of skill in the art, when armed with the present disclosure including the methods detailed herein, that the invention is not limited to treatment of intracranial aneurysm, or a pathology associated with intracranial aneurysm, that is already established. Particularly, the disease, disorder or pathology need not have manifested to the point of detriment to the subject; indeed, the disease, disorder or pathology need not be detected in a subject before treatment is administered. That is, significant signs or symptoms of intracranial aneurysm do not have to occur before the present invention may provide benefit. Therefore, the present invention includes a method for preventing intracranial aneurysm, in that a modulator composition, as discussed previously elsewhere herein, can be administered to a subject prior to the onset of the disease or disorder, thereby preventing the disease or disorder. The preventive methods described herein also include the treatment of a subject that is in remission for the prevention of a recurrence of intracranial aneurysm, or a pathology associated with intracranial aneurysm.

One of skill in the art, when armed with the disclosure herein, would appreciate that the prevention of intracranial aneurysm, or a pathology associated with intracranial aneurysm, encompasses administering to a subject a modulator composition as a preventative measure against the development of, or progression of, intracranial aneurysm, or a pathology associated with intracranial aneurysm. As more fully discussed elsewhere herein, methods of modulating the level or activity of a gene, or gene product, associated with an SNP that is associated with intracranial aneurysm, encompass a wide plethora of techniques for modulating not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases, disorders and pathologies where modulating the level or activity of a gene, or gene product, that is associated with an SNP associated with intracranial aneurysm, mediates, treats or prevents the disease, disorder or pathology. Various methods for assessing whether a disease relates to a gene, or gene product, that is associated with an SNP associated with intracranial aneurysm are described elsewhere herein and are known in the art. Further, the invention encompasses treatment or prevention of such diseases discovered in the future.

The invention encompasses administration of a modulator of a gene, or gene product, that is associated with an SNP associated with intracranial aneurysm, to practice the methods of the invention; the skilled artisan would understand, based on the disclosure provided herein, how to formulate and administer the appropriate modulator composition to a subject. Indeed, the successful administration of the modulator has been reduced to practice as exemplified herein, However, the present invention is not limited to any particular method of administration or treatment regimen.

### Pharmaceutical Compositions

Compositions identified as potentially useful modulator compounds for treatment and/or prevention of intracranial aneurysm, can be formulated and administered to a subject for treatment and/or prevention of intracranial aneurysm, as now described.

The invention encompasses the preparation and use of pharmaceutical compositions comprising a composition useful for treatment of intracranial aneurysm, disclosed herein as modulator of a gene, or gene product, that is associated with an SNP that is associated with intracranial aneurysm. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate modulator may be combined and which, following the combination, can be used to administer the appropriate modulator thereof, to a subject.

The pharmaceutical compositions useful for practicing the invention may be administered to deliver a dose of between about 0.1 ng/kg/day and 100 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic coin position, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose, Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin, Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations,

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution, This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device.

Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials, Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Common Variant Near the Endothelin Receptor Type a (EDNRA) Gene is Associated with Intracranial Aneurysm Risk

The pathogenesis of intracranial aneurysm (IA) formation and rupture is complex, with significant contribution from genetic factors. Previously reported genome-wide association studies based on European discovery and Japanese replication cohorts of 5,891 cases and 14,181 controls have identified five disease-related loci. These studies were based on testing replication of genomic regions that contained SNPs with posterior probability of association (PPA) greater than 0.5 in the discovery cohort. To identify additional intracranial aneurysm risk loci, 14 loci with PPAs in the discovery cohort between 0.1 and 0.5 were pursued. Twenty-five SNPs from these loci were genotyped using two independent Japanese cohorts, and the results from discovery and replication cohorts were combined by meta-analysis. The results demonstrated significant association of intracranial aneurysm with rs6841581 on chromosome 4q31.23, immediately 5′ of the endothelin receptor type A with P=2.2×10⁻⁸ [odds ratio (OR)=1.22, PPA=0.986]. Substantially increased evidence of association for two other regions on chromosomes 12q22 (OR=1.16, P=1.1×10⁻⁷, PPA=0.934) and 20p12.1 (OR=1.20, P=6.9×10⁻⁷, PPA=0.728) was also observed. Although endothelin signaling has been hypothesized to play a role in various cardiovascular disorders, results presented herein are unique in providing genetic evidence for a significant association with intracranial aneurysm and suggest that manipulation of the endothelin pathway may have important implications for the prevention and treatment of IA.

Previous studies used a discovery cohort of 2,780 cases and 12,515 controls, 831,532 genotyped and imputed autosomal SNPs to discover associations with IA. Bayesian measure of the strength of association—the posterior probability of association (PPA)—was used to prioritize SNPs by calculating to what extent the data supports association with intracranial aneurysm (Yasuno et al., 2010, Nat Genet, 42:420-425). This analysis revealed five loci with PPA>0.5. Following replication genotyping using two independent Japanese cohorts and combining the discovery and replication cohort results, all five loci surpassed the genome-wide significance level of 5×10⁻⁸ (observed P values≦2.5×10⁻⁹) with PPA≧0.998, suggesting that each locus contains a variant that confers risk of developing IA. The five loci were on chromosomes 8q12.1 (SOX17), 9p21.3 (CDKN2A/CDKN2B), 10q24.32 (CNNM2), 13q13.1 (KL/STARD13), and 18q11.2 (RBBP8) (Yasuno et al., 2010, Nat Genet, 42:420-425).

Because these five loci explained only ˜5% of the intracranial aneurysm genetic risk and the number of SNPs showing P values<0.001 was greater than that expected by chance alone (Yasuno et al., 2010, Nat Genet, 42:420-425), it is likely that there is a presence of additional true intracranial aneurysm risk loci among a range of SNPs showing weaker evidence of association in the discovery cohort. The examination for additional true intracranial aneurysm risk loci, using the two Japanese replication cohorts, is described herein.

The materials and methods used in the experiments are now described.

Ethics

The study protocol was approved by the Yale Human Investigation Committee (HIC protocol #7680). Institutional review board approval for genetic studies, along with written consent from all study participants, was obtained at all participating institutions.

Study Subjects

Consent was obtained from all study participants. In all cases, diagnosis of intracranial aneurysm (IA) was made either with computerized tomography (CT) angiogram, magnetic resonance (MR) angiogram, or cerebral digital subtraction angiogram and confirmed at surgery, when applicable. Rupture of an aneurysm was defined as identification of acute subarachnoid hemorrhage (SAH), as evident on CT or MR imaging, from a proven aneurysm. Subjects with SAH without saccular IA, nonsaccular intracranial aneurysm (i.e., fusiform and dissection aneurysms), and those with known genetic syndromes that are believed to predispose to intracranial aneurysm (i.e., polycystic kidney disease and Ehlers-Danlos syndrome Type IV) were excluded from the study.

Discovery and Replication Cohorts

The discovery case-control samples comprised a genetically and sex-matched Finnish (FI) cohort of 808 cases and 4,393 controls, and combined European (CE) cohort of 1,972 cases and 8,122 controls, The latter cohort consisted of three subcohorts based on the centers that ascertained the case samples: the Netherlands (NL), Germany (DE), and a pan-European (AN: @neurIST) cohort. The replication cohorts included two independent Japanese case-control samples (JP1 and JP2). JP1 consisted of 829 cases and 761 controls; JP2 consisted of 2,282 cases and 905 controls. These cohorts were described in detail elsewhere (Yasuno et al., 2010, Nat Genet 42:420-425).

Replication Strategy

A two-stage design was used to follow-up 14 candidate intervals. A two-stage design was used to confirm association signals for loci that showed posterior probability of association (PPA) values between 0.1 and 0.5 in the discovery cohort (Yasuno et al., 2010, Nat Genet 42:420-425). First, the SNP with the maximum PPA within each region was selected as the candidate for replication genotyping. If this was an imputed SNP and a genotyped SNP with a similar PPA was found as an alternative, the genotyped one was selected. For each region, if a second SNP that was highly correlated with the one selected above was available, it was also genotyped to assure the genotyping quality. In the first stage, all of the candidate regions were analyzed using the larger JP2 cohort (Table 1 and Table 4). For the second stage, the SNPs that showed Bayes factor (BF)>0.5 in the JP2 cohort with the same risk allele as the discovery cohort were chosen and genotyped using the JP1 cohort.

Genotyping and Quality Control

For SNPs reported in Table 1 and Table 4, genotyping of the JP1 cohort was performed using either the MassARRAY (Sequenom) assay or the Taqman (Applied Biosystems) platform. JP2 cases were genotyped using the multiplex PCR-based Invader assay (Third Wave Technologies), and JP2 controls were genotyped using the Illumina platform (Kamatani et al., 2009, Nat Genet 41:591-595). SNPs were excluded if any of the following three conditions were met in either cases or controls: (i) fraction of missing genotypes>0.1; (ii) P value of the exact test of Hardy-Weinberg equilibrium<0.001; or (iii) minor allele frequency<0.01.

Statistical Analysis

Association between each SNP and intracranial aneurysm was tested by fitting a logistic regression model with an additive effect of allele dosage and sex as a covariate. For each SNP, a P value was obtained from the score test (two-sided) and the logarithm of per-allele odds ratio (OR) with SE was estimated by maximizing the likelihood. For multilocus analysis, genotypes from JP1 and JP2 were combined incorporating the cohort label into the above model, and the discovery cohort was analyzed using the conditional logistic regression as described previously (Yasuno et al., 2010, Nat Genet 42:420-425).

Meta-analysis was performed to combine the cohort-wise results. Primary analysis was based on the fixed-effects model (JP1+JP2 for replication, FI+CE+JP1+JP2 for a combined result). To assess the heterogeneity of ORs between cohorts, CE was first divided into three cohorts (NL, DE, and AN; see above), aiming to analyze data without averaging ORs over the European subcohorts, and then six cohorts were combined (i.e., FI+NL+DE+AN+JP1+JP2) using the random-effects model. The restricted maximum-likelihood procedure was used to estimate the inter-cohort heterogeneity variance (τ2), from which Cochran's Q statistic and 12 statistic was calculated, using R-function MiMa (Viechtbauer, 2005, J Educ Behav Stat, 30:261-293).

Evaluating the Strength of Association

Besides calculating the test P values, the strength of association using the BF and PPA was also quantitatively measured, which provided a probabilistic measure of the strength of the evidence (Stephens et al., 2009, Nat Rev Genet, 10:681-690). The BF is the ratio of the probabilities of the data under the alternative hypothesis versus the null hypothesis, which can be interpreted as the fold-change of the odds of association before and after observing the data. For computational simplicity, BF was approximated following Wakefield (Wakefield, 2007, Am J Hum Genet, 81:208-227). For the prior distribution of the log-OR of every SNP, a normal distribution with a mean of O and SD of log(1.5)/Φ-1(0.975) was assumed, where Φ is the normal distribution function (Wellcome Trust Case Control Consortium, 2007, Nature, 447: 661-678). The association between a SNP and intracranial aneurysm was regarded as replicated if BF>10 in the replication cohort (i.e., 10-fold increase in the odds of association after observing replication data) (Yasuno et al., 2010, Nat Genet 42:420-425). A uniform prior probability of association of 0.0001 was assumed across all of the SNPs (Yasuno et al., 2010, Nat Genet 42:420-425).

Two-Locus Interaction

Deviation from a linear model, in which two SNPs combine to increase the log-odds of disease in an additive fashion, was tested for by fitting a model with an interaction term between two SNPs in addition to linear terms. The interaction OR and 95% CI were obtained from the maximum-likelihood estimate and the interaction P value was obtained from the Wald test.

Cumulative Effect

Potential clinical implications of the genetic profiles of the intracranial aneurysm risk loci were evaluated following the approach described by Clayton (Clayton, 2009, PLoS Genet, 5:e1000540). A model was first fitted with additive effects of seven loci. [rs9298506, rs1333040, rs12413409, rs9315204, and rs11661542 from the previous report (Yasuno et al., 2010, Nat Genet 42:420-425), and rs6841581 and rs6538595 from the present study] and then the risk scores were calculated for every individual using the estimated log-ORs for the seven SNPs and the individual's genotypes. The receiver-operating characteristic curve was depicted for each ethnic cohort (FI, CE, and JP) by calculating the proportions of cases and controls with risk scores exceeding each possible value, The sibling recurrence risk because of the seven SNPs was estimated by assuming the polygenic model that fits well to the data (Clayton, 2009, PLoS Genet, 5:e1000540). The fraction of the sibling recurrence risk attributable to the seven loci was calculated by taking the ratio of the logarithm of this value and an epidemiologically estimated value of 4 (Schievink, 1997, Neurosurgery, 40:651-663). The ratio of the exponential of the mean of the risk scores for control subjects within the top versus bottom 5% or 1% were also calculated to obtain approximated ORs of disease between these classes.

The results of the experiments are now described.

### Analysis of Previously Uninvestigated Intervals.

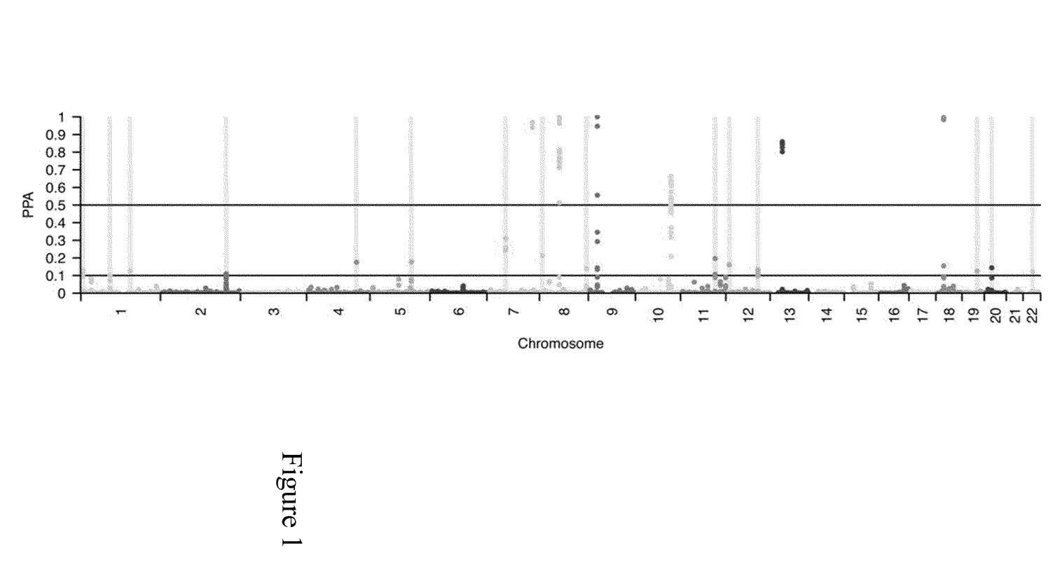

The statistical analysis of the discovery cohort was described in detail previously (Yasuno et al., 2010, Nat Genet, 42:420-425). Following strict sample and SNP quality control (QC) measures, cases and controls of the same sex were matched based on inferred genetic ancestry to eliminate potential confounding because of population stratification and sex. Association of 831,529 QC-passed SNPs with intracranial aneurysm were then tested for and the strength of the association was evaluated using PPA (Yasuno et al., 2010, Nat Genet, 42:420-425). In addition to the five previously investigated loci with PPA>0.5, there were 15 additional loci with PPA>0.1 (observed values between 0.1 and 0.31) (FIG. 1 and Table 3). One of these intervals, on chromosome 7 (PPA=0.31), was detected only by imputed SNPs without support from nearby genotyped SNPs in linkage disequilibrium (LD), suggesting an imputation error (Wellcome Trust Case Control Consortium, 2007, Nature, 447:661-678). Therefore, the two-stage follow-up genotyping was pursued for the remaining 14 loci (Table 1).

TABLE 1
Cohort-wise association test results for 14 representative SNPs

| | | | Discovery | | | JP2 (Replication 1) | | | JP1 (Replication 2) | | |
|---|---|---|---|---|---|---|---|---|---|---|---|
| Chromosome | SNP | Position | RA | P | PPA | RA | P | log₁₀ (BF) | RA | P | log₁₀ (BF) |
| 1p36.31 | rs1876848 | 6,876,262 | G | 2.0E−05 | 0.1128 | A | 0.016 | 0.61 | NA | | |
| 1p22.2 | rs1725390 | 91,031,160 | A | 2.0E−05 | 0.1011 | A | 0.14 | −0.11 | A | 0.59 | −0.40 |
| 1q21.3 | rs905938 | 153,258,013 | T | 1.7E−05 | 0.1252 | T | 0.62 | −0.18 | T | 0.82 | −0.15 |
| 2q33.1* | rs787994 | 197,931,366 | T | 2.1E−05 | 0.0988 | C | 8.6E−05 | 2.51 | T | 0.070 | 0.19 |
| 4q31.23 | rs6841581 | 148,620,640 | G | 1.1E−05 | 0.1750 | G | 0.0066 | 0.93 | G | 0.023 | 0.53 |
| 5q23.2 | rs2287696 | 122,488,231 | A | 1.1E−05 | 0.1760 | A | 0.27 | −0.32 | NA | | |
| 8p23.2 | rs2045637 | 2,963,188 | A | 8.6E−06 | 0.2139 | G | 0.36 | −0.38 | NA | | |
| 8q24.23* | rs1554349 | 139,604,536 | A | 7.9E−05 | 0.0349 | A | 0.15 | −0.12 | G | 0.33 | −0.28 |
| 11q22.2 | rs2124216 | 101,644,113 | A | 9.1E−06 | 0.1963 | A | 0.20 | −0.24 | G | 0.65 | −0.43 |
| 12p13.31 | rs728342 | 5,577,633 | G | 1.2E−05 | 0.1601 | G | 0.33 | −0.30 | G | 0.56 | −0.33 |
| 12q22 | rs6538595 | 94,030,754 | A | 1.8E−05 | 0.1136 | A | 0.0051 | 1.01 | A | 0.13 | −0.012 |
| 19q13.12 | rs1688005 | 40,340,205 | G | 1.6E−05 | 0.1244 | T | 0.16 | −0.069 | NA | | |
| 20p12.1 | rs1132274 | 17,544,155 | A | 1.5E−05 | 0.1435 | A | 0.012 | 0.69 | NA^{†} | | |
| 22q12.1 | rs133885 | 24,489,289 | G | 1.6E−05 | 0.1230 | G | 0.67 | −0.42 | NA | | |

Genomic positions were based on the human genome build 36.
NA, the SNP was not genotyped
RA, risk allele aligned to the forward strand of the reference genome.
*For these loci, the lead SNPs were not genotyped (see Methods).
^{†}Genotyping of rs1132274 in the JP1 cohort failed.

For the first stage, 25 SNPs located within these 14 intervals were genotyped using the larger of the two Japanese cohorts (JP2), comprising 2,282 cases and 905 controls (Table 4). All of these SNPs passed QC filters. Association tests revealed that three of these loci, on chromosomes 4q31.23, 12q22, and 20p12.1, supported association with intracranial aneurysm [i.e., Bayes Factor (BF)>1](Table 1). Although the data also supported association with intracranial aneurysm at SNPs on chromosomes 1p36.31 and 2q33.1, the risk alleles were different from those found in the discovery cohort (Table I). Further study of these latter loci will be needed to determine whether this might be because of allelic heterogeneity between European and East Asian populations.

In the second stage, using the JP1 cohort, 13 SNPs in a total of nine loci that showed BF>0.5 in the JP2 cohort with the same risk allele as the discovery cohort were genotyped (Table 1 and Table 4). Two of the genotyped SNPs (rs2282652 and rs1132274) failed to pass the QC filters and were excluded from further analysis, leading to coverage of eight of the nine loci, JP1 data supported association with intracranial aneurysm at SNPs located in two intervals, 4q31.23 and 12q22 (Table 1 and Table 4).

Meta-analysis of JP1 and JP2 cohorts revealed that the combined replication data strengthened the association with intracranial aneurysm at SNPs located within two of the eight loci on chromosomes 4q31.23 and 12q22 (rs6841581 and rs6538595, respectively) by increasing the odds of association 85.1- and 24.0-fold, respectively (P=0.00042 and 0.0017) (Table 2 and Table 4). One more SNP, rs1132274, for which the JP1 cohort data were not available, showed P=0.021 and BF=4.9 in the JP2 cohort.

TABLE 2
Summary of results for SNPs located in three unique genomic intervals on
chromosomes 4q31.23, 12q22, and 20p12.1

| Chromosome | SNP | Position | Gene | RA | Cohort | P value | log₁₀ (BF) | PPA | OR (95% CI) |
|---|---|---|---|---|---|---|---|---|---|
| 4q31.23 | rs6841581 | 148,620,640 | EDNRA | G | Discovery | 1.1E−05 | 3.33 | 0.1750 | 1.25 (1.13-1.39) |
| | | | | G | Replication | 0.00042 | 1.93 | | 1.20 (1.08-1.32) |
| | | | | | Combined | 2.2E−08 | 5.84 | 0.9857 | 1.22 (1.14-1.31) |
| 12q22 | rs6538595 | 94,030,754 | NDUFA12/NR2C1/ | A | Discovery | 1.8E−05 | 3.11 | 0.1136 | 1.16 (1.08-1.24) |
| | | | FGD6/VEZT | | | | | | |
| | | | | A | Replication | 0.0017 | 1.38 | | 1.16 (1.06-1.28) |
| | | | | | Combined | 1.1E−07 | 5.15 | 0.9343 | 1.16 (1.10-1.23) |
| 20p12.1 | rs1132274 | 17,544,155 | RRBP1 | A | Discovery | 1.5E−05 | 3.22 | 0.1435 | 1.22 (1.11-1.33) |
| | | | | A | Replication (JP2) | 0.012 | 0.69 | | 1.16 (1.03-1.30) |
| | | | | | Combined | 6.9E−07 | 4.43 | 0.7279 | 1.20 (1.11-1.28) |

Genomic positions were based on the human genome build 36.
OR, the per-allele odds ratio of the risk allele;
RA, risk allele aligned to the forward strand of the reference genome.
Replication and combined results are based on the fixed-effects model.

### Combined Results.

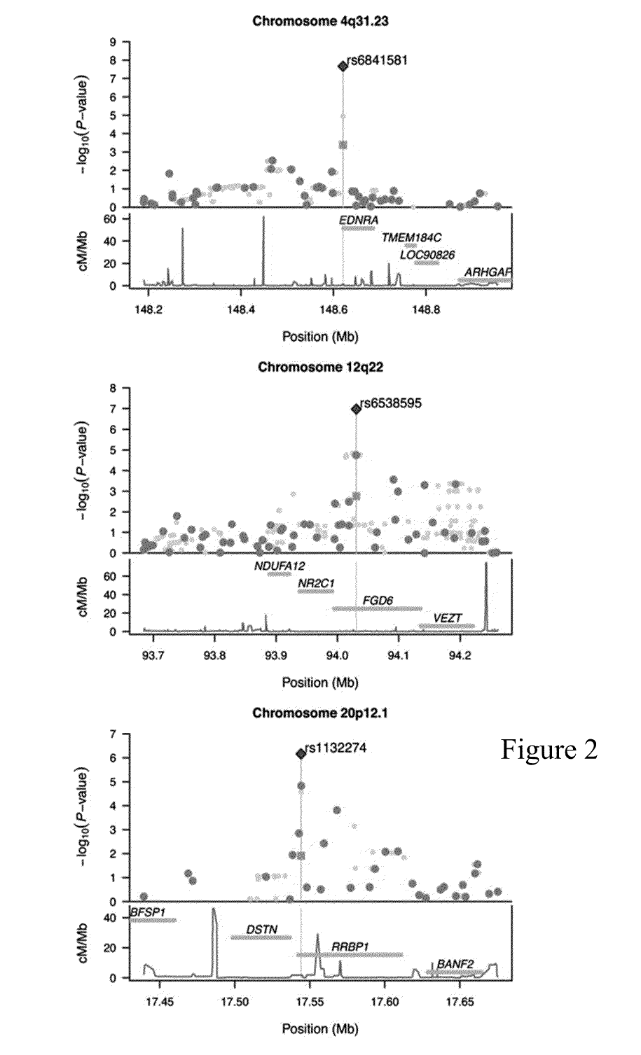

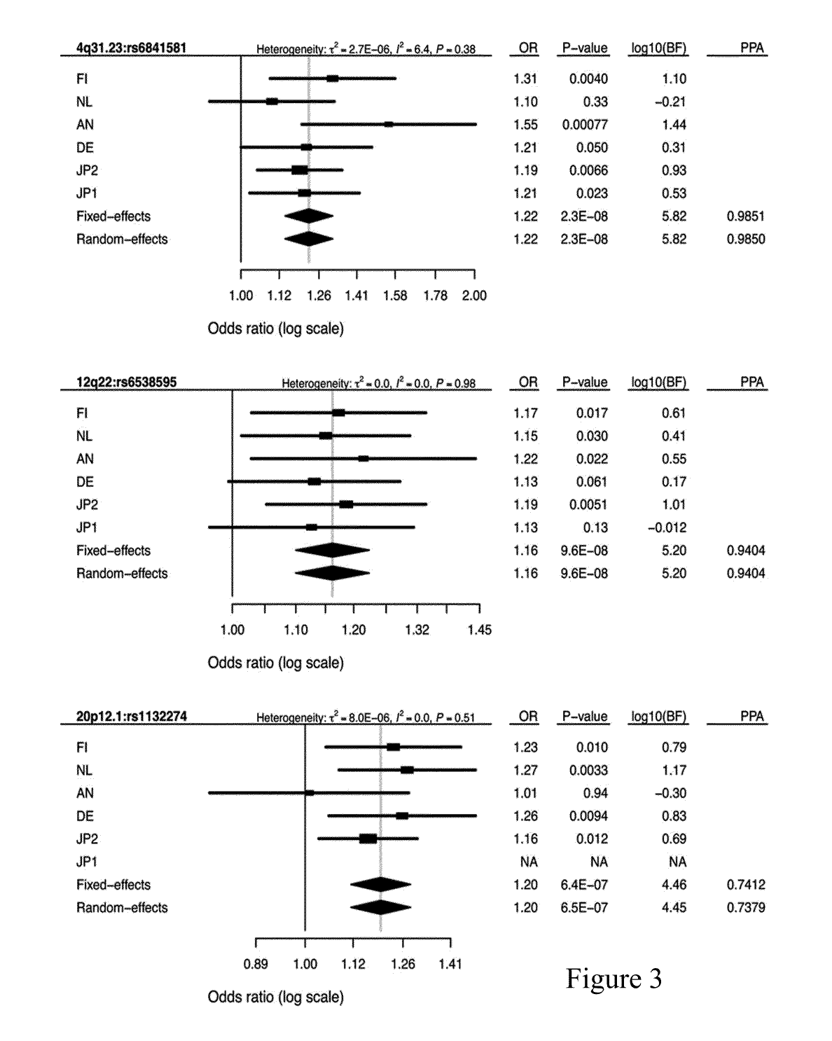

Meta-analysis was performed to combine the results from the discovery and replication cohorts (FIG. 2 and Table 4). The new genotyping results substantially increased the strength of the evidence of association for three loci on chromosomes 4q31.23, 12q22, and 20p12.1, compared with the discovery data (FIG. 2, FIG. 3, Table 2, and Table 4).

The strongest association was detected at rs6841581, located at 148,620,640 base pairs on chromosome 4q31.23, with P=2.2×10⁻⁸ [odds ratio (OR)=1.22, PPA=0.986](FIG. 2 and FIG. 3). Only a single gene, Endothelin Receptor Type A (EDNRA), is located within the LD interval containing this SNP (FIG. 2). SNP rs6841581 lies only 1,129 bases from the 5′ end of the EDNRA transcript (NM_{—}001957.3), located within an interval predicted to have regulatory functions (University of California at Santa Cruz genome browser, www.genome.ucsc.edu). The encoded protein, EDNRA, plays an important role in the cerebrovascular physiology. An examination of the publicly available eQTL databases (eqtl.uchicago.edu) did not reveal any SNPs that significantly alter EDNRA expression.

The second strongest association was at rs6538595 on chromosome 12q22 (OR=1.16, P=1.1×10⁻⁷, PPA=0.934) (FIG. 2 and FIG. 3). A cluster of SNPs strongly correlated with rs6538595 is associated with intracranial aneurysm and is mapped within the introns of the FYVE, RhoGEF, and PH domain-containing 6 (FGD6) gene (FIG. 2). Three other genes, NADH dehydrogenase (ubiquinone) 1 alpha subcomplex 12 (NDUFA12), nuclear receptor subfamily 2, group C, member 1 (NR2C1), and Vezatin, Adherens Junctions Transmembrane (VEZT), are located within the same LD interval as rs6538595.

Finally, although the JP1 cohort data were not available, a missense variant, rs1132274, within the Ribosome Binding Protein 1 (RRBP1) gene located on chromosome 20p12.1 showed moderate evidence of association with intracranial aneurysm (OR=1.20, P=6.9×10⁻⁷, PPA=0.728) (FIG. 2 and FIG. 3). Another gene, Destrin (DSTN), is also contained in the same LD interval.

There was no evidence for a two-locus interaction that was consistent across all cohorts between various SNPs that were found to be associated with intracranial aneurysm (Table 5).

### Cumulative Effect

Analysis of the cumulative effect of the seven intracranial aneurysm risk loci including the two SNPs (rs6841581 and rs6538595) replicated here, as well as the previously identified five SNPs, explains 6.1%, 4.4%, and 4.1% of the familial risk in the Finnish, European, and Japanese cohorts, respectively (Table 6). The ORs of developing intracranial aneurysm between the top and bottom 1% risk groups representing the tails of the distribution of genetic profiles ranged between 5.74 and 8.39 for the Japanese, European, and Finnish cohorts analyzed (Table 6).

### Endothelin System and IA

In this study, the association of SNPs located within three intervals on chromosomes 4q31.23, 12q22, and 20p12.1 with intracranial aneurysm is demonstrated. Among these, rs6841581, located on chromosome 4q31.23 near the ENDRA gene, showed the most significant association, with a P value of 2.2×10⁻⁸ (PPA=0.986). The data for another SNP, rs6538595, on chromosome 12q22 also supported association with IA; replication data increased the probability of association from 0.114 to 0.934 (P=1.1×10⁻⁷). The evidence of association for the third SNP, rs1132274, on chromosome 20p12.1 was less. Although the replication data from the JP2 cohort increased the support for association with IA, the JP1 cohort could not reliably genotype using the available platforms, thereby limiting the evidence for replication at this locus to a single Japanese cohort. Thus, only rs6841581 and rs6538595 on chromosomes 4q31.23 and 12q22, respectively, are considered as previously undetected risk loci for IA. Although the addition of these two loci remarkably increased the difference in the odds of disease between the highest and lowest risk groups to 5.7- and 8.4-fold in Japanese and Finnish cohorts, respectively, this only slightly improved the predictability of the disease risk (Table 6).

The most significant locus, 4q31.23, contains a single gene, EDNRA, which has been of great interest in various cardiovascular pathologies. Indeed, the endothelin system has been implicated in the pathogenesis of cardiovascular disorders, including pulmonary and primary hypertension (Humbert et al., 2004, N Engl J Med, 351:1425-1436). EDNRA, along with EDNRB, are G protein-coupled receptors for endothelins, with the 21-aa endothelin-1 (EDN1) being the predominant isoform (Yanagisawa et al., 1988, Nature, 332:411-415; Inoue et al., 1989, Proc Natl Acad Sci USA, 86:2863-2867). EDN1 is produced primarily by the vascular endothelium and smooth-muscle cells and is involved in maintaining vasomotor control and vascular homeostasis (Yanagisawa et al., 1988, Nature, 332:411-415), EDNRA is found on vascular smooth-muscle cells, including the cerebrovasculature, along with the heart, kidney, and neuronal cells (Yu et al., 1995, Br J Pharmacol, 116:2441-2446), and mediates the vasoconstriction and mitogenic effects of EDN1 (Alberts et al., 1994, J Biol Chem, 269:10112-10118; Suzuki et al., 1999, Circ Res, 84:611-619). On the other hand, EDNRB reside on both smooth-muscle and endothelial cells, with downstream signaling through nitric oxide (Miyauchi et al., 1999, Annu Rev Physiol, 61:391-415). Thus, EDN1 and its receptors, EDNRA and EDNRB, play key roles in the maintenance of the vasculature by controlling the balance between vasoconstriction and vasodilation in response to hemodynamic stress.

Although not wishing to be bound by any particular theory, EDNRA may play a role in intracranial aneurysm pathogenesis in two distinct ways, depending on whether the EDNRA-mediated signaling is up- or down-regulated by the causal variant captured by the established SNP rs6841581. While not wishing to be held to any particular theory, increased EDNRA-mediated signaling might predispose to progression and rupture of intracranial aneurysm through a mechanism analogous to the one that has been implicated for the development of atherosclerosis (Kowala et al, 1995, Am J Pathol, 146:819-826). Endothelin signaling has been shown to be activated at the site of vascular injury (Wang et al., 1996, Circ Res, 78:322-328). Immediately after the injury, this might be beneficial with recruitment of endothelial cells to repair the damage (Douglas et al., 1993, J Cardiovasc Pharmacol, 22(Suppl 8):S371-S373). Following this initial response, however, prolonged, excessive endothelin signaling might be harmful by leading to atherosclerosis. EDNRA mediates this vascular mitogenic effect of EDN1 by promoting cell cycle progression and proliferation, both of which might play a role in intracranial aneurysm progression and rupture (Alberts et al., 1994, J Biol Chem, 269:10112-10118; Suzuki et al., 1999, Circ Res, 84:611-619; Lerman et al., 1991, N Engl J Med, 325:997-1001). Consistent with this role of endothelins, increased EDN1 and EDNRA levels have been reported following rupture of intracranial aneurysms (Suzuki et al., 1990, Ann Med, 22:233-236; Kraus et al., 1991, Surg Neurol, 35:20-29; Itoh et al., 1994, J Neurosurg, 81:759-764). In addition, hypertension and smoking, both well-established risk factors of intracranial aneurysm pathogenesis, have been shown to alter the expression of endothelins (Xu et al., 2010, Pharmacol Ther, 127:148-155).

Without wishing to be held to any particular theory, if EDNRA-mediated signaling were attenuated, the risk allele might predispose to the formation of intracranial aneurysm because of the failure of the repair mechanism mentioned above (Wang et al., 1996, Circ Res, 78:322-328; Douglas et al., 1993, J Cardiovasc Pharmacol, 22(Suppl 8):S371-S373), Decreased signaling might interfere with the repair process in response to vascular injury, limiting the recruitment of vascular progenitor cells to the site of the damage with resultant defective repair, which in turn might result in the formation of arterial aneurysms. In support of this hypothesis, the use of at least one EDNRA antagonist in primates has been shown to be associated with the formation of preaneurysmal coronary artery lesions, characterized by fragmentation of the internal elastic lamina and loss of the medial smooth muscle (Stephan-Gueldner et al., 2000, Toxicol Lett, 112-113:531-535).

Finally, endothelins, specifically EDNRA-mediated signaling, have been implicated in the pathogenesis of cerebral vasospasm, the pathologic vasoconstriction of the cerebral blood vessels that can often result in delayed ischemic neurologic deficits following intracranial aneurysm rupture (Itoh et al., 1994, J Neurosurg, 81:759-764; Asano et al., 1989, Biochem Biophys Res Commun, 159:1345-1351). Several animal studies suggested a beneficial effect of EDNRA inhibition in treating vasospasm, leading to human clinical trials (Nirei et al, 1993, Life Sci, 52:1869-1874). The use of clazosentan, a selective EDNRA antagonist with a predilection for the central nervous system, has demonstrated reduction in angiographically demonstrated vasospasm, even though there was no improvement in clinical outcome (Macdonald et al., 2008, Stroke, 39:3015-3021; Kramer et al., 2009, Stroke, 2009, 40:3403-3406). Given the potentially biphasic role of endothelin signaling in intracranial aneurysm pathogenesis, therapeutic strategies involving EDNRA have to be considered cautiously. As mentioned elsewhere herein, the use of at least one EDNRA antagonist in primates has been shown to be associated with the formation of preaneurysmal coronary artery lesions (Stephan-Gueldner et al., 2000, Toxicol Lett, 112-113:531-535).

The discovery of a significant association of intracranial aneurysm with a risk allele at immediate proximity to EDNRA within a predicted regulatory region is unique in providing genetic evidence linking endothelins to intracranial aneurysm pathogenesis. This understanding supports pharmacological interventions that have therapeutic value in the treatment of aneurysms before rupture.

TABLE 3
Distribution of posterior probabilities of association (PPA) and effect sizes:
For each of the given intervals of PPA, the number of SNPs, observed ranges
of PPAs, per-allele ORs and P-values, and chromosomal locations are shown.

| | | Observed range | | | |
|---|---|---|---|---|---|
| | Num | | Per-allele | | |
| PPA | SNPs | PPA | OR | P-value | Chromosome |
| (0, 0.001] | 826,800 | 1.6E−05-0.0010 | 1.000-1.428 | 1.0-3.5E−03 | 1-22 |
| (0.001, 0.01] | 4,011 | 0.0010-0.0099 | 1.101-1.575 | 5.7E−03-2.0E−04 | 1-22 |
| (0.01, 0.05] | 481 | 0.0100-0.0484 | 1.129-1.527 | 3.5E−04-4.7E−05 | 1-21 |
| (0.05, 0.1] | 85 | 0.0533-0.0996 | 1.148-1.312 | 4.3E−05-2.1E−05 | 1, 2, 5, 8, 8, 9, 10, |
| | | | | | 11, 12, 15, 18, 20 |
| (0.1, 0.2] | 41 | 0.1000-0.1963 | 1.155-1.253 | 2.1E−05-9.1E−06 | 1, 2, 4, 5, 8, 9, 11, |
| | | | | | 12, 18, 19, 20, 22 |
| (0.2, 0.3] | 3 | 0.2084-0.2929 | 1.164-1.315 | 8.6E−06-4.9E−06 | 8, 9, 10 |
| (0.3, 0.4] | 4 | 0.3171-0.3714 | 1.166-1.331 | 4.3E−06-3.3E−06 | 9, 10 |
| (0.4, 0.5] | 5 | 0.4572-0.4971 | 1.365-1.369 | 2.1E−06-1.7E−06 | 10 |
| (0.5, 0.6] | 11 | 0.5048-0.5754 | 1.190-1.379 | 2.0E−06-1.2E−06 | 8, 9, 10 |
| (0.6, 0.7] | 11 | 0.6089-0.6621 | 1.376-1.381 | 1.0E−06-7.9E−07 | 10 |
| (0.7, 0.8] | 19 | 0.7119-0.7984 | 1.212-1.239 | 7.7E−07-4.6E−07 | 8 |
| (0.8, 0.9] | 8 | 0.8020-0.8597 | 1.213-1.270 | 4.5E−07-2.9E−07 | 8, 13 |
| (0.9, 0.99] | 12 | 0.9467-0.9881 | 1.198-1.264 | 9.1E−08-1.8E−08 | 8, 9, 18 |
| (0.99, 1] | 38 | 0.9901-1.0000 | 1.208-1.366 | 1.5E−08-2.2E−16 | 8, 9, 18 |

Boldface chromosomal numbers indicate that SNPs within the particular PPA ranges are studied here. Italic ones indicate that SNPs are located within the previously established intervals on chromosomes 8q11.23-q12.1, 9p21.3, 10q24.32, 13q13,1 and 18q11.2.

TABLE 4
Cohort-wise and combined association results for 25 SNPs located in 14 chromosomal intervals.

| Chr | SNP | Position | Cohort | RA | P-value | log₁₀ (BF) | PPA | OR (95% CI) |
|---|---|---|---|---|---|---|---|---|
| 1p36.31 | rs950493 | 6,867,391 | FI | T | 0.14 | −0.044 | NA | 1.12 (0.96-1.30) |
| | | | CE | T | 3.2E−05 | 2.90 | NA | 1.26 (1.13-1.41) |
| | | | NL | T | 0.0021 | 1.32 | NA | 1.30 (1.10-1.54) |
| | | | AN | T | 0.22 | −0.058 | NA | 1.16 (0.92-1.46) |
| | | | DE | T | 0.0076 | 0.89 | NA | 1.28 (1.07-1.53) |
| | | | JP2 | C | 0.019 | 0.56 | NA | 1.17 (1.03-1.33) |
| | | | JP1 | NA | NA | NA | NA | NA |
| | | | D.fxd | T | 2.4E−05 | 3.02 | 0.0949 | 1.21 (1.11-1.32) |
| | | | JP.fxd | NA | NA | NA | NA | NA |
| | | | C4.fxd | T | 0.031 | 0.22 | 0.0002 | 1.08 (1.01-1.17) |
| | | | C6.fxd | T | 0.030 | 0.23 | 0.0002 | 1.08 (1.01-1.17) |
| | | | C6.rnd | T | 0.15 | −0.056 | 8.8E−05 | 1.12 (0.96-1.31) |
| 1p36.31 | rs1876848 | 6,876,262 | FI | G | 0.13 | −0.018 | NA | 1.12 (0.97-1.30) |
| | | | CE | G | 3.0E−05 | 2.92 | NA | 1.25 (1.13-1.39) |
| | | | NL | G | 0.0011 | 1.56 | NA | 1.32 (1.12-1.55) |
| | | | AN | G | 0.36 | −0.18 | NA | 1.11 (0.89-1.38) |
| | | | DE | G | 0.0067 | 0.94 | NA | 1.27 (1.07-1.52) |
| | | | JP2 | A | 0.016 | 0.61 | NA | 1.17 (1.03-1.33) |
| | | | JP1 | NA | NA | NA | NA | NA |
| | | | D.fxd | G | 2.0E−05 | 3.10 | 0.1128 | 1.21 (1.11-1.32) |
| | | | JP.fxd | NA | NA | NA | NA | NA |
| | | | C4.fxd | G | 0.027 | 0.27 | 0.0002 | 1.08 (1.01-1.16) |
| | | | C6.fxd | G | 0.027 | 0.27 | 0.0002 | 1.08 (1.01-1.16) |
| | | | C6.rnd | G | 0.17 | −0.092 | 8.1E−05 | 1.12 (0.95-1.31) |
| 1p22.2 | rs1725390 | 91,031,160 | FI | A | 0.0014 | 1.48 | NA | 1.21 (1.08-1.36) |
| | | | CE | A | 0.0029 | 1.14 | NA | 1.13 (1.04-1.22) |
| | | | NL | A | 0.021 | 0.52 | NA | 1.16 (1.02-1.32) |
| | | | AN | A | 0.33 | −0.25 | NA | 1.08 (092-1.28) |
| | | | DE | A | 0.075 | 0.11 | NA | 1.13 (0.99-1.28) |
| | | | JP2 | A | 0.14 | −0.11 | NA | 1.10 (0.97-1.24) |
| | | | JP1 | A | 0.59 | −0.40 | NA | 1.04 (0.90-1.22) |
| | | | D.fxd | A | 2.0E−05 | 3.05 | 0.1011 | 1.16 (1.08-1.23) |
| | | | JP.fxd | A | 0.13 | −0.18 | 6.6E−05 | 1.08 (0.98-1.18) |
| | | | C4.fxd | A | 1.3E−05 | 3.16 | 0.1273 | 1.13 (1.07-1.19) |
| | | | C6.fxd | A | 1.3E−05 | 3.18 | 0.1303 | 1.13 (1.07-1.19) |
| | | | C6.rnd | A | 1.3E−05 | 3.18 | 0.1303 | 1.13 (1.07-1.19) |
| 1p22.2 | rs941031 | 91,067,737 | FI | T | 0.0045 | −1.06 | NA | 1.18 (1.05-1.33) |
| | | | CE | T | 0.0018 | 1.32 | NA | 1.14 (1.05-1.23) |
| | | | NL | T | 0.061 | 0.17 | NA | 1.13 (0.99-1.29) |
| | | | AN | T | 0.20 | −0.12 | NA | 1.11 (0.95-1.31) |
| | | | DE | T | 0.027 | 0.44 | NA | 1.16 (1.02-1.32) |
| | | | JP2 | T | 0.20 | −0.21 | NA | 1.08 (0.96-1.23) |
| | | | JP1 | C | 0.47 | −0.35 | NA | 1.06 (0.91-1.24) |
| | | | D.fxd | T | 2.9E−05 | 2.90 | 0.0737 | 1.15 (1.08-1.23) |
| | | | JP.fxd | T | 0.58 | −0.58 | 2.7E−05 | 1.03 (0.93-1.13) |
| | | | C4.fxd | T | 0.00017 | 2.13 | 0.0133 | 1.11 (1.05-1.17) |
| | | | C6.fxd | T | 0.00016 | 2.16 | 0.0142 | 1.11 (1.05-1.17) |
| | | | C6.rnd | T | 0.00037 | 1.85 | 0.0070 | 1.11 (1.05-1.17) |
| 1q21.3 | rs905938 | 153,258,013 | FI | T | 0.0067 | 0.94 | NA | 1.22 (1.06-1.41) |
| | | | CE | T | 0.00071 | 1.71 | NA | 1.17 (1.07-1.28) |
| | | | NL | T | 0.028 | 0.45 | NA | 1.18 (1.02-1.36) |
| | | | AN | T | 0.048 | 0.32 | NA | 1.21 (1.00-1.47) |
| | | | DE | T | 0.075 | 0.14 | NA | 1.14 (0.99-1.32) |
| | | | JP2 | T | 0.62 | −0.18 | NA | 1.08 (0.79-1.47) |
| | | | JP1 | T | 0.82 | −0.15 | NA | 1.05 (0.71-1.55) |
| | | | D.fxd | T | 1.7E−05 | 3.16 | 0.1252 | 1.18 (1.10-1.28) |
| | | | JP.fxd | T | 0.59 | −0.24 | 5.7E−05 | 1.07 (0.84-1.36) |
| | | | C4.fxd | T | 2.0E−05 | 3.07 | 0.1061 | 1.17 (1.09-1.26) |
| | | | C6.fxd | T | 1.9E−05 | 3.10 | 0.1126 | 1.17 (1.09-1.26) |
| | | | C6.rnd | T | 1.9E−05 | 3.10 | 0.1126 | 1.17 (1.09-1.26) |
| 2q33.1 | rs1429412 | 197,899,139 | FI | G | 0.00091 | 1.64 | NA | 1.22 (1.09-1.38) |
| | | | CE | G | 0.0088 | 0.73 | NA | 1.12 (1.03-1.21) |
| | | | NL | G | 0.00094 | 1.63 | NA | 1.25 (1.09-1.42) |
| | | | AN | G | 0.47 | −0.31 | NA | 1.07 (0.90-1.27) |
| | | | DE | G | 0.79 | −0.49 | NA | 1.02 (0.89-1.17) |
| | | | JP2 | A | 0.0067 | 0.92 | NA | 1.19 (1.05-1.34) |
| | | | JP1 | G | 0.27 | −0.20 | NA | 1.10 (0.93-1.29) |
| | | | D.fxd | G | 5.4E−05 | 2.66 | 0.0440 | 1.15 (1.07-1.23) |
| | | | JP.fxd | G | 0.14 | −0.17 | 6.7E−05 | 0.93 (0.84-1.02) |
| | | | C4.fxd | G | 0.014 | 0.43 | 0.0003 | 1.07 (1.01-1.13) |
| | | | C6.fxd | G | 0.014 | 0.42 | 0.0003 | 1.07 (1.01-1.13) |
| | | | C6.rnd | G | 0.25 | −0.28 | 5.2E−05 | 1.07 (0.95-1.21) |
| 2q33.1 | rs12472355 | 197,914,085 | FI | A | 0.00034 | 2.00 | NA | 1.24 (1.10-1.40) |
| | | | CE | A | 0.0085 | 0.74 | NA | 1.12 (1.03-1.22) |
| | | | NL | A | 0.0012 | 1.55 | NA | 1.24 (1.09-1.41) |
| | | | AN | A | 0.40 | −0.27 | NA | 1.08 (0.91-1.28) |
| | | | DE | A | 0.79 | −0.49 | NA | 1.02 (0.89-1.17) |
| | | | JP2 | C | 0.00051 | 1.85 | NA | 1.25 (1.10-1.42) |
| | | | JP1 | NA | NA | NA | NA | NA |
| | | | D.fxd | A | 2.7E−05 | 2.94 | 0.0801 | 1.16 (1.08-1.24) |
| | | | JP.fxd | NA | NA | NA | NA | NA |
| | | | C4.fxd | A | 0.042 | 0.041 | 0.0001 | 1.06 (1.00-1.13) |
| | | | C6.fxd | A | 0.043 | 0.033 | 0.0001 | 1.06 (1.00-1.13) |
| | | | C6.rnd | A | 0.46 | −0.33 | 4.7E−05 | 1.06 (0.90-1.25) |
| 2q33.1 | rs787997 | 197,924,516 | FI | A | 0.00028 | 2.08 | NA | 1.25 (1.11-1.41) |
| | | | CE | A | 0.0084 | 0.75 | NA | 1.12 (1.03-1.22) |
| | | | NL | A | 0.0010 | 1.59 | NA | 1.24 (1.09-1.42) |
| | | | AN | A | 0.38 | −0.26 | NA | 1.08 (0.91-1.29) |
| | | | DE | A | 0.84 | −0.49 | NA | 1.01 (0.89-1.16) |
| | | | JP2 | G | 7.6E−05 | 2.55 | NA | 1.29 (1.14-1.46) |
| | | | JP1 | NA | NA | NA | NA | NA |
| | | | D.fxd | A | 2.3E−05 | 3.00 | 0.0914 | 1.16 (1.08-1.24) |
| | | | JP.fxd | NA | NA | NA | NA | NA |
| | | | C4.fxd | A | 0.068 | −0.13 | 7.5E−05 | 1.06 (1.00-1.12) |
| | | | C6.fxd | A | 0.069 | −0.13 | 7.4E−05 | 1.06 (1.00-1.12) |
| | | | C6.rnd | A | 0.53 | −0.33 | 4.6E−05 | 1.06 (0.89-1.26) |
| 2q33.1 | rs787994 | 197,931,366 | FI | T | 0.00029 | 2.06 | NA | 1.25 (1.11-1.41) |
| | | | CE | T | 0.0076 | 0.79 | NA | 1.12 (1.03-1.22) |
| | | | NL | T | 0.00082 | 1.68 | NA | 1.25 (1.10-1.42) |
| | | | AN | T | 0.45 | −0.30 | NA | 1.07 (0.90-1.27) |
| | | | DE | T | 0.79 | −0.48 | NA | 1.02 (0.89-1.17) |
| | | | JP2 | C | 8.6E−05 | 2.51 | NA | 1.28 (1.13-1.46) |
| | | | JP1 | T | 0.070 | 0.19 | NA | 1.17 (0.99-1.38) |
| | | | D.fxd | T | 2.1E−05 | 3.04 | 0.0988 | 1.16 (1.08-1.24) |
| | | | JP.fxd | T | 0.041 | 0.24 | 0.0002 | 0.90 (0.81-1.00) |
| | | | C4.fxd | T | 0.018 | 0.33 | 0.0002 | 1.07 (1.01-1.13) |
| | | | C6.fxd | T | 0.019 | 0.32 | 0.0002 | 1.07 (1.01-1.13) |
| | | | C6.rnd | T | 0.34 | −0.29 | 5.1E−05 | 1.07 (0.93-1.24) |
| 4q31.23 | rs2163011 | 148,468,196 | FI | A | 0.015 | 0.65 | NA | 1.19 (1.03-1.38) |
| | | | CE | A | 0.048 | 0.16 | NA | 1.10 (1.00-1.21) |
| | | | NL | A | 0.0026 | 1.27 | NA | 1.25 (1.08-1.44) |
| | | | AN | G | 0.16 | −0.016 | NA | 1.15 (0.95-1.40) |
| | | | DE | A | 0.24 | −0.19 | NA | 1.10 (0.94-1.28) |
| | | | JP2 | A | 0.040 | 0.28 | NA | 1.13 (1.01-1.27) |
| | | | JP1 | A | 0.095 | 0.068 | NA | 1.13 (0.98-1.31) |
| | | | D.fxd | A | 0.0029 | 1.14 | 0.0014 | 1.13 (1.04-1.22) |
| | | | JP.fxd | A | 0.0082 | 0.79 | 0.0006 | 1.13 (1.03-1.24) |
| | | | C4.fxd | A | 6.8E−05 | 2.53 | 0.0331 | 1.13 (1.06-1.20) |
| | | | C6.fxd | A | 6.6E−05 | 2.54 | 0.0338 | 1.13 (1.06-1.20) |
| | | | C6.rnd | A | 0.0039 | 1.02 | 0.0011 | 1.12 (1.04-1.21) |
| 4q31.23 | rs6841581 | 148,620,640 | FI | G | 0.0040 | 1.10 | NA | 1.31 (1.09-1.58) |
| | | | CE | G | 0.00073 | 1.72 | NA | 1.23 (1.09-1.39) |
| | | | NL | G | 0.33 | −0.21 | NA | 1.10 (0.91-1.32) |
| | | | AN | G | 0.00077 | 1.44 | NA | 1.55 (1.20-2.01) |
| | | | DE | G | 0.050 | 0.31 | NA | 1.21 (1.00-1.47) |
| | | | JP2 | G | 0.0066 | 0.93 | NA | 1.19 (1.05-1.35) |
| | | | JP1 | G | 0.023 | 0.53 | NA | 1.21 (1.03-1.42) |
| | | | D.fxd | G | 1.1E−05 | 3.33 | 0.1750 | 1.25 (1.13-1.39) |
| | | | JP.fxd | G | 0.00042 | 1.93 | 0.0084 | 1.20 (1.08-1.32) |
| | | | C4.fxd | G | 2.2E−08 | 5.84 | 0.9857 | 1.22 (1.14-1.31) |
| | | | C6.fxd | G | 2.3E−08 | 5.82 | 0.9851 | 1.22 (1.14-1.31) |
| | | | C6.rnd | G | 2.3E−08 | 5.82 | 0.9850 | 1.22 (1.14-1.31) |
| 5q23.2 | rs570682 | 122,477,549 | FI | T | 0.00032 | 2.01 | NA | 1.27 (1.12-1.45) |
| | | | CE | T | 0.055 | 0.14 | NA | 1.10 (1.00-1.22) |
| | | | NL | T | 0.29 | −0.21 | NA | 1.09 (0.93-1.29) |
| | | | AN | T | 0.58 | −0.28 | NA | 1.06 (0.86-1.31) |
| | | | DE | T | 0.11 | 0.059 | NA | 1.14 (0.97-1.34) |
| | | | JP2 | T | 0.79 | −0.55 | NA | 1.02 (0.91-1.14) |
| | | | JP1 | NA | NA | NA | NA | NA |
| | | | D.fxd | T | 0.00021 | 2.16 | 0.0144 | 1.16 (1.07-1.26) |
| | | | JP.fxd | NA | NA | NA | NA | NA |
| | | | C4.fxd | T | 0.0014 | 1.36 | 0.0023 | 1.11 (1.04-1.19) |
| | | | C6.fxd | T | 0.0014 | 1.38 | 0.0024 | 1.11 (1.04-1.19) |
| | | | C6.rnd | T | 0.016 | 0.53 | 0.0003 | 1.12 (1.02-1.22) |
| 5q23.2 | rs2287696 | 122,488,231 | FI | A | 0.00036 | 1.97 | NA | 1.28 (1.12-1.46) |
| | | | CE | A | 0.0057 | 0.97 | NA | 1.17 (1.05-1.31) |
| | | | NL | A | 0.077 | 0.17 | NA | 1.17 (0.98-1.40) |
| | | | AN | A | 0.34 | −0.15 | NA | 1.12 (0.89-1.42) |
| | | | DE | A | 0.048 | 0.31 | NA | 1.20 (1.00-1.43) |
| | | | JP2 | A | 0.27 | −0.32 | NA | 1.07 (0.95-1.20) |
| | | | JP1 | NA | NA | NA | NA | NA |
| | | | D.fxd | A | 1.1E−05 | 3.33 | 0.1760 | 1.21 (1.11-1.32) |
| | | | JP.fxd | NA | NA | NA | NA | NA |
| | | | C4.fxd | A | 2.9E−05 | 2.92 | 0.0767 | 1.16 (1.08-1.24) |
| | | | C6.fxd | A | 2.7E−05 | 2.94 | 0.0805 | 1.16 (1.08-1.24) |
| | | | C6.rnd | A | 0.00026 | 2.08 | 0.0119 | 1.16 (1.07-1.26) |
| 5q23.2 | rs335206 | 122,532,465 | FI | C | 0.0059 | 0.97 | NA | 1.18 (1.05-1.34) |
| | | | CE | C | 0.0013 | 1.44 | NA | 1.14 (1.05-1.23) |
| | | | NL | C | 0.010 | 0.77 | NA | 1.18 (1.04-1.34) |
| | | | AN | C | 0.48 | −0.34 | NA | 1.06 (0.90-1.25) |
| | | | DE | C | 0.037 | 0.33 | NA | 1.14 (1.01-1.29) |
| | | | JP2 | C | 0.55 | −0.46 | NA | 1.04 (0.92-1.18) |
| | | | JP1 | NA | NA | NA | NA | NA |
| | | | D.fxd | C | 2.7E−05 | 2.92 | 0.0776 | 1.15 (1.08-1.23) |
| | | | JP.fxd | NA | NA | NA | NA | NA |
| | | | C4.fxd | C | 6.7E−05 | 2.54 | 0.0331 | 1.12 (1.06-1.19) |
| | | | C6.fxd | C | 6.5E−05 | 2.54 | 0.0338 | 1.12 (1.06-1.19) |
| | | | C6.rnd | C | 6.5E−05 | 2.54 | 0.0338 | 1.12 (1.06-1.19) |
| 8p23.2 | rs2045637 | 2,963,188 | FI | A | 9.2E−05 | 2.41 | NA | 1.37 (1.17-1.61) |
| | | | CE | A | 0.0080 | 0.85 | NA | 1.17 (1.04-1.32) |
| | | | NL | A | 0.12 | 0.054 | NA | 1.16 (0.96-1.40) |
| | | | AN | G | 0.93 | −0.27 | NA | 1.01 (0.78-1.31) |
| | | | DE | A | 0.0051 | 1.01 | NA | 1.31 (1.08-1.59) |
| | | | JP2 | G | 0.36 | −0.38 | NA | 1.06 (0.94-1.19) |
| | | | JP1 | NA | NA | NA | NA | NA |
| | | | D.fxd | A | 8.6E−06 | 3.43 | 0.2139 | 1.24 (1.13-1.37) |
| | | | JP.fxd | NA | NA | NA | NA | NA |
| | | | C4.fxd | A | 0.0036 | 1.03 | 0.0011 | 1.12 (1.04-1.20) |
| | | | C6.fxd | A | 0.0034 | 1.06 | 0.0011 | 1.12 (1.04-1.20) |
| | | | C6.rnd | A | 0.084 | 0.12 | 0.0001 | 1.14 (0.98-1.33) |
| 8q24.23 | rs1040247 | 139,602,549 | FI | A | 0.029 | 0.45 | NA | 1.19 (1.02-1.40) |
| | | | CE | A | 0.0012 | 1.53 | NA | 1.20 (1.07-1.33) |
| | | | NL | A | 0.17 | −0.056 | NA | 1.13 (0.95-1.34) |
| | | | AN | G | 0.82 | −0.29 | NA | 1.03 (0.81-1.30) |
| | | | DE | A | 4.1E−05 | 2.64 | NA | 1.44 (1.21-1.72) |
| | | | JP2 | A | 0.26 | −0.29 | NA | 1.07 (0.95-1.21) |
| | | | JP1 | G | 0.38 | −0.31 | NA | 1.07 (0.92-1.25) |
| | | | D.fxd | A | 9.7E−05 | 2.48 | 0.0293 | 1.20 (1.09-1.31) |
| | | | JP.fxd | A | 0.74 | −0.62 | 2.4E−05 | 1.02 (0.92-1.12) |
| | | | C4.fxd | A | 0.0022 | 1.19 | 0.0015 | 1.11 (1.04-1.18) |
| | | | C6.fxd | A | 0.0019 | 1.23 | 0.0017 | 1.11 (1.04-1.18) |
| | | | C6.rnd | A | 0.083 | 0.057 | 0.0001 | 1.11 (0.99-1.26) |
| 8q24.23 | rs1554349 | 139,604,536 | FI | A | 0.023 | 0.52 | NA | 1.20 (1.02-1.41) |
| | | | CE | A | 0.0012 | 1.53 | NA | 1.20 (1.07-1.33) |
| | | | NL | A | 0.16 | −0.043 | NA | 1.13 (0.95-1.34) |
| | | | AN | G | 0.83 | −0.29 | NA | 1.03 (0.81-1.30) |
| | | | DE | A | 5.0E−05 | 2.57 | NA | 1.43 (1.20-1.71) |
| | | | JP2 | A | 0.15 | −0.12 | NA | 1.09 (0.97-1.24) |
| | | | JP1 | G | 0.33 | −0.28 | NA | 1.08 (0.93-1.25) |
| | | | D.fxd | A | 7.9E−05 | 2.56 | 0.0349 | 1.20 (1.09-1.31) |
| | | | JP.fxd | A | 0.60 | −0.59 | 2.6E−05 | 1.03 (0.93-1.13) |
| | | | C4.fxd | A | 0.0012 | 1.41 | 0.0025 | 1.11 (1.04-1.19) |
| | | | C6.fxd | A | 0.0011 | 1.45 | 0.0028 | 1.11 (1.04-1.19) |
| | | | C6.rnd | A | 0.072 | 0.10 | 0.0001 | 1.12 (0.99-1.26) |
| 11q22.2 | rs2282652 | 101,602,525 | FI | C | 2.1E−05 | 3.03 | NA | 1.30 (1.15-1.47) |
| | | | CE | C | 0.024 | 0.36 | NA | 1.10 (1.01-1.19) |
| | | | NL | T | 0.72 | −0.49 | NA | 1.02 (0.90-1.17) |
| | | | AN | C | 0.0027 | 1.23 | NA | 1.30 (1.09-1.53) |
| | | | DE | C | 0.064 | 0.17 | NA | 1.14 (0.99-1.30) |
| | | | JP2 | C | 0.74 | −0.53 | NA | 1.02 (0.91-1.15) |
| | | | JP1 | NA | NA | NA | NA | NA |
| | | | D.fxd | C | 2.3E−05 | 3.01 | 0.0928 | 1.16 (1.08-1.24) |
| | | | JP.fxd | NA | NA | NA | NA | NA |
| | | | C4.fxd | C | 0.00013 | 2.27 | 0.0184 | 1.12 (1.06-1.19) |
| | | | C6.fxd | C | 0.00010 | 2.36 | 0.0224 | 1.12 (1.06-1.19) |
| | | | C6.rnd | C | 0.035 | 0.33 | 0.0002 | 1.13 (1.01-1.27) |
| 11q22.2 | rs2124216 | 101,644,113 | FI | A | 0.00045 | 1.90 | NA | 1.26 (1.11-1.43) |
| | | | CE | A | 0.0028 | 1.17 | NA | 1.14 (1.05-1.25) |
| | | | NL | A | 0.65 | −0.45 | NA | 1.03 (0.90-1.18) |
| | | | AN | A | 0.0027 | 1.21 | NA | 1.33 (1.10-1.60) |
| | | | DE | A | 0.025 | 0.49 | NA | 1.18 (1.02-1.36) |
| | | | JP2 | A | 0.20 | −0.24 | NA | 1.08 (0.96-1.21) |
| | | | JP1 | G | 0.65 | −0.43 | NA | 1.03 (0.89-1.20) |
| | | | D.fxd | A | 9.1E−06 | 3.39 | 0.1963 | 1.18 (1.10-1.27) |
| | | | JP.fxd | A | 0.47 | −0.55 | 2.8E−05 | 1.03 (0.94-1.13) |
| | | | C4.fxd | A | 9.1E−05 | 2.40 | 0.0248 | 1.12 (1.06-1.18) |
| | | | C6.fxd | A | 7.5E−05 | 2.48 | 0.0293 | 1.12 (1.06-1.19) |
| | | | C6.rnd | A | 0.011 | 0.69 | 0.0005 | 1.13 (1.03-1.23) |
| 12p13.31 | rs728342 | 5,577,633 | FI | G | 0.048 | 0.25 | NA | 1.14 (1.00-1.30) |
| | | | CE | G | 8.0E−05 | 2.53 | NA | 1.18 (1.09-1.28) |
| | | | NL | G | 0.044 | 0.28 | NA | 1.14 (1.00-1.30) |
| | | | AN | G | 0.090 | 0.12 | NA | 1.16 (0.98-1.37) |
| | | | DE | G | 0.0019 | 1.38 | NA | 1.23 (1.08-1.41) |
| | | | JP2 | G | 0.33 | −0.30 | NA | 1.07 (0.93-1.24) |
| | | | JP1 | G | 0.56 | −0.33 | NA | 1.06 (0.88-1.26) |
| | | | D.fxd | G | 1.2E−05 | 3.28 | 0.1601 | 1.17 (1.09-1.25) |
| | | | JP.fxd | G | 0.26 | −0.32 | 4.8E−05 | 1.07 (0.95-1.19) |
| | | | C4.fxd | G | 1.5E−05 | 3.13 | 0.1193 | 1.14 (1.07-1.21) |
| | | | C6.fxd | G | 1.3E−05 | 3.19 | 0.1335 | 1.14 (1.07-1.21) |
| | | | C6.rnd | G | 1.3E−05 | 3.19 | 0.1335 | 1.14 (1.07-1.21) |
| 12q22 | rs6538595 | 94,030,754 | FI | A | 0.017 | 0.61 | NA | 1.17 (1.03-1.34) |
| | | | CE | A | 0.00035 | 1.95 | NA | 1.16 (1.07-1.25) |
| | | | NL | A | 0.030 | 0.41 | NA | 1.15 (1.01-1.31) |
| | | | AN | A | 0.022 | 0.55 | NA | 1.22 (1.03-1.44) |
| | | | DE | A | 0.061 | 0.17 | NA | 1.13 (0.99-1.29) |
| | | | JP2 | A | 0.0051 | 1.01 | NA | 1.19 (1.05-1.34) |
| | | | JP1 | A | 0.13 | −0.012 | NA | 1.13 (0.97-1.31) |
| | | | D.fxd | A | 1.8E−05 | 3.11 | 0.1136 | 1.16 (1.08-1.24) |
| | | | JP.fxd | A | 0.0017 | 1.38 | 0.0024 | 1.16 (1.06-1.28) |
| | | | C4.fxd | A | 1.1E−07 | 5.15 | 0.9343 | 1.16 (1.10-1.23) |
| | | | C6.fxd | A | 9.6E−08 | 5.20 | 0.9404 | 1.16 (1.10-1.23) |
| | | | C6.rnd | A | 9.6E−08 | 5.20 | 0.9404 | 1.16 (1.10-1.23) |
| 12q22 | rs6419373 | 94,091,596 | FI | C | 0.042 | 0.30 | NA | 1.14 (1.00-1.30) |
| | | | CE | C | 0.0025 | 1.19 | NA | 1.13 (1.04-1.23) |
| | | | NL | C | 0.13 | −0.081 | NA | 1.10 (0.97-1.25) |
| | | | AN | C | 0.091 | 0.12 | NA | 1.16 (0.98-1.38) |
| | | | DE | C | 0.033 | 0.37 | NA | 1.15 (1.01-1.31) |
| | | | JP2 | C | 0.0066 | 0.92 | NA | 1.17 (1.05-1.32) |
| | | | JP1 | C | 0.059 | 0.21 | NA | 1.15 (0.99-1.33) |
| | | | D.fxd | C | 0.00027 | 2.01 | 0.0102 | 1.14 (1.06-1.22) |
| | | | JP.fxd | C | 0.00098 | 1.59 | 0.0039 | 1.17 (1.06-1.28) |
| | | | C4.fxd | C | 1.0E−06 | 4.22 | 0.6246 | 1.15 (1.09-1.21) |
| | | | C6.fxd | C | 9.2E−07 | 4.26 | 0.6477 | 1.15 (1.09-1.21) |
| | | | C6.rnd | C | 9.2E−07 | 4.26 | 0.6477 | 1.15 (1.09-1.21) |
| 19q13.12 | rs1688005 | 40,340,205 | FI | G | 0.038 | 0.32 | NA | 1.14 (1.01-1.29) |
| | | | CE | G | 0.00013 | 2.34 | NA | 1.19 (1.09-1.30) |
| | | | NL | G | 0.020 | 0.55 | NA | 1.17 (1.02-1.34) |
| | | | AN | G | 0.010 | 0.80 | NA | 1.28 (1.06-1.54) |
| | | | DE | G | 0.057 | 0.23 | NA | 1.15 (1.00-1.34) |
| | | | JP2 | T | 0.16 | −0.069 | NA | 1.12 (0.96-1.31) |
| | | | JP1 | NA | NA | NA | NA | NA |
| | | | D.fxd | G | 1.6E−05 | 3.15 | 0.1244 | 1.17 (1.09-1.26) |
| | | | JP.fxd | NA | NA | NA | NA | NA |
| | | | C4.fxd | G | 0.00080 | 1.58 | 0.0038 | 1.12 (1.05-1.19) |
| | | | C6.fxd | G | 0.00072 | 1.62 | 0.0042 | 1.12 (1.05-1.19) |
| | | | C6.rnd | G | 0.046 | 0.22 | 0.0002 | 1.12 (1.00-1.25) |
| 20p12.1 | rs1132274 | 17,544,155 | FI | A | 0.010 | 0.79 | NA | 1.23 (1.05-1.45) |
| | | | CE | A | 0.00044 | 1.90 | NA | 1.21 (1.09-1.34) |
| | | | NL | A | 0.0033 | 1.17 | NA | 1.27 (1.08-1.50) |
| | | | AN | A | 0.94 | −0.30 | NA | 1.01 (0.80-1.28) |
| | | | DE | A | 0.0094 | 0.83 | NA | 1.26 (1.06-1.50) |
| | | | JP2 | A | 0.012 | 0.69 | NA | 1.16 (1.03-1.30) |
| | | | JP1 | NA | NA | NA | NA | NA |
| | | | D.fxd | A | 1.5E−05 | 3.22 | 0.1435 | 1.22 (1.11-1.33) |
| | | | JP.fxd | NA | NA | NA | NA | NA |
| | | | C4.fxd | A | 6.9E−07 | 4.43 | 0.7279 | 1.20 (1.11-1.28) |
| | | | C6.fxd | A | 6.4E−07 | 4.46 | 0.7412 | 1.20 (1.11-1.28) |
| | | | C6.rnd | A | 6.5E−07 | 4.45 | 0.7379 | 1.20 (1.11-1.28) |
| 22q12.1 | rs133885 | 24,489,289 | FI | G | 0.22 | −0.26 | NA | 1.08 (0.96-1.21) |
| | | | CE | G | 1.0E−05 | 3.33 | NA | 1.20 (1.10-1.30) |
| | | | NL | G | 0.00088 | 1.65 | NA | 1.24 (1.09-1.41) |
| | | | AN | G | 0.26 | −0.19 | NA | 1.10 (0.93-1.29) |
| | | | DE | G | 0.0034 | 1.17 | NA | 1.21 (1.07-1.38) |
| | | | JP2 | G | 0.67 | −0.42 | NA | 1.03 (0.89-1.20) |
| | | | JP1 | NA | NA | NA | NA | NA |
| | | | D.fxd | G | 1.6E−05 | 3.15 | 0.1230 | 1.16 (1.08-1.24) |
| | | | JP.fxd | NA | NA | NA | NA | NA |
| | | | C4.fxd | G | 3.6E−05 | 2.79 | 0.0584 | 1.14 (1.07-1.21) |
| | | | C6.fxd | G | 3.4E−05 | 2.82 | 0.0614 | 1.14 (1.07-1.21) |
| | | | C6.rnd | G | 0.00048 | 1.80 | 0.0063 | 1.14 (1.06-1.22) |

RA, risk allele;
CE, unstratified cohort consisting of NL, AN and DE;
C4, the combined result of FI, CE, JP2 and JP1;
C6, the combined result of FI, NL, AN, DE, JP2 and JP1;
fxd and rnd indicate the fixed- and random-effects model, respectively.
Note that the per-allele odds ratio (OR) was calculated for the designated risk allele.

TABLE 5
Results of two-locus interaction analysis
for SNPs associated with IA.

| | | | Interaction | Interaction OR |
|---|---|---|---|---|
| SNP1 | SNP2 | Cohort | P-value | (95% CI) |
| rs12413409 | rs6841581 | CE | 0.051 | 0.71 (0.50-1.00) |
| rs12413409 | rs6841581 | FI | 0.12 | 0.64 (0.36-1.12) |
| rs12413409 | rs6841581 | JP | 0.65 | 1.04 (0.88-1.23) |
| rs6538595 | rs6841581 | CE | 0.5 | 1.06 (0.90-1.26) |
| rs6538595 | rs6841581 | FI | 0.85 | 1.03 (0.78-1.36) |
| rs6538595 | rs6841581 | JP | 0.4 | 1.07 (0.92-1.24) |
| rs9315204 | rs6841581 | CE | 0.36 | 1.09 (0.90-1.32) |
| rs9315204 | rs6841581 | FI | 0.044 | 1.32 (1.01-1.73) |
| rs9315204 | rs6841581 | JP | 0.6 | 1.04 (0.89-1.23) |
| rs11661542 | rs6841581 | CE | 0.44 | 0.94 (0.79-1.11) |
| rs1661542 | rs6841581 | FI | 0.37 | 0.89 (0.69-1.15) |
| rs11661542 | rs6841581 | JP | 0.84 | 1.02 (0.88-1.18) |
| rs1132274 | rs6841581 | CE | 0.2 | 1.16 (0.92-1.46) |
| rs1132274 | rs6841581 | FI | 0.3 | 1.21 (0.84-1.74) |
| rs1132274 | rs6841581 | JP | 0.9 | 1.01 (0.87-1.17) |
| rs6841581 | rs10958409 | CE | 0.24 | 0.88 (0.72-1.09) |
| rs6841581 | rs10958409 | FI | 0.48 | 0.9 (0.66-1.22) |
| rs6841581 | rs10958409 | JP | 0.78 | 1.02 (0.87-1.20) |
| rs12413409 | rs10958409 | CE | 0.089 | 1.27 (0.96-1.68) |
| rs12413409 | rs10958409 | FI | 0.27 | 0.81 (0.55-1.18) |
| rs12413409 | rs10958409 | JP | 0.51 | 0.94 (0.80-1.12) |
| rs6538595 | rs10958409 | CE | 0.013 | 0.83 (0.72-0.96) |
| rs6538595 | rs10958409 | FI | 0.22 | 1.15 (0.92-1.44) |
| rs6538595 | rs10958409 | JP | 0.17 | 1.11 (0.96-1.29) |
| rs9315204 | rs10958409 | CE | 0.23 | 1.11 (0.94-1.31) |
| rs9315204 | rs10958409 | FI | 0.23 | 0.88 (0.71-1.08) |
| rs9315204 | rs10958409 | JP | 0.57 | 0.95 (0.80-1.13) |
| rs11661542 | rs10958409 | CE | 0.56 | 1.04 (0.91-1.20) |
| rs11661542 | rs10958409 | FI | 0.68 | 1.04 (0.85-1.28) |
| rs11661542 | rs10958409 | JP | 0.94 | 0.99 (0.85-1.16) |
| rs1132274 | rs10958409 | CE | 0.12 | 0.85 (0.70-1.04) |
| rs1132274 | rs10958409 | FI | 0.25 | 0.85 (0.65-1.12) |
| rs1132274 | rs10958409 | JP | 0.59 | 0.96 (0.83-1.11) |
| rs6841581 | rs9298506 | CE | 0.55 | 0.93 (0.75-1.17) |
| rs6841581 | rs9298506 | FI | 0.48 | 0.89 (0.65-1.23) |
| rs6841581 | rs9298506 | JP | 0.11 | 0.87 (0.72-1.04) |
| rs10958409 | rs9298506 | CE | 0.17 | 0.88 (0.72-1.06) |
| rs10958409 | rs9298506 | FI | 0.93 | 1.01 (0.79-1.30) |
| rs10958409 | rs9298506 | JP | 0.23 | 0.89 (0.73-1.08) |
| rs12413409 | rs9298506 | CE | 0.57 | 0.92 (0.68-1.23) |
| rs12413409 | rs9298506 | FI | 0.57 | 0.89 (0.60-1.32) |
| rs12413409 | rs9298506 | JP | 0.76 | 1.03 (0.85-1.25) |
| rs6538595 | rs9298506 | CE | 0.34 | 0.93 (0.80-1.08) |
| rs6538595 | rs9298506 | FI | 0.24 | 0.88 (0.70-1.09) |
| rs6538595 | rs9298506 | JP | 0.11 | 0.87 (0.73-1.03) |
| rs9315204 | rs9298506 | CE | 0.97 | 1 (0.84-1.19) |
| rs9315204 | rs9298506 | FI | 0.56 | 1.06 (0.86-1.31) |
| rs9315204 | rs9298506 | JP | 0.48 | 1.07 (0.89-1.29) |
| rs11661542 | rs9298506 | CE | 0.52 | 0.95 (0.82-1.10) |
| rs11661542 | rs9298506 | FI | 0.068 | 1.2 (0.99-1.47) |
| rs11661542 | rs9298506 | JP | 0.32 | 0.92 (0.78-1.08) |
| rs1132274 | rs9298506 | CE | 0.092 | 0.85 (0.70-1.03) |
| rs1132274 | rs9298506 | FI | 0.59 | 1.08 (0.81-1.45) |
| rs1132274 | rs9298506 | JP | 0.19 | 1.12 (0.95-1.31) |
| rs6841581 | rs1333040 | CE | 0.043 | 1.19 (1.01-1.42) |
| rs6841581 | rs1333040 | FI | 0.47 | 0.91 (0.69-1.18) |
| rs6841581 | rs1333040 | JP | 0.26 | 1.09 (0.94-1.28) |
| rs10958409 | rs1333040 | CE | 0.51 | 1.05 (0.91-1.22) |
| rs10958409 | rs1333040 | FI | 0.43 | 0.92 (0.75-1.13) |
| rs10958409 | rs1333040 | JP | 0.42 | 1.07 (0.91-1.25) |
| rs9298506 | rs1333040 | CE | 0.3 | 1.09 (0.93-1.27) |
| rs9298506 | rs1333040 | FI | 0.4 | 0.91 (0.74-1.13) |
| rs9298506 | rs1333040 | JP | 1 | 1 (0.84-1.19) |
| rs12413409 | rs1333040 | CE | 0.73 | 1.04 (0.83-1.30) |
| rs12413409 | rs1333040 | FI | 0.19 | 0.81 (0.59-1.11) |
| rs12413409 | rs1333040 | JP | 0.93 | 0.99 (0.84-1.17) |
| rs6538595 | rs1333040 | CE | 0.41 | 0.95 (0.85-1.07) |
| rs6538595 | rs1333040 | FI | 0.81 | 0.98 (0.81-1.17) |
| rs6538595 | rs1333040 | JP | 0.58 | 1.04 (0.90-1.21) |
| rs9315204 | rs1333040 | CE | 0.85 | 0.99 (0.86-1.13) |
| rs9315204 | rs1333040 | FI | 0.26 | 0.91 (0.77-1.07) |
| rs9315204 | rs1333040 | JP | 0.66 | 0.96 (0.82-1.13) |
| rs11661542 | rs1333040 | CE | 0.31 | 1.06 (0.95-1.19) |
| rs11661542 | rs1333040 | FI | 0.076 | 1.16 (0.98-1.37) |
| rs11661542 | rs1333040 | JP | 0.66 | 1.03 (0.89-1.20) |
| rs1132274 | rs1333040 | CE | 0.16 | 0.89 (0.77-1.05) |
| rs1132274 | rs1333040 | FI | 0.056 | 0.8 (0.63-1.01) |
| rs1132274 | rs1333040 | JP | 0.34 | 0.93 (0.81-1.08) |
| rs12413409 | rs6538595 | CE | 0.82 | 1.03 (0.83-1.27) |
| rs12413409 | rs6538595 | FI | 0.44 | 0.88 (0.63-1.22) |
| rs12413409 | rs6538595 | JP | 0.36 | 1.08 (0.92-1.26) |
| rs12413409 | rs9315204 | CE | 0.89 | 0.98 (0.77-1.25) |
| rs12413409 | rs9315204 | FI | 0.41 | 1.15 (0.83-1.59) |
| rs12413409 | rs9315204 | JP | 0.7 | 1.03 (0.87-1.24) |
| rs6538595 | rs9315204 | CE | 0.75 | 0.98 (0.86-1.12) |
| rs6538595 | rs9315204 | FI | 0.71 | 0.97 (0.80-1.16) |
| rs6538595 | rs9315204 | JP | 0.67 | 0.97 (0.82-1.13) |
| rs12413409 | rs11661542 | CE | 0.97 | 1 (0.81-1.24) |
| rs12413409 | rs11661542 | FI | 0.79 | 1.04 (0.76-1.43) |
| rs12413409 | rs11661542 | JP | 0.82 | 1.02 (0.87-1.19) |
| rs6538595 | rs11661542 | CE | 0.52 | 0.97 (0.87-1.07) |
| rs6538595 | rs11661542 | FI | 0.46 | 0.93 (0.78-1.12) |
| rs6538595 | rs11661542 | JP | 0.34 | 0.93 (0.81-1.07) |
| rs9315204 | rs11661542 | CE | 0.24 | 1.08 (0.95-1.23) |
| rs9315204 | rs11661542 | FI | 0.27 | 1.1 (0.93-1.30) |
| rs9315204 | rs11661542 | JP | 0.44 | 0.94 (0.80-1.10) |
| rs12413409 | rs1132274 | CE | 0.14 | 1.26 (0.93-1.73) |
| rs12413409 | rs1132274 | FI | 0.3 | 0.8 (0.52-1.22) |
| rs12413409 | rs1132274 | JP | 0.96 | 1 (0.86-1.17) |
| rs6538595 | rs1132274 | CE | 0.0068 | 0.81 (0.70-0.94) |
| rs6538595 | rs1132274 | FI | 0.46 | 1.1 (0.86-1.40) |
| rs6538595 | rs1132274 | JP | 0.37 | 1.06 (0.93-1.22) |
| rs9315204 | rs1132274 | CE | 0.18 | 1.12 (0.95-1.34) |
| rs9315204 | rs1132274 | FI | 0.57 | 1.07 (0.84-1.36) |
| rs9315204 | rs1132274 | JP | 0.63 | 0.96 (0.83-1.12) |
| rs11661542 | rs1132274 | CE | 0.16 | 0.9 (0.78-1.04) |
| rs11661542 | rs1132274 | FI | 0.27 | 1.13 (0.91-1.41) |
| rs11661542 | rs1132274 | JP | 0.47 | 0.95 (0.83-1.09) |

TABLE 6
Risk estimates using 7 IA risk loci.

| | | | | | | | | | Top 5% | Top 1% | | Fraction of |
|---|---|---|---|---|---|---|---|---|---|---|---|---|
| | Bottom | | Bottom | | | | | | vs. | vs. | Sibling | Sibling |
| | 1% | | 5% | | Top 5% | | Top 1% | | Bottom | Bottom | Recurrence | Recurrence |
| Cohort | n | OR | n | OR | n | OR | n | OR | 5% | 1% | Risk | Risk |
| FI | 47 | 0.33 | 220 | 0.42 | 220 | 2.25 | 47 | 2.80 | 5.37 | 8.39 | 1.089 | 0.061 |
| CE | 82 | 0.37 | 407 | 0.47 | 407 | 1.96 | 82 | 2.31 | 4.18 | 6.27 | 1.064 | 0.044 |
| JP | 15 | 0.40 | 73 | 0.49 | 76 | 1.98 | 21 | 2.28 | 4.01 | 5.74 | 1.059 | 0.041 |

n, the number of control subjects in the specific tail of the distribution of risk score.
OR, the odds ratio with respect to the mean risk score in the control sample.
Top x % vs. Bottom x % (x = 5, 1), the odds ratio compared between subjects within top and bottom x %.
Fraction of sibling recurrence risk was estimated using the sibling recurrence risk of IA of 4.

### Association of IA

Given the very poor prognosis after cerebral hemorrhage, diagnosis of aneurysm prior to rupture is paramount, so that intervention, such as surgical or endovascular repair, can be used diminish or prevent morbidity and mortality. While hypertension, smoking and positive family history increase the likelihood of intracranial aneurysm formation and/or rupture, these are of limited clinical value in identifying at-risk individuals, Siblings of intracranial aneurysm probands are at an estimated 4-fold increased risk of hemorrhagic stroke due to intracranial aneurysm compared to the general population, suggesting a significant genetic component to risk; the pattern of recurrence is generally consistent with multifactorial determination.

Association of intracranial aneurysms with the following genomic regions and genes were found:

TABLE 7A

| | | | | Per-allele OR | |
|---|---|---|---|---|---|
| Chr | SNP | Position | P-value | (95% CI) | Gene(s) |
| 4 | rs6841581 | 148,620,640 | 3.8E−08 | 1.22 (1.14-1.31) | EDNRA |
| 8 | rs9298506 | 55,600,077 | 1.3E−12 | 1.28 (1.20-1.38) | SOX17 |
| 9 | rs1333040 | 22,073,404 | 1.5E−22 | 1.32 (1.25-1.39) | CDKN2A, |
| | | | | | CDKN2B |
| 10 | rs12413409 | 104,709,086 | 1.2E−09 | 1.29 (1.19-1.40) | CNNM2 |
| 12 | rs6538595 | 94,030,754 | 1.4E−08 | 1.17 (1.11-1.24) | FGD6, |
| | | | | | VEZT |
| 13 | rs9315204 | 32,591,837 | 2.5E−09 | 1.20 (1.13-1.28) | KL, |
| | | | | | STARD13 |
| 18 | rs11661542 | 18,477,693 | 1.1E−12 | 1.22 (1.15-1.28) | RBBP8 |
| 20 | rs1132274 | 17,544,155 | 6.9E−07 | 1.20 (1.11-1.28) | DSTN, |
| | | | | | RRBP1 |

TABLE 7B

| Reference | | SEQ ID |
|---|---|---|
| SNP ID* | Sequence | NO. |
| rs6841581 | CCACAAGTCTTTGTGGAGAGACGCAC[A/C/G/T]TGGAGAAAA | 1 |
| | ACTAACACTCAACACC | |
| rs9298506 | TGTTTTCCTCAGACAGGACCTTGTCA[A/G]CGCTTTCAAATAT | 2 |
| | GTAGGCTGTTT | |
| rs1333040 | AGACAGGAGGGTCAGAGGTAAGAATG[C/T]TACCGCTGGGAC | 3 |
| | AGAGAGGAAGGTA | |
| rs12413409 | TCAAGCTTTTTGTAGAAGAGTAAATG[A/G]TGTTGTGCTTGTA | 4 |
| | ACCCAGGAAGCT | |
| rs6538595 | AGTACTACTAGGGCTGAGGGAAGGCT[A/G]ATATCCTATTGTT | 5 |
| | AGACAGTGCCAG | |
| rs9315204 | ATTCTTAGTAATATTTCTTTGCATTC[C/T]AAGGTTTATCGCTC | 6 |
| | TAATCCCACAC | |
| rs11661542 | ACCCACAACAATTAGTTCCCTGAGAT[A/C]TTTTCACAAAGTC | 7 |
| | CAGGACCCAGTA | |
| rs1132274 | CGGCAAGGCTCAGACCTCGGCATGTC[G/T]GTTACAAGAAGA | 8 |
| | ATTGGAGAAGCTC | |

(*www.ncbi.nlm.nih.gov/snp)

The significant associations of these loci with intracranial aneurysm susceptibility have implications for pre-morbid diagnosis of individuals with IA. These common variants are utilized in the development of cost-effective and easily applicable genetic screening tests. In particular, the genotype of the patients at a particular locus, such as EDNRA, is used not only for risk prediction but also for treatment guidance, including but not limited to the decision whether a patient is likely to respond to a specific medication.

Identification of these genes also identifies new therapeutic targets.

Therapies aimed at these genes, their protein products and on other genes that interact with them define new approaches to treat aneurysms. In particular, for example, the known endothelin receptor antagonists such as sitaxentan, ambrisentan, atrasentan, BQ-123, bosentan and tezosentan are potential therapeutic modulators for IA,

The genes identified are involved in the generation, survival and maintenance of vascular and endothelial stem/progenitor cells. Besides providing insight in to the previously unknown biology of intracranial aneurysms, identification of the roles that these cells have permits the development of pharmacologic and stem-cell based treatment of IAs.

### Example 2

### Genome-Wide Association Study of Intracranial Aneurysm Identifies Three New Risk Loci

Saccular intracranial aneurysms are balloon-like dilations of the intracranial arterial wall; their hemorrhage commonly results in severe neurologic impairment and death. A second genome-wide association study is reported herein, with discovery and replication cohorts from Europe and Japan comprising 5,891 cases and 14,181 controls with ˜832,000 genotyped and imputed SNPs across discovery cohorts. Three new loci showing strong evidence for association with intracranial aneurysms in the combined dataset were identified, including intervals near RBBP8 on 18q11.2 (odds ratio (OR)=1.22, P=1.1×10⁻¹²), STARD13-KL on 13q13.1 (OR=1.20, P=2.5×10⁻⁹) and a gene-rich region on 10q24.32 (OR=1.29, P=1.2×10⁻⁹). Prior associations near SOX17 (8q11.23-q12.1; OR=1.28, P=1.3×10⁻¹²) and CDKN2A-CDKN2B (9p21.3; OR=1.31, P=1.5×10⁻²²) were also confirmed. It is noteworthy that several putative risk genes play a role in cell-cycle progression, potentially affecting the proliferation and senescence of progenitor-cell populations that are responsible for vascular formation and repair.

The materials and methods employed in these experiments are now described

Study Subjects

Phenotype Description:

In all cases, diagnosis of an intracranial aneurysm (IA) was made either with computerized tomography angiogram, magnetic resonance angiogram or cerebral digital subtraction angiogram and confirmed at surgery, when applicable. Rupture of an aneurysm was defined by identification of acute subarachnoid or intracranial hemorrhage (through computerized tomography or magnetic resonance imaging) from a proven aneurysm. Subjects with subarachnoid hemorrhage without saccular IA, non-saccular intracranial aneurysm (such as fusiform aneurysms) and those with known genetic syndromes that are believed to predispose to intracranial aneurysm (e.g. polycystic kidney disease and Ehlers-Danlos syndrome Type IV) were excluded from the study.

Discovery Cohort:

Genome-wide case-control data consisted of 11 cohorts (Table 11 and Table 12). Four of these were reported in a prior intracranial aneurysm genome-wide association study (GWAS) (Bilguvar et al., 2008, Nat Genet, 40: 1472-1477): Finnish case-control cohort (FI case-control, 912 cases and 740 controls), Dutch case (NL cases, n=786) and 2 control cohorts (Utrecht neurologically normal subjects (van Es et al., 2007, Lancet Neurol, 6: 869-877) (n=450) and the Rotterdam study (Hofman et al., 2007, Eur J Epidemiol, 22: 819-829) (n=5,974)), 5 previously-genotyped control cohorts: Finland Health 2000 (Keskitalo et al., 2009, Hum Mol Genet, 18, 4007-4012) (n=2,138), Northern Finnish Birth Cohort 1966 (Sabatti et al., 2009, Nat Genet, 41: 35-46) (n=5,302), KORA-gen (Wichmann et al., 2005, Gesundheitswesen, 67 Suppl 1, S26-30) (n=840), PopGen (Krawczak et al., 2006, Community Genet, 9: 55-61) (n=493) and Illumina's iControlDB (n=3,182, www.illumina.com/science/icontroldb.ilmn), and 2 newly ascertained case series: the Germany cohort (DE cases, n=975) which consisted of cases ascertained from Bonn, Dresden, Essen, Frankfurt and Tubingen, and the @neurIST case series (n=641) collected from Germany, Great Britain, Hungary, The Netherlands, Switzerland and Spain.

Replication Cohorts:

Two independent Japanese case-control cohorts were analyzed to confirm association signals detected in the discovery cohort. JP1: This cohort was described previously (Bilguvar et al., 2008, Nat Genet, 40, 1472-1477). Since the description of this cohort, additional cases (n=334) and controls (n=85) were ascertained. The sample size of this cohort is 829 cases and 761 controls. JP2: Case subjects (n=2,282) were obtained from the BioBank Japan at the Institute of Medical Science, the University of Tokyo (Nakamura, 2007, Clin Adv Hematol Oncol, 5: 696-697) while control subjects (n=905) from volunteers in the Osaka-Midosuji Rotary Club, Osaka, Japan (Kamatani et al., 2009, Nat Genet, 41: 35-46).

Genotyping

Whole-genome genotyping for the discovery cohort was performed on the Illumina platform according to the manufacturer's protocol (Illumina). Beadchips used for individual cohorts are presented in Table 11 and Table 12, Replication genotyping in the JP1 cohort was performed using either Taqman (Applied Biosystems) or MassARRAY (Sequenom) assays. For the JP2 cohort, genotyping for cases was performed using the multiplex PCR-based Invader assay (Third Wave Technologies Inc.); genotyping for controls was performed on an Illumina platform as described previously (Kamatani et al., 2009, Nat Genet, 41: 591-595).

Ethics

The study protocol was approved by the Yale Human Investigation Committee (HIC protocol #7680). Institutional review board approval for genetic studies, along with written consent from all study participants, was obtained at all participating institutions.

Data Storage and Analysis Tools

PLINK (Purcell et al., 2007, Am J Hum Genet, 81: 559-575) v1.06 and R statistical environment v2.9.0 (in particular, the snpMatrix package (Clayton et al., 2007, Hum Hered, 64: 45-51) was used for storage of genotype data and data analysis.

Preprocessing

Prior to the analysis of genotyping data, SNPs that were located either on mitochondrial DNA or sex chromosomes, SNPs with A/T or C/G alleles, those for which all subjects were assigned as ‘no call’, and those that were assayed on Hap300v1 or 550v1 but were dropped from newer versions were excluded.

Sample Quality Control

Subjects in the discovery cohort who did not conform to the study design were excluded on the basis of genotyping and information quality, cryptic relatedness and population outliers. The sample exclusion steps are summarized in Table 11. This filtering process resulted in the inclusion of 835 cases and 6,529 controls in the Finnish cohort and 2,000 cases and 8,722 controls in the rest of the combined European cohort.

Imputation

Imputation analysis with the HapMap phase II CEU reference panel (release 24) was performed using the IMPUTE v1 software (Marchini et al., 2007, Nat Genet, 39: 906-913). The analysis was performed separately for the Finnish and European cohorts. Posterior probabilities of three possible genotypes were converted to fractional allele dosage scores (between 0 and 2) and used these scores for association tests in order to take into account the imputation uncertainty (de Bakker et al., 2008, Hum Mol Genet, 17: R122-R128). For the quality assessment of imputed SNPs, the posterior probabilities were also converted to the most likely genotypes with the threshold at 0.9.

Case-Control Matching

Population stratification and independent genotyping of cases and controls are major causes of confounding in GWAS (Clayton et al., 2005, Nat Genet, 37: 1243-1246). Because the present study consisted of multiple independently ascertained cohorts that were genotyped separately, a stringent analysis was performed to control for these biases by inferring the genetic ancestries of subjects (Patterson et al., 2006, PLoS Genet, 2: e190; Price et al., 2006, Nat Genet, 38: 904-909). The Laplacian eigenmaps (Belkin et al., 2003, Neural Comput, 15: 1373-1396) were used to infer population structure. Following the determination of the number of dimensions (K+1) using the threshold given elsewhere (Lee et al., 2009, Genet Epidemiol, 34: 51-59), the K-dimensional nontrivial generalized eigenvectors (von Luxburg, 2007, Stat Comput, 17, 395-416) were used to calculate the Euclidean distance between any two subjects.

In the course of this analysis, ‘isolated’ subjects who were identified by using the nearest-neighbor distance distributions in any of the two-dimensional sections were excluded. After excluding these subjects, 13 dimensions in the Finnish cohort and 5 dimensions in the European cohort were observed. The larger dimensions observed in the Finnish sample could be attributable to the presence of many isolated populations in Finland.

Before matching, data was stratified into males and females because female gender is a known risk factor of intracranial aneurysm (Rinkel et al., 1998, Stroke, 29: 251-256; Iwamoto et al., 1999, Stroke, 30: 1390-1395). The maximum distance between cases and controls to match was set to be less than 0.028 in the Finnish cohort and 0.009 in the European cohort. These values were determined by examining the distribution of the nearest-neighbor distances in K dimensions (data not shown). Cases and controls were matched using the fullmatch function in the R-package optmatch (Rosenbaum, 1991, J.R. Statist Soc B, 53: 597-610; Hansen et al., 2006, J Comput Graph Statist, 15: 609-627).

SNP Quality Control

For both genotyped and imputed SNPs in the discovery cohort, quality-control filters were applied to individual cohorts and to cases and controls separately on the basis of the missing rate, MAF and the P value of the exact test of Hardy-Weinberg equilibrium (Wigginton et al., 2005, Am J Hum Genet, 76: 887-893). For imputed SNPs, imputation quality was also assessed using the average posterior probability, MAF and allelic R² metric (Browning et al., 2009, Am J Hum Genet, 84: 210-223). Finally, differential missingness between cases and controls was assessed (Table 12).

Any genotyped SNP that passed the quality-control filters both in the European and Finnish cohorts was referred to as a ‘genotyped SNP’. Any one for which the quality control-passed imputation data either in one or both of the cohorts was used was classified as an ‘imputed SNP’.

For genotyping data of the replication cohorts, SNPs were excluded if any of the following three conditions were met in either cases or controls: (i) missing rate>0.05; (ii) P value of the exact test of Hardy-Weinberg equilibrium<0.001; or (iii) MAF<0.01.

Statistical Analysis

Cohort-Wise Association Analysis.

Association between each quality control-passed SNP and intracranial aneurysm was tested for using conditional and unconditional logistic regression for the discovery and replication cohorts, respectively (Breslow et al., 1980, Statistical methods in cancer research. Volume I—the analysis of case-control studies. IARC Sci. Publ, 5-338). For the discovery cohort, the matched strata were used to correct for potential confounding due to population stratification and gender. For the replication cohorts, gender was adjusted for. The log-additive effect of allele dosage on disease risk was assumed. P values from the score test (two-sided) were obtained and the logarithm of per-allele ORs with standard errors were estimated by maximizing the conditional or unconditional likelihood. Both the test statistic and the standard error of the log of the OR were corrected using genomic control (Devlin et al., 1999, Biometrics, 55: 997-1004). The association analysis was performed for the Finnish and European cohorts, as well as subcohorts of the European group that consisted of NL cases, DE cases or @neurIST cases and their matched controls (Table 8 and Table 13). The following R functions were used to perform the association analysis: clogit, glmn and snp.rhs.tests (Clayton et al., 2007, Hum Hered, 64: 45-51).

Meta-Analysis.

The cohort-wise per-allele ORs in the Finnish and European cohorts were combined using a fixed-effects model of meta-analysis for 831,534 quality control-passed SNPs to obtain the discovery results. For SNPs analyzed both in the discovery and replication cohorts, JP1 and JP2 were combined to obtain replication results and all four cohorts were combined to obtain combined results. Primary analysis was based on the fixed-effects model (de Bakker et al., 2008, Hum Mot Genet, 17: R122-R128). To assess the heterogeneity of the effect size between cohorts, the European cohort were first divided into three groups as described above, aiming to analyze the data without averaging effect sizes over the combined European cohorts. The six cohorts were then combined using the random-effects model. The restricted maximum likelihood procedure was employed to estimate the between-cohort heterogeneity variance (τ²) using the R function MiMa (Viechtbauer, 2005, J Educ Behav Stat, 30: 261-293) (www.wvbauer.com). From this estimate, the Cochran's Q statistic and the I² statistic (the percentage of variation across studies that is due to heterogeneity rather than chance) (Higgins et al., 2003, Br Med J, 327: 557-560) was calculated.

Bayesian Evaluation of the Strength of Association.

To evaluate the strength of association with intracranial aneurysm, a Bayesian approach (Wellcome Trust Case Control Consortium, 2007, Nature, 447: 661-678; Goodman, 1999, Ann Intern Med, 130: 1005-1013) was used. A limitation of the use of P values alone is that variability in factors such as effect size, MAF and sample size can result in identical statistics that might correspond to markedly different levels of evidence regarding the strength of association (Stephens et al., 2009, Nat Rev Genet, 10: 681-690). The Bayes factor provides an alternative that compares the probabilities of the data under the alternative hypothesis of association versus the null hypothesis of no association. For computational simplicity, the Bayes factor was approximated as described by Wakefield (Wakefield, 2007, Am J Hum Genet, 81: 208-227), For all SNPs, the same prior distribution for the log-OR was assumed: a normal distribution with a mean of 0 and a standard deviation of log(1.5)/Φ⁻¹(0.975), where Φ is the normal distribution function (Wellcome Trust Case Control Consortium, 2007, Nature, 447: 661-678).

The PPA (Stephens et al., 2009, Nat Rev Genet, 10: 681-690) provides a simple probabilistic measure of evidence by introducing the prior probability of association, π₁. A uniform prior, π₁=1/10,000, was assumed for all the SNPs (Wacholder et al., 2004, J Natl Cancer Inst, 96: 434-442). For Bayes factor>10⁶, changing π₁ to a more conservative value of 1/100,000 would result in little change in the PPA.

To combine the results from multiple cohorts, the formula (Wakefield, 2008, Int J Epidemiol, 37: 641-653) was extended to be applicable to multiple (>2) cohorts.

Conditional Analysis.

For each region that contained a SNP with PPA>0.5, the number of independent association signals was examined by testing for association of every genotyped SNP with intracranial aneurysm by adjusting for the effect of a specified SNP (Table 10).

Two-Locus Interaction Analysis.

Deviation from a linear model, which assumes that two SNPs combine to increase the log-odds of disease in an additive fashion, was tested for using conditional (in the Finnish and European cohorts) or unconditional (in JP1 plus JP2, stratified by cohorts and gender) logistic regression. There was no significant deviation from the linear model.

Cumulative Effect.

Potential clinical implications of the genetic profiles of the five intracranial aneurysm risk loci were evaluated following the approach described previously (Clayton, 2009, PLoS Genet, 5: e1000540). A five-locus conditional (Finnish and European cohorts) or unconditional (Japanese cohorts) logistic regression model was fitted including the additive and dominance-deviation terms for each locus. Using the estimated effect sizes and each individual's genotype, the risk scores for every individual was calculated. The receiver-operating characteristic curve for each ethnic cohort (Finnish, European and Japanese) was depicted using the risk score.

The ratio of the exponential of the mean of the risk scores for control subjects within the top versus bottom 5% or 1% tails of distribution of risk scores in each cohort was also calculated to obtain approximated odds ratios of disease between these classes.

The sibling recurrence risk was estimated by assuming the polygenic model that fits well to the data (Clayton, 2009, PLoS Genet, 5: e1000540). A fraction of the sibling recurrence risk attributable to all of the five loci was calculated by taking the ratio of the logarithm of this value and epidemiologically estimated value of 4 (Schievink, 1997, Neurosurgery, 40: 651-663; Cannon Albright et al., 2003, J Neurosurg, 99: 637-643)

The results of the experiments are now described,

Intracranial aneurysms affect approximately 2% of the general population and arise from the action of multiple genetic and environmental risk factors (Rinkel et al., 1998, Stroke, 29: 251-256). A first genome-wide association study (GWAS) of intracranial aneurysms (Bilguvar et al., 2008, Nat Genet, 40: 1472-1477) was previously reported, which identified three risk loci on chromosomes 8q11.23-q12.1, 9p21.3 and 2q33.1 with P<5×10⁻⁸. This previous study had limited power to detect loci imparting genotypic relative risk (GRR)<1.35 (Table 10).

To increase the power to detect additional loci of similar or smaller effect, two new European case cohorts (n=1,616) were ascertained and whole-genome genotyped. Also, genotyping data from five additional European control cohorts (n=11,955) was collected. The size of the original Japanese replication cohort was increased and a new one (2,282 affected individuals (cases) and 905 controls) was added (Table 8). The new combined cohort had nearly threefold more cases than the original cohort and increased the power to detect variants with modest effect sizes. For example, this study had 89% and 64% average power to detect common variants (minor allele frequencies (MAF)≧10%) with GRR of 1.25 and 1.20, respectively (Table 10).

All subjects were genotyped using the Illumina platform. The new as well as the previously analyzed genotyping data were subjected to well-established quality-control measures (Table 11 and Table 12). It was sought to eliminate potential confounding due to population stratification and gender (Rinkel et al., 1998, Stroke, 29: 251-256; Iwamoto et al., 1999, Stroke, 30: 1390-1395) by matching cases and controls of the same gender based on inferred genetic ancestry. As previous studies demonstrated that the Finnish population forms an ancestry cluster distinct from other European populations similar to those included in this study (Salmela et al., 2008, PLoS One, 3: e3519; Jakkula et al., 2008, Am J Hum Genet, 83: 787-794), the Finnish cohort was analyzed independently from the others. To maximize opportunities for genetic matching and analytic power, all subjects in the remaining European cohorts were analyzed together. The resulting matched case-control data consisted of 808 cases and 4,393 controls in the Finnish cohort and 1,972 cases and 8,122 controls in the rest of the combined European cohort (Table 13). The genotype data that passed quality-control filters and phased chromosomes from the HapMap CEU sample was used to impute missing genotypes (Marchini et al., 2007, Nat Genet, 39: 906-913). Further analyses were based on 831,534 SNPs that passed the quality-control filters both in the Finnish and European samples (Table 8 and Table 12).

Association of each quality control-passed SNP with intracranial aneurysms was tested for using conditional logistic regression, assuming a log-additive effect of allele dosage. Each cohort was corrected for residual overdispersion (Table 8) using genomic control (Devlin et al., 1999, Biometrics, 55: 997-1004) and the results from the Finnish and European cohorts were combined to obtain P values, ORs and CIs for the discovery cohort of 2,780 cases and 12,515 controls using a fixed-effects model.

To evaluate the strength of association, in addition to obtaining P values, a Bayesian approach (Wakefield, 2007, Am J Hum Genet, 81: 208-227) was employed. The Bayes factor that represents the fold-change of the odds of association before and after observing the data (Wellcome Trust Case Control Consortium, 2007, Nature, 447: 661-678) and the posterior probability of association (PPA), calculated through the Bayes factor, that provides a simple probabilistic measure of the evidence of association (Wakefield, 2007, Am J Hum Genet, 81: 208-227; Stephens et al., 2009, Nat Rev Genet, 10: 681-690) was used. For every SNP, a uniform prior probability of association of 1/10,000 was assumed and the prior of the logarithm of the per-allele OR was set as a normal distribution with a 95% probability for the OR to be between 0.67 and 1.5, with larger weights for smaller effect sizes (Wellcome Trust Case Control Consortium, 2007, Nature, 447: 661-678; Wacholder et al., 2004, J Natl Cancer Inst, 96: 434-442).

From the discovery results, two imputed SNPs that showed PPAs of 0.97 and 0.94 were eliminated because their association signals were not supported by surrounding genotyped SNPs and because their genotypes were not confirmed by direct genotyping results (data not shown). This resulted in 831,532 SNPs that passed quality control (Table 12).

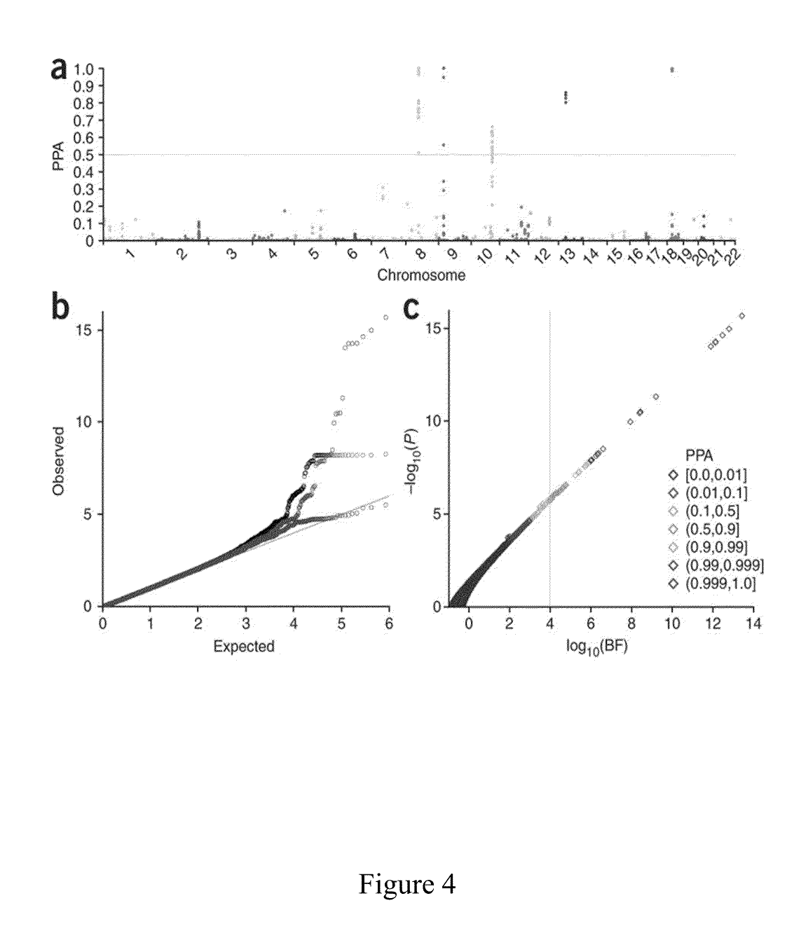

Three regions that showed very high PPAs were observed (>0.995; FIG. 4A). These regions also showed a substantial excess of SNPs with P<1×10⁻³ (1,295 SNPs versus 831 SNPs expected by chance) even after excluding those within previously identified associated regions (Bilguvar et al., 2008, Nat Genet, 40: 1472-1477) (FIG. 4B). Moreover, a strong correlation between the P values and Bayes factors was observed for the upper tail of the distribution (FIG. 4C).

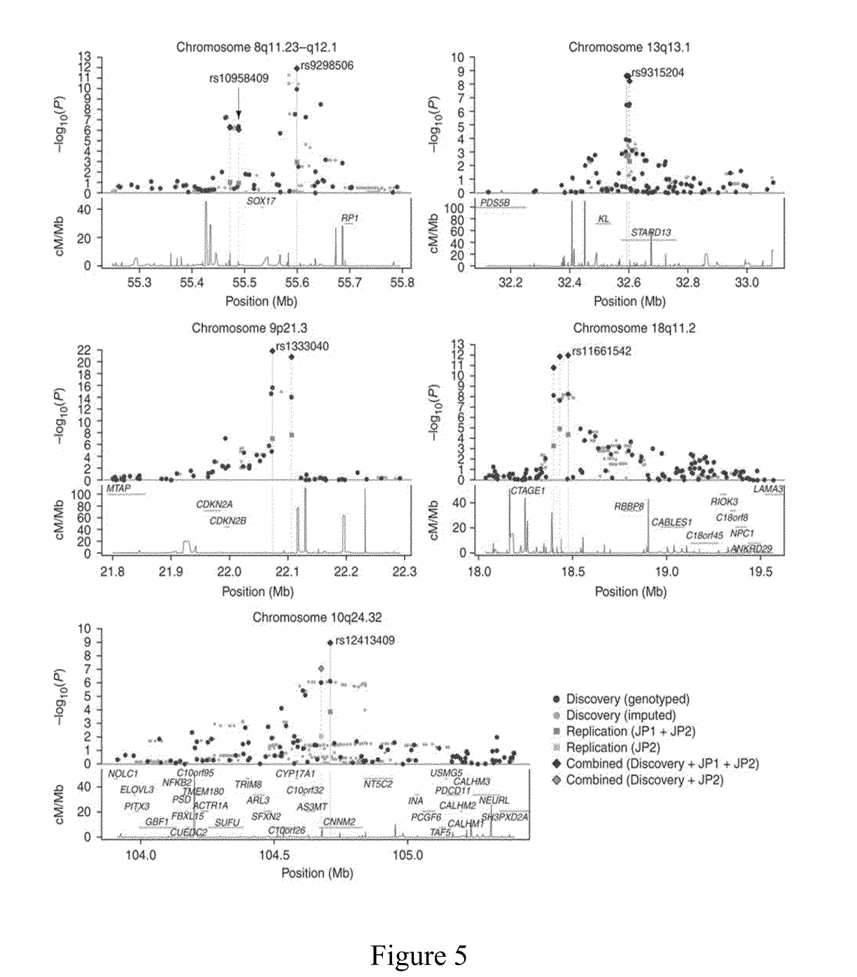

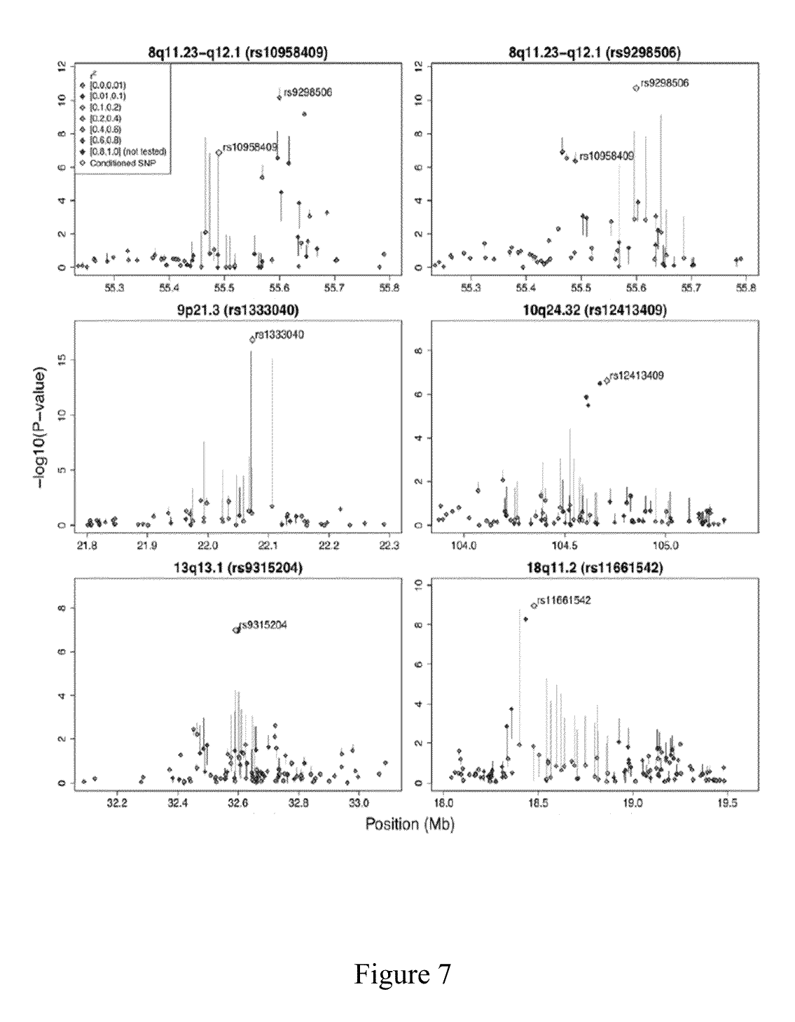

Five genomic regions (FIG. 4A) that contained at least one SNP with PPA>0.5 was focused upon, for which the hypothesis of association with intracranial aneurysm was more likely than the null hypothesis of no association. The PPAs of the most highly associated SNPs in these intervals ranged from 0.6621 to >0.9999 and the values ranged from 7.9×10⁻⁷ to 2.2×10⁻¹⁶ (Table 14). The five chromosomal segments included three newly identified SNP clusters on 10q24.32, 13q13.1 and S8q11.2. The remaining two regions were previously identified loci on 8q11.23-12.1 and 9p21.3 (Bilguvar et al., 2008, Nat Genet, 40: 1472-1477) (FIG. 5). The third locus identified in the previous study, on 2q33, did not contain any SNPs with PPA>0.5. Furthermore, consistent with previous results (Bilguvar et al., 2008, Nat Genet, 40: 1472-1477), detailed analysis of the 8q11.23-q12.1 region detected two independent association signals within the <100-kb interval that spans the SOX17 locus (FIG. 5 and FIG. 7); these two signals are hereafter referred to as 5′-SOX17 and 3′-SOX17. Thus, the five chromosomal segments comprised six independent association signals for follow-up.

Replication genotyping was performed in two Japanese cohorts including 3,111 cases and 1,666 controls (JP1 and JP2, see Table 8). For each independent signal, the genotyped SNP with the highest PPA was selected for replication and up to two additional SNPs per locus were added. For the 5′-SOX17 region, two SNPs analyzed previously were selected, as they tag the most significant SNP in the current study (FIG. 7).

All but one of the SNPs (rs12411886 on 10q24.32 in JP1) were successfully genotyped and passed quality-control filters, Association of each SNP with intracranial aneurysm was tested for using logistic regression stratified by gender, specifying the same model as for the discovery cohort (Table 15). Results from JP1 and JP2 were combined using a fixed-effects model (Table 9 and Table 14). An association was considered to be replicated if the Bayes factor increased the odds of association more than tenfold after the replication data was observed.

Of the six candidate loci, all but the 5′-SOX17 interval were replicated, with replication P values ranging from 0.0019 to 1.0×10⁻⁷, and the odds of association with intracranial aneurysm increasing by 22.9-fold to 1.5×10⁵-fold, yielding robust evidence for replication for each interval (Table 9).

The discovery and replication results were combined using a fixed-effects model. All of the five loci that replicated in the Japanese cohort surpassed the conventional threshold for genome-wide significance (P<5×10⁻⁸), with P values ranging from 2.5×10⁻⁹ to 1.5×10⁻²², and all also had PPAs≧0.998 (Table 9).

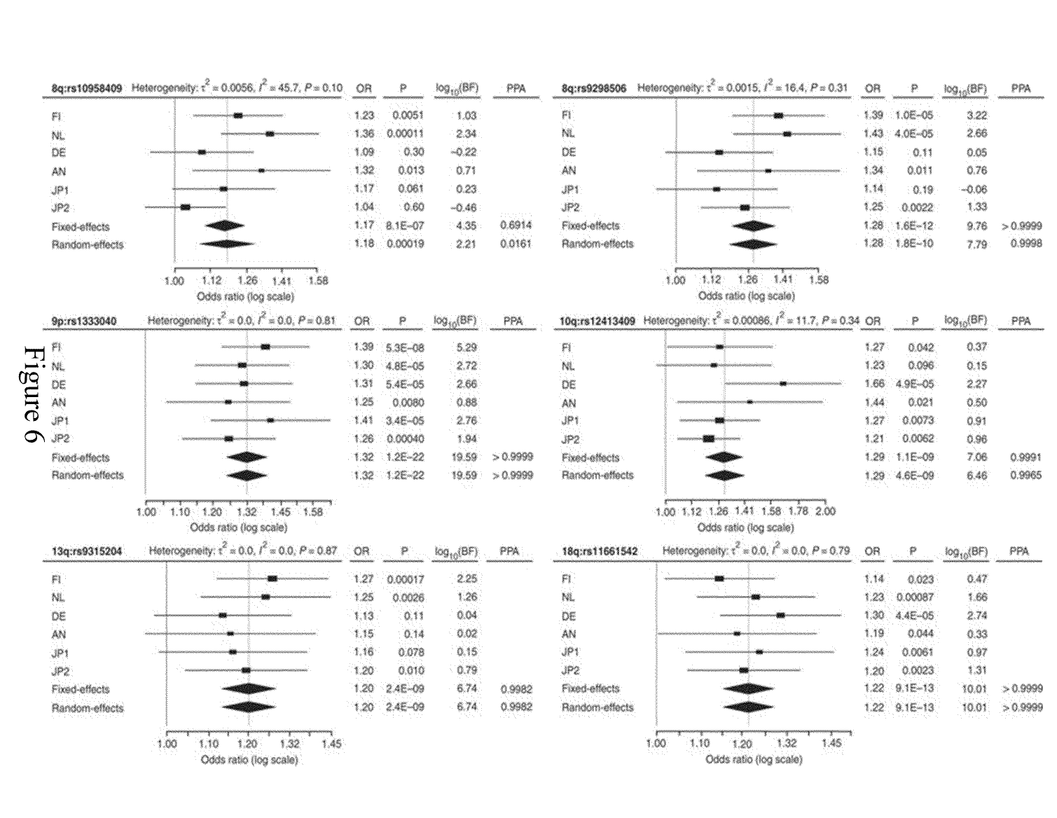

In order to determine each cohort's contribution to the observed association and to assess the consistency of the effect size across groups, each of them was analyzed separately (Table 8 and Table 15) and the results from the six cohorts were then combined using a random-effects model. The association results remained highly significant (FIG. 6). For the five loci that were replicated in the Japanese cohorts, no evidence of significant heterogeneity was found (P>0.1). Every cohort had the same risk allele and provided support for association with the exception of the JP1 sample for the 3′-SOX17 locus, consistent with the previous study (Bilguvar et al., 2008, Nat Genet, 40: 1472-1477) (FIG. 6).

The most significant association was detected in the previously reported (Bilguvar et al., 2008, Nat Genet, 40: 1472-1477) 9p21.3 region near CDKN2A and CDKN2B with P=1.5×10⁻²² (OR=1.32, PPA>0.9999). All of the newly studied cohorts strongly supported this association with intracranial aneurysm (FIG. 6). The same allele is associated with coronary artery disease but not with type 2 diabetes (Helgadottir et al., 2008, Nat Genet, 40: 217-224). Similarly, the previously reported 8q11.23-q12.1 region showed significant association. The 3′-SOX17 interval (rs92986506) showed robust association with P=1.3×10⁻² (OR=1.28, PPA>0.9999) and all new cohorts supported the association of this SNP with intracranial aneurysm (FIG. 6). For the 5′-SOX17 region (rs10958409), the new cohorts introduced a substantial heterogeneity, lowering the PPA to 0,016 (FIG. 6).

Among the newly identified loci, the strongest association was found at rs11661542 on 8q11.2 (OR=1.22, P=1.1×10⁻¹², PPA>0.9999). A cluster of SNPs that is associated with intracranial aneurysm spans the interval between 18.400 Mb and 18.509 Mb and is strongly correlated with rs11661542 (FIG. 5). A single gene, RBBP8 (encoding the retinoblastoma binding protein 8), is located within an extended linkage disequilibrium interval (FIG. 5).

The second strongest new association was at rs12413409 on 10q24.32 (OR=1.29, P=1.2×10⁻⁹, PPA=0.9990), which maps to intron 1 of CNNM2 (encoding cyclin M2) (FIG. 5). A cluster of SNPs that are strongly correlated with rs12413409 and are located within a ˜247-kb interval in the same linkage disequilibrium block supported the association (FIG. 5).

The third new locus is defined by rs9315204 at 13q13.1 (OR=1.20, P=2.5×10⁻⁹, PPA=0.9981) in intron 7 of STARD13 (encoding the StAR-related lipid transfer (START) domain containing 13) (FIG. 5). Two SNPs, rs1980781 and rs3742321, that are strongly correlated with rs9315204 (r²>0.9) also showed significant association with intracranial aneurysm (FIG. 5 and Table 4). These two SNPs are missense (lysine to arginine) and synonymous coding variants of STARD13, respectively. Another gene that has been implicated in aging phenotypes, KL (encoding klotho), is located nearby (Kuro-o et al., 1997, Nature, 390: 45-5).

A search of the gene-expression database (eQTL browser, eqtl.uchicago.edu) for all the intracranial aneurysm-risk loci did not reveal any consistent pattern of association of intracranial aneurysm SNPs with variation in gene expression levels.

In this second GWAS of intracranial aneurysm, which included nearly three times as many cases as the initial study, three new risk loci were detected and strong independent evidence for association of two previously identified loci was obtained. The evidence that these are bona fide risk loci for intracranial aneurysm is very strong from both Bayesian measures and conventional P values.

Given the power (˜90%) to detect variants that confer risk of intracranial aneurysm with GRR=1.25 and MAFs≧10%, it is expected that most of these variants are identified, limited principally by potential gaps in SNP coverage. Indeed, across the rest of the genome, there was no locus with PPA>0.22 and MAF≧10%, whereas there were 14 loci with PPAs between 0.1 and 0.22 and ORs between 1.16 and 1.25 (data not shown). It is expected that a fraction of these loci are genuine intracranial aneurysm risk loci, as suggested by the excess of SNPs with P<1×10⁻³ (FIG. 4B); exploring this possibility will require analysis of larger intracranial aneurysm cohorts and/or genotyping of alleles with lower MAFs.

Based on the results of the first GWAS of intracranial aneurysm and the role of the implicated gene products, Sox17 and p15^{INK4b}-p16^{INK4a}, it was hypothesized that the genes associated with intracranial aneurysm might play a role in determining cell cycle progression and may affect the proliferation (Visel et al., 2010, Nature, 464: 409-412) and senescence of progenitor-cell populations and/or the balance between production of progenitor cells versus cells committed to differentiation. Without being bound to any particular theory, genes located within the newly identified regions support this idea. The protein encoded by RBBP8, located within the 18q11.2 region, influences progression through the cell cycle by interacting with BRCA1 (Yun et al., 2009, Nature, 459: 460-463). Similarly, of the two genes located within the 13q13.1 interval, STARD13 contains the Rho-GAP and C-terminal STAR-related lipid transfer (START) domains and its overexpression results in suppression of cell proliferation (Leung et al., 2005, Proc Natl Acad Sci USA, 102: 15207-15212). The other gene implicated here, KL, encodes a transmembrane protein that modulates FGF receptor specificity (Urakawa et al., 2006, Nature, 444: 770-774); mice lacking KL show accelerated aging in diverse organ systems (Kuro-o et al., 1997, Nature, 390: 45-51).

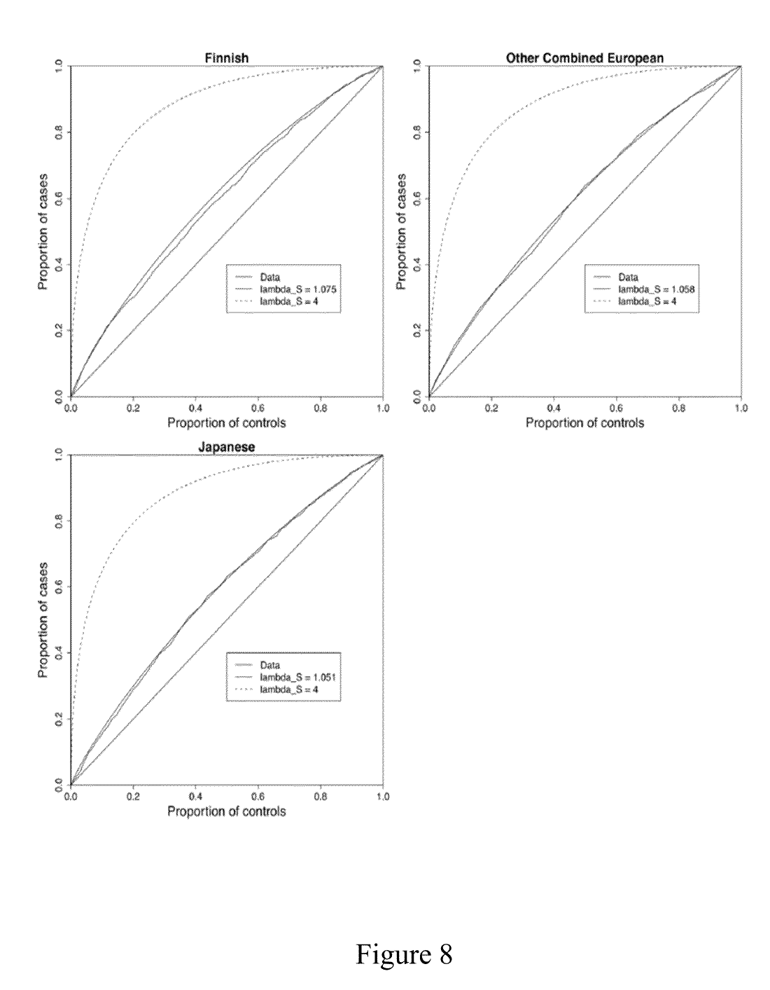

On the assumption that there is a four-fold increase in the risk of intracranial aneurysm among siblings of cases (Schievink, 1997, Neurosurgery, 40: 651-663; Cannon Albright et al., 2003, J Neurosurg, 99: 637-643) and that the SNPs combine to increase log-odds of disease in an additive fashion, the five intracranial aneurysm risk loci explain 5.2% (within the Finnish cohort), 4.0% (in the European cohort) and 3.5% (in the combined JP and JP2 cohort) of the familial risk of intracranial aneurysm. Under this model, the odds of developing an intracranial aneurysm varies 4.99- to 7.63-fold across the top and bottom 1% of genetic risk profiles at these loci in the populations studied here and 3.61- to 4.64-fold across the 5% extremes (FIG. 8). When combined with traditional risk factors such as gender, blood pressure and smoking, these findings permit the preclinical identification of individuals who are at high risk of intracranial aneurysm formation and rupture.

TABLE 8
Overview of the study cohorts

| | | Case | Control | Quality control- | |
|---|---|---|---|---|---|
| | Cohort | (n) | (n) | passed SNPs (n) | GIF |
| Discovery | Finland (FI) | 808 | 4,393 | 1,303,876 | 1.074 |
| | Combined | 1,972 | 8,122 | 905,906 | 1.094 |
| | European (CE) | | | | |
| | Total discovery | 2,780 | 12,515 | 831,532 | 1.007 |
| CE subcohorts | NL | 708 | 3,954 | 905,906 | 1.108 |
| | DE | 789 | 2,228 | 905,906 | 1.059 |
| | AN | 475 | 1,940 | 905,906 | 1.057 |
| Replication | Japan 1 (JP1) | 829 | 761 | 12 | |
| | Japan 2 (JP2) | 2,282 | 905 | 13 | |
| | Total replication | 3,111 | 1,666 | 12 | |
| | Total | 5,891 | 14,181 | 12 | |

Combined European cohort consisted of all European subjects who were not ascertained in Finland. Sub-cohorts of the European cohort were defined on the basis of case series; NL, cases from The Netherlands with matched controls; DE, German cases with matched controls; AN, @neurIST cases with matched controls. NL, DE and AN were exclusive subsets of the European cohort (see also Table 13). AN cases consisted of subjects from Germany, Great Britain, Hungary, The Netherlands, Switzerland and Spain. JP1 and JP2 were two independent Japanese case-control cohorts. Genomic inflation factors of the Finnish and European cohorts (as well as NL, DE and AN) were calculated for 1,303,876 and 905,906 SNPs, respectively. The genomic inflation factor of the discovery cohort (total discovery) was based on the meta-analysis result for 831,532 SNPs after correcting each cohort for genomic control. The discovery data (combined Finnish and European cohorts) was not corrected for genomic control. GIF, genomic inflation factor.

TABLE 9
Representative SNPs analyzed both in the discovery and replication cohorts

| | | | | Risk | | | | |
|---|---|---|---|---|---|---|---|---|
| Locus | SNP | Position | Genes | allele | Cohort | P value | log₁₀ (Bayes) | PPA |
| 8q11.23 | rs10958409 | 55,489,644 | SOX17 | A | Discovery | 4.2 × 10⁻⁷ | 4.64 | 0.8128 |
| | | | | | Replication | 0.12 | −0.11 | |
| | | | | | Combined | 9.0 × 10⁻⁷ | 4.30 | 0.6685 |
| 8q12.1 | rs9298506 | 55,600,077 | SOX17 | A | Discovery | 1.2 × 10⁻¹⁰ | 7.94 | 0.9999 |
| | | | | | Replication | 0.0012 | 1.56 | |
| | | | | | Combined | 1.3 × 10⁻¹² | 9.85 | 1.0-1.4 × 10⁻⁶ |
| 9p21.3 | rs1333040 | 22,073,404 | CDKN2A, | T | Discovery | 2.5 × 10⁻¹⁶ | 13.41 | 1.0-3.9 × 10⁻¹⁰ |
| | | | CDKN2B | | Replication | 1.0 × 10⁻⁷ | 5.18 | |
| | | | | | Combined | 1.5 × 10⁻²² | 19.48 | 1.0-3.3 × 10⁻¹⁶ |
| 10q24.32 | rs12413409 | 104,709,086 | CNNM2 | G | Discovery | 7.9 × 10⁻⁷ | 4.29 | 0.6621 |
| | | | | | Replication | 0.00014 | 2.34 | |
| | | | | | Combined | 1.2 × 10⁻⁹ | 7.00 | 0.9990 |
| 13q13.1 | rs9315204 | 32,691,837 | KL, STARD13 | T | Discovery | 3.3 × 10⁻⁷ | 4.73 | 0.8443 |
| | | | | | Replication | 0.0019 | 1.36 | |
| | | | | | Combined | 2.5 × 10⁻⁹ | 6.72 | 0.9981 |
| 18q11.2 | rs11661542 | 18,477,693 | RBBP8 | C | Discovery | 5.6 × 10⁻⁹ | 6.39 | 0.9959 |
| | | | | | Replication | 4.5 × 10⁻⁵ | 2.79 | |
| | | | | | Combined | 1.1 × 10⁻¹² | 9.92 | 1.0-1.2 × 10⁻⁶ |

| | | | | Risk | | Per-allele OR | | |
|---|---|---|---|---|---|---|---|---|
| Locus | SNP | Position | Genes | allele | Cohort | (95% CI) | Control RAF | Case RAF |
| 8q11.23 | rs10958409 | 55,489,644 | SOX17 | A | Discovery | 1.24 (1.14-1.35) | 0.15, 0.19 | 0.10, 0.22 |
| | | | | | Replication | 1.08 (0.98-1.20) | 0.28 | 0.29 |
| | | | | | Combined | 1.17 (1.10-1.26) | | |
| 8q12.1 | rs9298506 | 55,600,077 | SOX17 | A | Discovery | 1.33 (1.22-1.45) | 0.81, 0.76 | 0.85, 0.81 |
| | | | | | Replication | 1.21 (1.08-1.36) | 0.79 | 0.81 |
| | | | | | Combined | 1.28 (1.20-1.38) | | |
| 9p21.3 | rs1333040 | 22,073,404 | CDKN2A, | T | Discovery | 1.32 (1.24-1.41) | 0.56, 0.45 | 0.63, 0.53 |
| | | | CDKN2B | | Replication | 1.31 (1.19-1.45) | 0.66 | 0.72 |
| | | | | | Combined | 1.32 (1.25-1.39) | | |
| 10q24.32 | rs12413409 | 104,709,086 | CNNM2 | G | Discovery | 1.38 (1.22-1.57) | 0.91, 0.91 | 0.94, 0.93 |
| | | | | | Replication | 1.23 (1.10-1.37) | 0.74 | 0.77 |
| | | | | | Combined | 1.29 (1.19-1.40) | | |
| 13q13.1 | rs9315204 | 32,691,837 | KL, STARD13 | T | Discovery | 1.21 (1.13-1.31) | 0.21, 0.33 | 0.24, 0.39 |
| | | | | | Replication | 1.18 (1.06-1.31) | 0.24 | 0.27 |
| | | | | | Combined | 1.20 (1.13-1.28) | | |
| 18q11.2 | rs11661542 | 18,477,693 | RBBP8 | C | Discovery | 1.21 (1.14-1.30) | 0.49, 0.44 | 0.54, 0.47 |
| | | | | | Replication | 1.22 (1.11-1.34) | 0.61 | 0.65 |
| | | | | | Combined | 1.22 (1.15-1.28) | | |

Genomic locations for SNPs are based on NCBI build 36, and risk alleles are aligned to the forward strand of the reference sequence. Control and case risk allele frequencies (RAFs) for the discovery cohort are shown in the form: RAF of European cohort, RAF of Finnish cohort. Log₁₀ (Bayes) indicates the logarithm of the Bayes factor in favor of association. PPA, posterior probability of association. Genes closest to the listed SNPs within the same LD regions are shown.

TABLE 10
Power estimates for the previous and current studies

| | Previous Study: mean (range) | | | Current Study: mean (range) | | |
|---|---|---|---|---|---|---|
| GRR | Discovery | Replication | Combined | Discovery | Replication | Combined |

0.05 ≦ RAF ≦ 0.95 in 3 cohorts

| | | | | | | |
|---|---|---|---|---|---|---|
| 1.2 | 0.15 (0.00-0.37) | 0.15 (0.00-1.00) | 0.05 (0.00-0.23) | 0.63 (0.00-0.97) | 0.70 (0.00-1.00) | 0.56 (0.00-0.96) |
| 1.25 | 0.39 (0.00-0.76) | 0.28 (0.00-1.00) | 0.23 (0.00-0.62) | 0.83 (0.01-1.00) | 0.85 (0.00-1.00) | 0.80 (0.00-1.00) |
| 1.3 | 0.62 (0.00-0.94) | 0.42 (0.00-1.00) | 0.46 (0.00-0.92) | 0.91 (0.08-1.00) | 0.91 (0.25-1.00) | 0.90 (0.03-1.00) |
| 1.35 | 0.77 (0.00-1.00) | 0.54 (0.00-1.00) | 0.66 (0.00-0.99) | 0.96 (0.21-1.00) | 0.95 (0.38-1.00) | 0.95 (0.14-1.00) |

0.1 ≦ RAF ≦ 0.9 in 3 cohorts

| | | | | | | |
|---|---|---|---|---|---|---|
| 1.2 | 0.17 (0.00-0.37) | 0.18 (0.00-1.00) | 0.06 (0.00-0.23) | 0.70 (0.09-0.97) | 0.77 (0.11-1.00) | 0.64 (0.01-0.96) |
| 1.25 | 0.44 (0.01-0.76) | 0.32 (0.00-1.00) | 0.27 (0.00-0.62) | 0.91 (0.29-1.00) | 0.91 (0.49-1.00) | 0.89 (0.21-1.00) |
| 1.3 | 0.69 (0.06-0.94) | 0.46 (0.00-1.00) | 0.54 (0.01-0.92) | 0.97 (0.53-1.00) | 0.97 (0.65-1.00) | 0.97 (0.44-1.00) |
| 1.35 | 0.84 (0.10-1.00) | 0.60 (0.07-1.00) | 0.75 (0.04-0.99) | 0.99 (0.75-1.00) | 0.99 (0.80-1.00) | 0.99 (0.70-1.00) |

0.2 ≦ RAF ≦ 0.8 in 3 cohorts

| | | | | | | |
|---|---|---|---|---|---|---|
| 1.2 | 0.21 (0.05-0.37) | 0.21 (0.00-0.54) | 0.09 (0.00-0.23) | 0.82 (0.48-0.97) | 0.85 (0.58-0.99) | 0.78 (0.38-0.96) |
| 1.26 | 0.54 (0.22-0.76) | 0.38 (0.07-0.64) | 0.35 (0.08-0.82) | 0.98 (0.80-1.00) | 0.97 (0.82-1.00) | 0.97 (0.77-1.00) |
| 1.3 | 0.80 (0.37-0.94) | 0.54 (0.19-0.81) | 0.67 (0.20-0.92) | 1.00 (0.97-1.00) | 0.09 (0.93-1.00) | 1.00 (0.96-1.00) |
| 1.35 | 0.94 (0.74-1.00) | 0.69 (0.35-0.90) | 0.87 (0.56-0.99) | 1.00 (1.00-1.00) | 1.00 (0.99-1.00) | 1.00 (0.99-1.00) |

A multiplicative model of genotypic relative risk (GRR) of a causal SNP and intracranial aneurysm prevalence of 2% were assumed (Rinkel at al., 1998, Stroke, 29: 251-256). Sample size of the previous study (Bilguvar et al., 2008, Nat Genet, 40: 1472-1477) (case; control): Finnish = (874; 944); Dutch = (706; 5,332); Japanese = (495; 676). Sample size of the current study: Finnish = (808; 4,393); other combined European = (1,972; 8,122); Japanese = (3,111; 1,666). Risk allele frequencies (RAFs) of 3 cohorts were selected from 5% to 95% by increments of 5% but restricted to keep the maximum frequency difference between Finnish and other European cohorts to less than 10%. Similarly, maximum RAF difference between Japanese and one of the European (Finnish or other European) cohorts is kept less than 40%. These maximum differences were determined from the empirical distribution of allele frequencies of genotyped SNPs on chromosome 21 and contained approximately 95% of the SNPs. For each cohort, genotype counts of cases and controls were simulated using the prevalence, RAF in the population and GRR. The logarithm of per-allele odds ratio and the standard error was then estimated using the logistic regression. To combine Bayes factors (BFs) and posterior probabilities of association (PPAs) were calculated as described previously (Wakefield, 2007: Am J Hum Genet, 81: 208-227). Power to detect association was estimated as the number of times the simulated result meets the criteria given below divided by the number of simulations which was 100.
Discovery = PPA >0.5 in the discovery cohort (Finnish + Dutch/other European);
Replication = Conditional on Discovery, BF >10 in the Japanese cohort with the same risk allele as that of the discovery cohort;
Combined = Discovery, PPA >0.99 in the joint analysis of discovery and replication cohorts and PPA in the joint analysis greater than that in the discovery cohort.

TABLE 11
Sample quality control process

| | | | | | | Excluded subjects‡ | | | | | | |
|---|---|---|---|---|---|---|---|---|---|---|---|---|
| | | | | | | Genotyping | | | Nearest | | | |
| | | | | | | And | | Cryptic | From | Popula- | | |
| | | | | Cohort | Initial | Information | Spurious | Related- | Many | tion | Not | Final |
| Cohort' | Country | Beadchlp | Prev† | Type | Size | Quality | Relatedness | ness | Subjects | Outliers | Matched | Size |
| FI | Finland | CNV370 | Y | case | 912 | 8 | 0 | 66 | 0 | 3 | 27 | 808 |
| case control | | | | control | 740 | 0 | 0 | 85 | 1 | 16 | 38 | 600 |
| Heath2000 | Finland | Human610 | N | control | 2,138 | 22 | 0 | 66 | 3 | 31 | 273 | 1,743 |
| NFBC1966 | Finland | CNV370 | N | control | 5,302 | 104 | 0 | 1,295 | 4 | 24 | 1,825 | 2,060 |
| HL cases | The | CNV370 | Y | case | 786 | 20 | 14 | 23 | 0 | 21 | 0 | 708 |
| | Netherlands | | | | | | | | | | | |
| Utrecht | The | Hap300v1 | Y | control | 450 | 7 | 1 | 22 | 1 | 5 | 8 | 408 |
| | Netherlands | | | | | | | | | | | |
| RS | The | Hap550v3 | Y | control | 5,974 | 33 | 1 | 1,435 | 1 | 31 | 62 | 4,411 |
| | Netherlands | | | | | | | | | | | |
| DE cases | Germany | Human610 | N | case | 975 | 33 | 3 | 132 | 0 | 15 | 3 | 789 |
| KORA-gen | Germany | Hap550v3 | N | control | 840 | 14 | 0 | 19 | 1 | 6 | 5 | 795 |
| PopGen | Germany | Hap550v3 | N | control | 493 | 2 | 0 | 11 | 0 | 3 | 8 | 469 |
| @neurIST | see below | Human610 | N | case | 641 | 21 | 6 | 71 | 0 | 43 | 25 | 475 |
| IcontrolDB | undown | Hap550v1/v3 | N | control | 3,182 | 30 | 8 | 107 | 1 | 478 | 519 | 2,039 |

*Health2000 = Finland Health 2000;
NFBC1966 = Northern Finnish Birth Cohort 1966;
Utrecht = Utrecht neurologically normal subjects;
RS = The Rotterdam study.
@neurIST sample consists of subjects from Germany, Great Britain, Hungary, The Netherlands, Switzerland and Spain,
†The column indicates whether the cohort was analyzed in the previous study (Bilguvar et al., 2008, Nat Genet, 40: 1472-1477) (Y = yes, N = no).
‡Number of excluded subjects were recorded for each step as described below:
Genotyping and information quality: Samples that were excluded from analyses were those that (a) were likely to be contaminated, which were identified by examining distribution of the normalized Theta (or B allele frequency) in the BeadStudio; (b) showed greater than 3% missing genotypes (missing rate >0.03); (c) belonged to a cluster showing systematically higher missing rates, indicating a batch effect; (d) showed discrepancy between stated and genotypic gender; (e) were outliers with respect to the estimated inbreeding coefficient (>0.125 or <−0.03), for which negatively large inbreeding coefficients indicate contamination, while positively large ones could be attributable to incomplete hybridization or to true inbreeding.
Spurious relatedness: Subjects who showed distant (approximately 3rd to 4th degree) relatedness with 5 or more subjects that were associated with high inbreeding coefficients were excluded. While this could be due to true relatedness, it is more likely to be due to long stretches of homozygosity resulting in spurious relationship between subjects.
Cryptic relatedness: The IBS similarity (the proportion of alleles shared identical-by-state (Purcell et al., 2007, Am J Hum Genet, 81: 559-575)) was estimated based on nearly independent SNPs extracted by using the complete-linkage hierarchical clustering (Rinaldo et al., 2005, Genet Epidemiol, 28: 193-206). Only one subject from duplicates or probable relatives was kept based on the IBS similarity.
Nearest-from-many subjects: Subjects that were indicated to be the nearest neighbor for more than 100 individuals was excluded as it suggested contamination.
Population outliers: Outliers were excluded based on empirical parameters for each cohort (CE and FI) which were identified using: (a) the mean IBS similarity calculated for each subject by averaging over all possible pairs; (b) the multidimensional scaling (MDS) analysis; (c) the maximum IBS similarity; and (d) the proportion of distant subjects.

TABLE 12
SNP quality control process

| | CE | | | | | FI | | | | |
|---|---|---|---|---|---|---|---|---|---|---|
| | | Genomic | | Genomic | | | Genomic | | Genomic | |
| | Remaining | inflation | Remaining | inflation | | Remaining | inflation | Remaining | inflation | |
| | genotyped | factor | Imputed | factor | | genotyped | factor | Imputed | factor | |
| | SNPs | (n with | SNPs | (n with | | SNPs | (n with | SNPs | (n with | |
| QC metric | (n) | P < 5 × 10⁻⁸) | (n) | P < 5 × 10⁻⁸) | | (n) | P < 5 × 10⁻⁸) | (n) | P < 5 × 10⁻⁸) | |
| Initial data | 294,374 | — | 3,859,875 | — | | 325,286 | — | 3,860,940 | — | |
| Imputation | — | — | 2,462,318 | 1.204 | (1,348) | — | — | 2,396,529 | 1.215 | (2,820) |
| quality | | | | | | | | | | |
| Genotyping | 278,285 | 1.099 (6) | 1,812,624 | 1.127 | (120) | 311,023 | 1.071 (2) | 1,808,313 | 1.114 | (145) |
| quality | | | | | | | | | | |
| Non-redundant | — | — | 1,652,092 | — | | — | — | 1,624,167 | — | |
| SNPs | | | | | | | | | | |
| Allelle R² ≧0.9 | — | — | 1,613,978 | 1.125 | (105) | — | — | 1,548,488 | 1.111 | (110) |
| Differential | 235,984 | 1.097 (6) | 669,922 | 1.092 | (30) | 296,849 | 1.069 (1) | 1,007,027 | 1.075 | (13) |
| missingness | | | | | | | | | | |
| Remaining | | 905,906 | | 1.094 | (36) | | 1303,876 | | 1.074 | (14) |
| SNPs in | | | | | | | | | | |
| each cohort | | | | | | | | | | |

| | |
|---|---|
| Remaining | 831,534 (genotyped = 233,749; Imputed = 597,785) |
| SNPs in | |
| both cohorts | |
| Final total | 831,532 (genotyped = 233,749; Imputed = 597,783) |

Initial data:
[Genotyped SNPs] Number of SNPs that were genotyped in at least one individual in every sub-cohort after excluding SNPs, as described elsewhere herein. For the sub-cohort definition, see Table 11).
[Imputed SNPs] All the SNPs analyzed using IMPUTE v1.0.0 software.
Imputation quality: MAF (based on fractional allele dosage score) ≧0.01, allelic R² (squared correlation between true and imputed genotypes) ≧0.3, and average posterior probability for the most likely genotype ≧0.9.
Genotyping quality: The following conditions had to be met in each of the cohorts,
[Genotyped SNPs] MAF ≧0.01, missing rate ≦0.005 if 0.01 ≦ MAF <0.05 or missing rate ≦0.05 if MAF ≧0.05, and P-value of HWE test ≧1 × 10⁻⁵.
[Imputed SNPs] (most likely genotypes with posterior probability ≧0.9 were used) MAF ≧0.01, missing rate ≦0.05 if 0.01 ≦MAF <0.05 or missing rate ≦0.1 if MAF ≧0.05, and P-value of HWE test ≧1 × 10⁻⁵.
Non-redundant SNPs: At this step, duplicated SNPs between genotyped and imputed ones were identified and the data from the imputation was excluded leading to a non-redundant, non-overlapping data set.
Allelic R2 ≧0.9: The allelic R² is defined as the squared correlation between true and imputed genotypes (Browning et al., 2009, Am J Hum Genet, 84: 210-223). This filter was added to keep only the imputed SNPs with high prediction accuracy, uniformly across all the cohorts.
Differential missingness: SNPs were eliminated if the absolute value of the difference of the missing rates between cases and controls >0.01 and P-value of the exact test of missingness <1 × 10⁻⁶. This stringent filter was added as the data consisted of cohorts genotyped using various Illumina beadchips.
Remaining SNPs in each cohort: The genotyped and imputed SNPs in each of the CE and FI cohorts were merged.
Remaining SNPs in both cohorts: SNPs that passed QC filters in both the CE and FI cohorts were extracted. If a SNP was genotyped in one cohort and imputed in the other one, this particular SNP was classified as imputed.
Final total: 2 imputed SNPs, in which association with intracranial aneurysm was implicated with PPA >0.5, were eliminated due to lack of supporting evidence from genotyped SNPs in LD with the imputed SNPs.

TABLE 13
Result of case-control matching

| | | | | | | | | | | Control |
|---|---|---|---|---|---|---|---|---|---|---|
| CE Case Series | | The Netherlands | | Germany | | iControlDB (Study ID) | | | | Total |
| (n = 1,972) | | Utrecht | RS | PopGen | KORA-gen | 64 | 65 | 66 | 67 | (#strata) |
| NL cases | 708 | 323 | 3,404 | 90 | 33 | 19 | 42 | 15 | 28 | 3,954 |
| | (35.9%) | (79.2%) | (77.2%) | (19.2%) | (4.2%) | (7.4%) | (6.7%) | (2.9%) | (4.4%) | (708) |
| DE cases | 789 | 56 | 600 | 341 | 579 | 82 | 174 | 179 | 217 | 2,228 |
| | (40.0%) | (13.7%) | (13.6%) | (72.7%) | (72.8%) | (32.0%) | (27.7%) | (34.6%) | (34.1%) | (786) |
| @neurIST | 476 | 29 | 407 | 38 | 183 | 155 | 413 | 324 | 391 | 1,940 |
| | (24.1%) | (7.1%) | (9.2%) | (8.1%) | (23.0%) | (60.5%) | (65.7%) | (62.5%) | (61.5%) | (452) |

| | | | | | | | | | |
|---|---|---|---|---|---|---|---|---|---|
| Cohort Total | 408 | 4,411 | 469 | 796 | 256 | 629 | 518 | 636 | 8,122 |

| | | | | | |
|---|---|---|---|---|---|
| | | | | | Control |
| FI Case Series | | | Health | NFBC | Total |
| (n = 808) | Helsinki | Kuoplo | 2000 | 1966 | (#strata) |

| | | | | | | |
|---|---|---|---|---|---|---|
| Helsinki | 510 | 265 | 92 | 1,206 | 1,295 | 2,858 |
| | (63.1%) | (75.5%) | (36.9%) | (69.2%) | (63.2%) | (510) |
| Kuoplo | 298 | 86 | 157 | 537 | 755 | 1,535 |
| | (36.9%) | (24.5%) | (63.1%) | (30.8%) | (36.8%) | (298) |

| | | | | | |
|---|---|---|---|---|---|
| Cohort Total | 351 | 249 | 1,743 | 2,050 | 4,393 |

Sub-cohorts of CE were defined based on the case series:
NL = NL cases (n = 708) and matched controls (n = 3,954);
DE = DE cases (n = 789) and matched controls (n = 2,228); and
AN = @neurIST cases (n = 475) and matched controls (n = 1,940).

TABLE 14
Summary of the results for 13 SNPs genotyped both in the discovery and
replication cohorts

| | | | | Risk | | | | |
|---|---|---|---|---|---|---|---|---|
| Locus | SNP | Position | Gene | Allele | Cohort | P-value | log₁₀(BF) | PPA |
| 8q11.23 | rs1504749 | 55473264 | 5′-SOX17 | C | Discovery | 4.6E−07 | 4.60 | 0.7984 |
| | | | | | Replication | 0.095 | −0.06 | |
| | | | | | Combined | 5.4E−07 | 4.50 | 0.7604 |
| 8q11.23 | rs10958409 | 55489644 | 5′-SOX17 | A | Discovery | 4.2E−07 | 4.64 | 0.8128 |
| | | | | | Replication | 0.12 | −0.11 | |
| | | | | | Combined | 9.0E−07 | 4.30 | 0.6685 |
| 8q12.1 | rs9298506 | 55600077 | 3′-SOX17 | A | Discovery | 1.2E−10 | 7.94 | 0.9999 |
| | | | | | Replication | 0.0012 | 1.58 | |
| | | | | | Combined | 1.3E−12 | 9.85 | 1.0-1.4E−06 |
| 9p21.3 | rs1333040 | 22073404 | CDKN2A/B | T | Discovery | 2.5E−16 | 13.41 | 1.0-3.9E−10 |
| | | | | | Replication | 1.0E−07 | 5.18 | |
| | | | | | Combined | 1.5E−22 | 19.48 | 1.0-3.3E−16 |
| 9p21.3 | rs2383207 | 22105959 | CDKN2A/B | G | Discovery | 9.7E−15 | 11.89 | 1.0-1.3E−08 |
| | | | | | Replication | 2.6E−08 | 5.74 | |
| | | | | | Combined | 1.5E−21 | 18.51 | 1.0-3.1E−15 |
| 10q24.32 | rs12411886 | 104675289 | CNNM2 | C | Discovery | 1.0E−06 | 4.19 | 0.6089 |
| | | | | | Replication (JP2) | 0.0092 | 0.82 | |
| | | | | | Combined | 9.5E−08 | 5.22 | 0.9437 |
| 10q24.32 | rs12413409 | 104709086 | CNNM2 | G | Discovery | 7.9E−07 | 4.29 | 0.6621 |
| | | | | | Replication | 0.00014 | 2.34 | |
| | | | | | Combined | 1.2E−09 | 7.00 | 0.9990 |
| 13q13.1 | rs9315204 | 32591837 | STARD13 | T | Discovery | 3.3E−07 | 4.73 | 0.8443 |
| | | | | | Replication | 0.0019 | 1.38 | |
| | | | | | Combined | 2.5E−09 | 6.72 | 0.9981 |
| 13q13.1 | rs1960781 | 32598374 | STARD13 | G | Discovery | 3.8E−07 | 4.68 | 0.8259 |
| | | | | | Replication | 0.0018 | 1.39 | |
| | | | | | Combined | 2.6E−09 | 6.70 | 0.9980 |
| 13q13.1 | rs3742321 | 32602065 | STARD13 | C | Discovery | 2.9E−07 | 4.79 | 0.8597 |
| | | | | | Replication | 0.0049 | 1.01 | |
| | | | | | Combined | 6.1E−09 | 6.35 | 0.9955 |
| 18q11.2 | rs4800418 | 18400738 | RBBP8 | C | Discovery | 7.8E−09 | 6.26 | 0.9945 |
| | | | | | Replication | 0.00054 | 1.84 | |
| | | | | | Combined | 1.7E−11 | 8.78 | 1.0-1.7E−05 |
| 18q11.2 | rs11662668 | 18433379 | RBBP8 | G | Discovery | 2.4E−08 | 5.80 | 0.9844 |
| | | | | | Replication | 1.2E−05 | 3.29 | |
| | | | | | Combined | 1.4E−12 | 9.81 | 1.0-1.5E−06 |
| 18q11.2 | rs11661542 | 18477693 | RBBP8 | C | Discovery | 5.6E−09 | 6.39 | 0.9959 |
| | | | | | Replication | 4.5E−05 | 2.79 | |
| | | | | | Combined | 1.1E−12 | 9.92 | 1.0-1.2E−06 |

| | | | | Risk | | | Control | Case |
|---|---|---|---|---|---|---|---|---|
| Locus | SNP | Position | Gene | Allele | Cohort | OR (95% CI) | RAF | RAF |
| 8q11.23 | rs1504749 | 55473264 | 5′-SOX17 | C | Discovery | 1.22 (1.13-1.31) | 0.21/0.24 | 0.24/0.27 |
| | | | | | Replication | 1.09 (0.99-1.20) | 0.30 | 0.33 |
| | | | | | Combined | 1.17 (1.10-1.24) | | |
| 8q11.23 | rs10958409 | 55489644 | 5′-SOX17 | A | Discovery | 1.24 (1.14-1.35) | 0.15/0.19 | 0.18/0.22 |
| | | | | | Replication | 1.08 (0.98-1.20) | 0.28 | 0.29 |
| | | | | | Combined | 1.17 (1.10-1.25) | | |
| 8q12.1 | rs9298506 | 55600077 | 3′-SOX17 | A | Discovery | 1.33 (1.22-1.45) | 0.81/0.76 | 0.85/0.81 |
| | | | | | Replication | 1.21 (1.08-1.36) | 0.79 | 0.81 |
| | | | | | Combined | 1.28 (1.20-1.38) | | |
| 9p21.3 | rs1333040 | 22073404 | CDKN2A/B | T | Discovery | 1.32 (1.24-1.41) | 0.56/0.45 | 0.63/0.53 |
| | | | | | Replication | 1.31 (1.19-1.45) | 0.66 | 0.72 |
| | | | | | Combined | 1.32 (1.25-1.39) | | |
| 9p21.3 | rs2383207 | 22105959 | CDKN2A/B | G | Discovery | 1.29 (1.21-1.38) | 0.50/0.43 | 0.58/0.50 |
| | | | | | Replication | 1.32 (1.20-1.45) | 0.63 | 0.70 |
| | | | | | Combined | 1.30 (1.23-1.37) | | |
| 10q24.32 | rs12411886 | 104675289 | CNNM2 | C | Discovery | 1.38 (1.21-1.56) | 0.91/0.91 | 0.94/0.93 |
| | | | | | Replication (JP2) | 1.20 (1.05-1.37) | 0.74 | 0.77 |
| | | | | | Combined | 1.29 (1.17-1.41) | | |
| 10q24.32 | rs12413409 | 104709086 | CNNM2 | G | Discovery | 1.38 (1.22-1.57) | 0.91/0.91 | 0.94/0.93 |
| | | | | | Replication | 1.23 (1.10-1.37) | 0.74 | 0.77 |
| | | | | | Combined | 1.29 (1.19-1.40) | | |
| 13q13.1 | rs9315204 | 32591837 | STARD13 | T | Discovery | 1.21 (1.13-1.31) | 0.21/0.33 | 0.24/0.39 |
| | | | | | Replication | 1.18 (1.06-1.31) | 0.24 | 0.27 |
| | | | | | Combined | 1.20 (1.13-1.28) | | |
| 13q13.1 | rs1960781 | 32598374 | STARD13 | G | Discovery | 1.21 (1.13-1.31) | 0.21/0.33 | 0.24/0.39 |
| | | | | | Replication | 1.18 (1.07-1.32) | 0.24 | 0.27 |
| | | | | | Combined | 1.20 (1.13-1.28) | | |
| 13q13.1 | rs3742321 | 32602065 | STARD13 | C | Discovery | 1.22 (1.13-1.31) | 0.21/0.33 | 0.24/0.39 |
| | | | | | Replication | 1.16 (1.05-1.29) | 0.24 | 0.27 |
| | | | | | Combined | 1.20 (1.13-1.27) | | |
| 18q11.2 | rs4800418 | 18400738 | RBBP8 | C | Discovery | 1.23 (1.15-1.32) | 0.30/0.31 | 0.35/0.34 |
| | | | | | Replication | 1.21 (1.09-1.36) | 0.21 | 0.24 |
| | | | | | Combined | 1.22 (1.15-1.30) | | |
| 18q11.2 | rs11662668 | 18433379 | RBBP8 | G | Discovery | 1.20 (1.13-1.29) | 0.48/0.44 | 0.54/0.47 |
| | | | | | Replication | 1.23 (1.12-1.35) | 0.60 | 0.64 |
| | | | | | Combined | 1.21 (1.15-1.28) | | |
| 18q11.2 | rs11661542 | 18477693 | RBBP8 | C | Discovery | 1.21 (1.14-1.30) | 0.49/0.44 | 0.54/0.47 |
| | | | | | Replication | 1.22 (1.11-1.34) | 0.61 | 0.65 |
| | | | | | Combined | 1.22 (1.15-1.28) | | |

Gene column indicates a region identifier, which does not imply that the listed gene is the causal one. In the Cohort column, “Replication (JP2)” means that, for the SNP rs12411886, JP1 cohort failed to genotype so that replication cohort includes only the JP2 cohort. Risk allele frequencies (RAFs) for discovery cohort are shown as (RAF of CE)/(RAF of FI).

TABLE 15
Sub-cohort results for 13 SNPs genotyped both in the discovery and
replication cohorts

| | | | | Risk | | | | | |
|---|---|---|---|---|---|---|---|---|---|
| Locus | SNP | Position | Gene | Allele | Cohort | P-value | log₁₀ (BF) | PPA | OR (95% CI) |
| 8q11.23 | rs1504749 | 55473264 | 5′-SOX17 | C | FI | 0.0028 | 1.23 | | 1.22 (1.07-1.40) |
| | | | | | NL | 0.00083 | 1.67 | | 1.28 (1.11-1.47) |
| | | | | | DE | 0.21 | −0.16 | | 1.10 (0.95-1.28) |
| | | | | | AN | 0.010 | 0.79 | | 1.29 (1.06-1.58) |
| | | | | | CE | 4.5E−05 | 2.77 | 0.0551 | 1.21 (1.10-1.33) |
| | | | | | JP1 | 0.033 | 0.41 | | 1.19 (1.01-1.39) |
| | | | | | JP2 | 0.65 | −0.49 | | 1.03 (0.91-1.17) |
| | | | | | FI + NL | 8.7E−06 | 3.43 | 0.2123 | 1.25 (1.13-1.37) |
| | | | | | DE + AN | 0.011 | 0.75 | 0.0006 | 1.17 (1.04-1.32) |
| 8q11.23 | rs10958409 | 55489644 | 5′-SOX17 | A | FI | 0.0051 | 1.03 | | 1.23 (1.06-1.41) |
| | | | | | NL | 0.00011 | 2.34 | | 1.36 (1.16-1.59) |
| | | | | | DE | 0.30 | −0.22 | | 1.09 (0.93-1.29) |
| | | | | | AN | 0.013 | 0.71 | | 1.32 (1.06-1.65) |
| | | | | | CE | 2.3E−05 | 3.02 | 0.0955 | 1.25 (1.12-1.38) |
| | | | | | JP1 | 0.061 | 0.23 | | 1.17 (0.99-1.38) |
| | | | | | JP2 | 0.60 | −0.46 | | 1.04 (0.91-1.18) |
| | | | | | FI + NL | 3.3E−06 | 3.81 | 0.3902 | 1.28 (1.16-1.43) |
| | | | | | DE + AN | 0.021 | 0.54 | 0.0003 | 1.17 (1.02-1.33) |
| 8q12.1 | rs9298506 | 55600077 | 3′-SOX17 | A | FI | 1.0E−05 | 3.22 | | 1.39 (1.20-1.61) |
| | | | | | NL | 4.0E−05 | 2.66 | | 1.43 (1.20-1.69) |
| | | | | | DE | 0.11 | 0.05 | | 1.15 (0.97-1.36) |
| | | | | | AN | 0.011 | 0.76 | | 1.34 (1.07-1.69) |
| | | | | | CE | 1.6E−06 | 4.04 | 0.5233 | 1.30 (1.17-1.45) |
| | | | | | JP1 | 0.19 | −0.06 | | 1.14 (0.94-1.38) |
| | | | | | JP2 | 0.0022 | 1.33 | | 1.25 (1.08-1.44) |
| | | | | | FI + NL | 2.2E−09 | 6.65 | 0.9978 | 1.41 (1.26-1.57) |
| | | | | | DE + AN | 0.0053 | 1.02 | 0.0011 | 1.22 (1.06-1.39) |
| 9p21.3 | rs1333040 | 22073404 | CDKN2A/B | T | FI | 5.3E−08 | 5.29 | | 1.39 (1.23-1.56) |
| | | | | | NL | 4.8E−05 | 2.72 | | 1.30 (1.15-1.48) |
| | | | | | DE | 5.4E−05 | 2.66 | | 1.31 (1.15-1.49) |
| | | | | | AN | 0.0080 | 0.88 | | 1.25 (1.06-1.48) |
| | | | | | CE | 3.8E−10 | 7.41 | 0.9996 | 1.29 (1.19-1.40) |
| | | | | | JP1 | 3.4E−05 | 2.76 | | 1.41 (1.20-1.66) |
| | | | | | JP2 | 0.00040 | 1.94 | | 1.26 (1.11-1.43) |
| | | | | | FI + NL | 1.9E−11 | 8.69 | 1.0-2.1E−05 | 1.35 (1.24-1.47) |
| | | | | | DE + AN | 1.6E−06 | 4.08 | 0.5465 | 1.29 (1.16-1.43) |
| 9p21.3 | rs2383207 | 22105959 | CDKN2A/B | G | FI | 4.7E−07 | 4.48 | | 1.35 (1.20-1.52) |
| | | | | | NL | 4.0E−05 | 2.79 | | 1.30 (1.14-1.47) |
| | | | | | DE | 1.4E−05 | 3.17 | | 1.32 (1.17-1.50) |
| | | | | | AN | 0.11 | 0.06 | | 1.14 (0.97-1.34) |
| | | | | | CE | 2.1E−09 | 6.73 | 0.9981 | 1.27 (1.17-1.37) |
| | | | | | JP1 | 0.00085 | 1.65 | | 1.30 (1.12-1.52) |
| | | | | | JP2 | 7.2E−06 | 3.43 | | 1.33 (1.17-1.50) |
| | | | | | FI + NL | 1.1E−10 | 7.97 | 0.9999 | 1.33 (1.22-1.44) |
| | | | | | DE + AN | 1.1E−05 | 3.34 | 0.1783 | 1.25 (1.13-1.38) |
| 10q24.32 | rs12411886 | 104675289 | CNNM2 | C | FI | 0.043 | 0.36 | | 1.28 (1.01-1.59) |
| | | | | | NL | 0.12 | 0.10 | | 1.22 (0.95-1.56) |
| | | | | | DE | 4.4E−05 | 2.29 | | 1.67 (1.30-2.14) |
| | | | | | AN | 0.022 | 0.49 | | 1.44 (1.05-1.97) |
| | | | | | CE | 5.1E−06 | 3.45 | 0.2204 | 1.43 (1.23-1.67) |
| | | | | | JP1 | NA | NA | | NA (NA-NA) |
| | | | | | JP2 | 0.0092 | 0.82 | | 1.20 (1.05-1.37) |
| | | | | | FI + NL | 0.011 | 0.77 | 0.0006 | 1.24 (1.05-1.47) |
| | | | | | DE + AN | 4.6E−06 | 3.34 | 0.1799 | 1.58 (1.30-1.92) |
| 10q24.32 | rs12413409 | 104709086 | CNNM2 | G | FI | 0.042 | 0.37 | | 1.27 (1.01-1.59) |
| | | | | | NL | 0.096 | 0.15 | | 1.23 (0.96-1.58) |
| | | | | | DE | 4.9E−05 | 2.27 | | 1.66 (1.30-2.13) |
| | | | | | AN | 0.021 | 0.50 | | 1.44 (1.05-1.97) |
| | | | | | CE | 3.9E−06 | 3.55 | 0.2613 | 1.44 (1.23-1.68) |
| | | | | | JP1 | 0.0073 | 0.91 | | 1.27 (1.07-1.50) |
| | | | | | JP2 | 0.0062 | 0.96 | | 1.21 (1.05-1.38) |
| | | | | | FI + NL | 0.0089 | 0.85 | 0.0007 | 1.25 (1.06-1.48) |
| | | | | | DE + AN | 4.9E−06 | 3.32 | 0.1728 | 1.58 (1.30-1.91) |
| 13q13.1 | rs9315204 | 32591837 | STARD13 | T | FI | 0.00017 | 2.25 | | 1.27 (1.12-1.44) |
| | | | | | NL | 0.0026 | 1.26 | | 1.25 (1.08-1.45) |
| | | | | | DE | 0.11 | 0.04 | | 1.13 (0.97-1.32) |
| | | | | | AN | 0.14 | 0.02 | | 1.15 (0.95-1.40) |
| | | | | | CE | 0.00034 | 1.99 | 0.0096 | 1.19 (1.08-1.30) |
| | | | | | JP1 | 0.078 | 0.15 | | 1.16 (0.98-1.37) |
| | | | | | JP2 | 0.010 | 0.79 | | 1.20 (1.04-1.37) |
| | | | | | FI + NL | 1.6E−06 | 4.09 | 0.5532 | 1.26 (1.15-1.39) |
| | | | | | DE + AN | 0.029 | 0.40 | 0.0003 | 1.14 (1.01-1.29) |
| 13q13.1 | rs1980781 | 32598374 | STARD13 | G | FI | 0.00024 | 2.13 | | 1.26 (1.11-1.43) |
| | | | | | NL | 0.0024 | 1.29 | | 1.25 (1.08-1.45) |
| | | | | | DE | 0.090 | 0.09 | | 1.14 (0.98-1.33) |
| | | | | | AN | 0.16 | −0.03 | | 1.15 (0.95-1.39) |
| | | | | | CE | 0.00031 | 2.03 | 0.0105 | 1.19 (1.08-1.30) |
| | | | | | JP1 | 0.068 | 0.20 | | 1.17 (0.99-1.39) |
| | | | | | JP2 | 0.011 | 0.76 | | 1.19 (1.04-1.37) |
| | | | | | FI + NL | 2.0E−06 | 4.01 | 0.5035 | 1.26 (1.14-1.38) |
| | | | | | DE + AN | 0.028 | 0.41 | 0.0003 | 1.14 (1.01-1.29) |
| 13q13.1 | rs3742321 | 32602065 | STARD13 | C | FI | 0.00018 | 2.23 | | 1.27 (1.12-1.43) |
| | | | | | NL | 0.0018 | 1.39 | | 1.26 (1.09-1.46) |
| | | | | | DE | 0.096 | 0.08 | | 1.14 (0.98-1.33) |
| | | | | | AN | 0.18 | −0.05 | | 1.14 (0.94-1.38) |
| | | | | | CE | 0.00030 | 2.04 | 0.0109 | 1.19 (1.08-1.30) |
| | | | | | JP1 | 0.14 | −0.01 | | 1.13 (0.96-1.34) |
| | | | | | JP2 | 0.015 | 0.65 | | 1.18 (1.03-1.35) |
| | | | | | FI + NL | 1.2E−06 | 4.21 | 0.6197 | 1.26 (1.15-1.39) |
| | | | | | DE + AN | 0.033 | 0.36 | 0.0002 | 1.14 (1.01-1.29) |
| 18q11.2 | rs4800418 | 18400738 | RBBP8 | C | FI | 0.037 | 0.34 | | 1.14 (1.01-1.30) |
| | | | | | NL | 5.0E−05 | 2.69 | | 1.31 (1.15-1.50) |
| | | | | | DE | 0.00015 | 2.28 | | 1.30 (1.13-1.49) |
| | | | | | AN | 0.14 | 0.0017 | | 1.14 (0.96-1.36) |
| | | | | | CE | 2.7E−08 | 5.70 | 0.9804 | 1.27 (1.17-1.38) |
| | | | | | JP1 | 0.00019 | 2.11 | | 1.40 (1.17-1.68) |
| | | | | | JP2 | 0.13 | −0.05 | | 1.11 (0.97-1.28) |
| | | | | | FI + NL | 1.7E−05 | 3.16 | 0.1266 | 1.22 (1.12-1.34) |
| | | | | | DE + AN | 1.0E−04 | 2.48 | 0.0294 | 1.24 (1.11-1.37) |
| 18q11.2 | rs11662668 | 18433379 | RBBP8 | G | FI | 0.037 | 0.31 | | 1.13 (1.01-1.27) |
| | | | | | NL | 0.0029 | 1.22 | | 1.21 (1.07-1.37) |
| | | | | | DE | 2.0E−05 | 3.04 | | 1.32 (1.16-1.50) |
| | | | | | AN | 0.061 | 0.23 | | 1.17 (0.99-1.39) |
| | | | | | CE | 8.6E−08 | 5.24 | 0.9460 | 1.24 (1.15-1.34) |
| | | | | | JP1 | 0.011 | 0.79 | | 1.21 (1.05-1.41) |
| | | | | | JP2 | 0.00037 | 1.97 | | 1.24 (1.10-1.40) |
| | | | | | FI + NL | 0.00039 | 1.93 | 0.0084 | 1.17 (1.07-1.27) |
| | | | | | DE + AN | 6.4E−06 | 3.55 | 0.2602 | 1.26 (1.14-1.40) |
| 18q11.2 | rs11661542 | 18477693 | RBBP8 | C | FI | 0.023 | 0.47 | | 1.14 (1.02-1.28) |
| | | | | | NL | 0.00087 | 1.66 | | 1.23 (1.09-1.40) |
| | | | | | DE | 4.4E−05 | 2.74 | | 1.30 (1.15-1.48) |
| | | | | | AN | 0.044 | 0.33 | | 1.19 (1.00-1.40) |
| | | | | | CE | 3.3E−08 | 5.63 | 0.9772 | 1.25 (1.15-1.35) |
| | | | | | JP1 | 0.0061 | 0.97 | | 1.24 (1.06-1.46) |
| | | | | | JP2 | 0.0023 | 1.31 | | 1.20 (1.07-1.36) |
| | | | | | FI + NL | 8.9E−05 | 2.51 | 0.0312 | 1.18 (1.09-1.29) |
| | | | | | DE + AN | 8.6E−06 | 3.43 | 0.2121 | 1.26 (1.14-1.39) |

Sub-cohorts of CE (NL, DE and AN) were defined based on the case series (Table 13):
NL = NL cases (n = 708) and matched controls (n = 3,954);
DE = DE cases (n = 789) and matched controls (n = 2,228); and
AN = @neurIST cases (n = 475) and matched controls (n = 1,940).
FI + NL data includes only previously analyzed cases (and their matched controls), while DE + AN data includes newly analyzed cases (and their matched controls).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying an single nucleotide polymorphism (SNP) associated with intracranial aneurysm in a biological sample of a subject in need thereof, the method comprising:
a. obtaining a biological sample from the subject,
b. determining the sequence of at least a portion of the subject's nucleic acid,
c. comparing the subject's nucleic acid sequence to a wild-type nucleic acid sequence, and
d. identifying at least one SNP in the subject's nucleic acid sequence as compared with the wild-type nucleic acid sequence,
wherein the identified SNP is associated with intracranial aneurysm.

2. The method of claim 1, wherein the nucleic acid is at least one selected from the group consisting of: genomic DNA, mRNA and cDNA.

3. The method of claim 1, wherein the at least one SNP is at least one selected from the group consisting of: rs6841581, rs9298506, rs1333040, rs12413409, rs6538595, rs9315204, rs11661542, and rs1132274.

4. The method of claim 1, wherein the subject is human.

5. The method of claim 1, wherein said step of determining the sequence of the subject's nucleic employs PCR.

6. The method of claim 1, wherein the biological sample is at least one selected from the group consisting of: blood, plasma, serum, a body fluid, a cell, and a tissue.

7. A method of diagnosing a subject as having, or as being at risk of developing, intracranial aneurysm, the method comprising:
a. obtaining a biological sample from the subject,
b. determining the sequence of at least a portion of the subject's nucleic acid,
c. comparing the subject's nucleic acid sequence to a wild-type nucleic acid sequence, and
d. identifying at least one SNP in the subject's nucleic acid sequence as compared with the wild-type nucleic acid sequence,
wherein the identified SNP is associated with intracranial aneurysm.

8. The method of claim 7, wherein the nucleic acid is at least one selected from the group consisting of: genomic DNA, mRNA and cDNA.

9. The method of claim 7, wherein the at least one SNP is at least one selected from the group consisting of: rs6841581, rs9298506, rs1333040, rs12413409, rs6538595, rs9315204, rs11661542, and rs1132274.

10. The method of claim 7, wherein the subject is human.

11. The method of claim 7, wherein said step of determining the sequence of the subject's nucleic employs PCR.

12. The method of claim 7, wherein the biological sample is at least one selected from the group consisting of: blood, plasma, serum, a body fluid, a cell, and a tissue.

13. A method of treating intracranial aneurysm in a subject in need thereof, the method comprising: administering to the subject, a therapeutically effective amount of a modulator of a gene, or gene product, that is associated with an SNP that is associated with intracranial aneurysm, wherein the subject has been diagnosed as having an intracranial aneurysm, and wherein after the modulator is administered to the subject, the intracranial aneurysm is treated.

14. The method of claim 13, wherein the modulator is at least one selected from the group consisting of: a chemical compound, a polypeptide, a peptide, a peptidomemetic, an antibody, a ribozyme, a small molecule chemical compound, and an antisense nucleic acid molecule.

15. The method of claim 13, wherein the subject has at least one SNP associated with intracranial aneurysm.

16. The method of claim 15, wherein the SNP associated with intracranial aneurysm is at least one selected from the group consisting of: rs6841581, rs9298506, rs1333040, rs12413409, rs6538595, rs9315204, rs11661542, and rs1132274.

17. The method of claim 13, wherein the gene, or gene product, that is associated with an SNP associated with intracranial aneurysm is at least one selected from the group consisting of: EDNRA, SOX17, CDKN2A, CDKN2B, CNNM2, NDUFA12, NR2C1, FGD6, VEZT, KL, STARD13, RBBP8, DSTN, and RRBP1.

18. The method of claim 13, wherein the subject is human.

19. A method of preventing intracranial aneurysm in a subject at risk thereof, the method comprising: administering to the subject, a therapeutically effective amount of a modulator of a gene, or gene product, that is associated with an SNP that is associated with intracranial aneurysm, wherein the subject has been diagnosed as being at risk of developing an intracranial aneurysm, and wherein after the modulator is administered to the subject, the intracranial aneurysm is prevented.

20. The method of claim 19, wherein the modulator is at least one selected from the group consisting of: a chemical compound, a polypeptide, a peptide, a peptidomemetic, an antibody, a ribozyme, a small molecule chemical compound, and an antisense nucleic acid molecule.

21. The method of claim 19, wherein the subject has at least one SNP associated with intracranial aneurysm.

22. The method of claim 21, wherein the SNP associated with intracranial aneurysm is at least one selected from the group consisting of: rs6841581, rs9298506, rs1333040, rs12413409, rs6538595, rs9315204, rs11661542, and rs1132274.

23. The method of claim 19, wherein the gene, or gene product, that is associated with an SNP associated with intracranial aneurysm is at least one selected from the group consisting of: EDNRA, SOX17, CDKN2A, CDKN2B, CNNM2, NDUFA12, NR2C1, FGD6, VEZT, KL, STARD13, RBBP8, DSTN, and RRBP1.

24. The method of claim 19, wherein the subject is human.

25. A method of identifying a test compound as a modulator of a gene, or gene product, that is associated with an SNP associated with intracranial aneurysm, the method comprising:
a. measuring at least one parameter of a gene, or gene product, that is associated with an SNP associated with intracranial aneurysm in the absence of the test compound,
b. measuring the at least one parameter of a gene, or gene product, that is associated with an SNP associated with intracranial aneurysm in the presence of the test compound;
c. comparing the measurement of the at least one parameter of a gene, or gene product, that is associated with an SNP associated with intracranial aneurysm in the presence of the test compound with the measurement in the absence of the test compound;
d. identifying the test compound as a modulator of the gene, or gene product, that is associated with an SNP associated with intracranial aneurysm when the measurement of the parameter in the presence of the test compound is different than the measurement of the parameter in the absence of the test compound.

26. The method of claim 25, wherein when the measurement of the parameter is higher in the presence of the test compound, the test compound is identified as an activator.

27. The method of claim 25, wherein when the measurement of the parameter is lower in the presence of the test compound, the test compound is identified as an inhibitor.

28. The method of claim 25, wherein the test compound is at least one selected from the group consisting of: a chemical compound, a polypeptide, a peptide, a peptidomemetic, an antibody, a nucleic acid, an antisense nucleic acid, an shRNA, a ribozyme, and a small molecule chemical compound.

29. A modulator identified using the method of claim 25.

30. The modulator of claim 29, wherein the modulator is an activator.

31. The modulator of claim 29, wherein the modulator is an inhibitor.

32. A composition comprising the modulator identified using the method of claim 25.
